(19) United States
(12) Patent Application Publication
(10) Pub. No.: US 2022/0133136 A1
(43) Pub. Date: May 5, 2022

(54) ACCESS DEVICE

(71) Applicant: Grumpy Innovation, Inc., New York City, NY (US)

(72) Inventors: Sameer SHARMA, New York, NY (US); Adam J.M. Larson, Reno, NV (US); Matthew Bymes Newell, Reno, NV (US); Luke W. Clauson, Reno, NV (US)

(73) Assignee: Grumpy Innovation, Inc., New York City, NY (US)

(21) Appl. No.: 17/647,832

(22) Filed: Jan. 12, 2022

Related U.S. Application Data
(63) Continuation of application No. PCT/US2020/041813, filed on Jul. 13, 2020.
(60) Provisional application No. 62/873,457, filed on Jul. 12, 2019.

(51) Int. Cl. A61B 1/00

(52) U.S. Cl. CPC A61B 1/00148; A61B 1/00135

(57) ABSTRACT

Tissue access devices and methods of using the same are disclosed. For example, an access device is disclosed having a first sheath having a first sheath lumen, a second sheath having a second sheath lumen, and an engager. The second sheath can be deflectable into and out of the first sheath lumen. The engager can be expandable and contractible. When the engager is in an expanded configuration, a space can be between the engager and the first sheath and the second sheath can be deflectable into and out of the space.

### CROSS-REFERENCE TO RELATED APPLICATION

This application is a continuation of International Patent Application No. PCT/US2020/041813 filed Jul. 13, 2020, which claims the benefit of priority to U.S. Provisional Application No. 62/873,457 filed Jul. 12, 2019, each of which is incorporated herein by reference in its entirety for all purposes.

### BACKGROUND

### 1. Technical Field

This disclosure relates generally access devices and methods of using the same, and more particularly to endoscopic retrograde cholangiopancreatography (ERCP).

### 2. Background of the Art

Pancreaticobiliary endoscopy focuses on the diagnosis and therapy of conditions involving the pancreas and biliary tree, and distinguishes itself from standard lumenal endoscopy by a greater use of side-viewing endoscopes and echoendoscopes and the use of fluoroscopy.

However, ERCP remains a firmly established therapeutic modality that offers a wide range of interventions for pancreaticobiliary disease. Skillful execution of diagnostic ERCP remains crucial for the safe and effective deployment of endoscopic therapies. Endoscopic retrograde cholangiopancreatography involves the endoscopic cannulation of the major papilla with imaging of the biliary tree and the pancreatic ductal system.

ERCP is typically performed using side-viewing endoscopes and guidewires. Cannulation may initially be attempted either by gently threading the guidewire 1 to 2 cm into the desired channel (wire-guided cannulation) or by gentle impaction of the catheter tip in the papillary orifice. However, wire-guided cannulation must be performed carefully, as unintended passage of the guidewire into the pancreatic duct is common during biliary cannulation, and is associated with pancreatitis. Wire-induced pancreatic duct perforation through a side branch (often at the genu) can occur if the guidewire is advanced beyond 2 cm without contrast agent injection to confirm position. Soft-tipped wires have the advantage of inducing less tissue trauma (e.g., fewer submucosal or other extraductal dissections). Torsion with advancement of an angled wire with a hydrophilic tip may facilitate access across angulated ductal anatomy. Ultimately, however, the specific devices used are much less important than the experience and skill of the endoscopist and the ERCP team members. The experience and skill of the endoscopist and the ERCP team members become even more important for when performing ERCP in patients with altered surgical anatomy.

During the course of an ERCP procedure, it is common for undesirable endoscope movements because of tension in the endoscope shaft and because of patient movement and small intestine peristalsis. This makes it difficult for a doctor to cannulate the Ampulla of Vater, and thereon the common bile duct and pancreatic duct selectively, a key step in the ERCP procedure, and which must be accomplished before the ERCP procedure can be completed.

Accordingly, a need exists for an improved access device that can increase success rates of ERCP procedures, reduce procedure times of ERCP procedures, provide stability for the endoscope and endoscopic tools during the ERCP procedure, improve visualization of the Ampulla of Vater, and align a working channel with the Ampulla of Vater for simple cannulation thereof using an end-viewing endoscope.

### SUMMARY

Access devices are disclosed. Methods of accessing targets are disclosed. Methods of using access devices are disclosed. Methods of making access devices are disclosed.

Access devices are disclosed. For example, an access device having a first sheath is disclosed. The first sheath can have a first sheath lumen. The access device can have a second sheath having a second sheath lumen. The second sheath can be deflectable into and out of the first sheath lumen. The second sheath can have a deflected configuration and a non-deflected configuration. When the second sheath is in the deflected configuration, a second sheath first portion can be in the first sheath lumen and can extend across the first sheath lumen. When the second sheath is in the non-deflected configuration, the second sheath first portion can be out of the first sheath lumen. The access device can have an engager. The engager can be expandable and contractible. The engager can have an expanded configuration and a contracted configuration. When the engager is in the expanded configuration, a space can be between the engager and the first sheath. When the engager is in the expanded configuration, a second sheath second portion can be deflectable into the space. When the engager is in the expanded configuration and when the second sheath is in the deflected configuration, the second sheath second portion can be in the space. When the engager is in the expanded configuration and when the second sheath is in the non-deflected configuration, the second sheath second portion can be out of the space.

The second sheath second portion can be a distal terminal end of the second sheath.

When the second sheath is in the non-deflected configuration, the second sheath first portion can be straight. When the second sheath is in the deflected configuration, the second sheath first portion can be curved.

When the second sheath is in the deflected configuration, the second sheath first portion can be more curved than when the second sheath is in the non-deflected configuration.

When the second sheath is in the deflected configuration, the second sheath first and second portions can define a hook shape.

When the second sheath is in the non-deflected configuration, the second sheath first portion can be parallel to the first sheath lumen.

The first sheath can have a first sheath second lumen. The second sheath can be in the first sheath second lumen.

The engager can have a stabilizer. The stabilizer can have an expanded configuration and a contracted configuration. The stabilizer can be moveable from the contracted configuration to the expanded configuration. When the stabilizer is in the expanded configuration, the stabilizer can be farther from the first sheath than when the stabilizer is in the contracted configuration.

The engager can have an expander and a stabilizer. The expander can be expandable and contractible. The expander can have an expanded configuration and a contracted configuration. When the expander is in the expanded configuration, the engager can be in the expanded configuration. When the expander is in the contracted configuration, the engager can be in the contracted configuration. The stabilizer can have an expanded configuration and a contracted configuration. The stabilizer can be moveable from the contracted configuration to the expanded configuration via the expander. When the stabilizer is in the expanded configuration, the stabilizer can be farther from the first sheath than when the stabilizer is in the contracted configuration.

The expander can include a balloon.

The access device can have an endoscope, a third sheath, and a guidewire. The endoscope can be moveable in the first sheath lumen. The third sheath and the guidewire can be moveable in the second sheath lumen when the second sheath is in the deflected configuration.

The access device can have a third sheath having a third sheath first lumen and a third sheath second lumen. The first sheath can be in the third sheath first lumen. The second sheath can be in the third sheath second lumen.

The first sheath can be a torque carrier.

The access device can have a torque carrier attached to the first sheath.

Access devices are disclosed. For example, an access device is disclosed having a first sheath. The first sheath can have a first sheath lumen and a first sheath distal tip. The first sheath distal tip can be moveable away from and toward the first sheath lumen. The first sheath can have a first sheath deflected configuration and a first sheath non-deflected configuration. When the first sheath is in the first sheath deflected configuration, the first sheath distal tip can be farther from the first sheath lumen than when the first sheath is in the first sheath non-deflected configuration. The access device can have a second sheath having a second sheath lumen. The second sheath can be deflectable into and out of the first sheath lumen. The second sheath can have a second sheath deflected configuration and a second sheath non-deflected configuration. When the second sheath is in the second sheath deflected configuration, a second sheath first portion can be inside the first sheath lumen and can extend across the first sheath lumen. When the second sheath is in the second sheath non-deflected configuration, the second sheath first portion can be outside the first sheath lumen. The access device can have a stabilizer. The stabilizer can be expandable and contractible. The stabilizer can have an expanded configuration and a contracted configuration. The stabilizer can be moveable from the contracted configuration to the expanded configuration via the first sheath distal tip. When the first sheath is in the first sheath non-deflected configuration, the stabilizer can be in the contracted configuration. When the first sheath is in the first sheath deflected configuration, the stabilizer can be in the expanded configuration. When the stabilizer is in the expanded configuration, the stabilizer can be farther from a first sheath lumen longitudinal axis than when the stabilizer is in the contracted configuration.

The stabilizer can have a stabilizer first end and a stabilizer second end. When the stabilizer is in the expanded configuration, the stabilizer first end can be closer to the first sheath lumen longitudinal axis than the stabilizer second end.

The access device can have an endoscope, a third sheath, and a guidewire. The endoscope can be moveable in the first sheath lumen. The third sheath and the guidewire can be moveable in second sheath lumen when the second sheath is in the deflected configuration.

When the stabilizer is in the expanded configuration, a space can be between the stabilizer and the first sheath. When the stabilizer is in the expanded configuration, a second sheath second portion can be deflectable into the space. When the stabilizer is in the expanded configuration and when the second sheath is in the deflected configuration, the second sheath second portion can be in the space. When the stabilizer is in the expanded configuration and when the second sheath is in the non-deflected configuration, the second sheath second portion can be out of the space.

A method of accessing a target in a body lumen is disclosed. The method can include advancing a first sheath, a second sheath, and an engager to the target. The first sheath can have a first sheath lumen and the second sheath can have a second sheath lumen. The method can include creating a space between the target and the engager by expanding the engager. The method can include deflecting a distal tip of the second sheath transversely across the first sheath lumen and into the space. The method can include advancing a third sheath through the second sheath lumen and into the target. The third sheath can have a third sheath lumen. The method can include advancing a tool through the third sheath lumen into the target.

The tool can be a guidewire.

### BRIEF SUMMARY OF THE DRAWINGS

The drawings shown and described are exemplary embodiments and non-limiting. Like reference numerals indicate identical or functionally equivalent features throughout.

FIG. 1 illustrates a variation of a cross-sectional view of an access device in an expanded state through lines F1-F1 in FIG. 2.

FIG. 2 illustrates a variation of an isometric view of the access device of FIG. 1.

FIG. 3 illustrates a variation of an endoscope view.

FIG. 4 illustrates a variation of a cross-sectional view of an access device in a partially expanded state.

FIG. 5 illustrates a variation of an isometric view of the access device of FIG. 4.

FIG. 6 illustrates a variation of a cross-sectional view of an access device in an unexpanded state.

FIG. 7 illustrates a variation of an isometric view of the access device of FIG. 6.

FIG. 8 illustrates a variation of a split shaft in an open configuration.

FIG. 9 illustrates a variation of a split shaft of FIG. 8 in a closed configuration.

FIGS. 10A-10C illustrate a variation of a variety of expansion states.

FIG. 11 illustrates a variation of transverse cross-sectional view of an access device having a variation of a tissue manipulator.

FIG. 12 illustrates a variation of a longitudinal cross-sectional view of the access device of FIG. 11.

FIG. 13 illustrates a variation of a transverse cross-sectional view of an access device having a variation of a tissue manipulator.

FIG. 14 illustrates a variation of a transverse cross-sectional view of an access device having a working channel with a beveled tip geometry.

FIG. 15 illustrates a variation of a longitudinal cross-sectional view of the access device of FIG. 15.

FIG. 16 illustrates a variation of a working channel having a variation of a one-way check valve.

FIG. 17 illustrates a variation of an endoscope view an access device having a variation of skirt balloons.

FIG. 18 illustrates a variation of a longitudinal cross-sectional view of the device of FIG. 17.

FIG. 19 illustrates a variation of a transverse cross-sectional view of an access device.

FIG. 20 illustrates a variation of an isometric view of the access device of FIG. 19.

FIG. 21 illustrates a variation of an isometric view of an access device.

FIG. 22 illustrates a variation of an isometric view of an access device.

FIG. 23 illustrates a variation of an isometric view of an access device.

FIG. 24 illustrates a variation of a magnified view of the access device of FIG. 23.

FIG. 25 illustrates a variation of a transverse cross-sectional view of the access device of FIG. 23.

FIG. 26 illustrates a variation of a stabilizer holder.

FIG. 27 illustrates a variation of a transverse cross-sectional view of an access device.

FIG. 28 illustrates a variation of an access device having a variation of a bridge for tissue manipulation.

FIG. 29 illustrates a transverse cross-sectional view of the access device of FIG. 29.

FIG. 30A illustrates a side view of a variation of an access device.

FIG. 30B illustrates a close-up view of the access device of FIG. 30A at section 30B.

FIG. 30C illustrates a top view of the access device of FIG. 30A.

FIG. 30D illustrates a close-up view of the access device of FIG. 30C at section 30D.

FIG. 30E illustrates a close-up view of the access device of FIG. 30C at section 30E.

FIG. 30F illustrates a side view of the access device of FIG. 30A with a variation of a torque carrier.

FIG. 30G illustrates a close-up view of the access device of FIG. 30F at section 30G.

FIG. 30H illustrates a close-up view of the access device of FIG. 30F at section 30H.

FIG. 30I illustrates a top view of the access device of FIG. 30F at section 30I.

FIG. 30J illustrates a perspective view of the access device of FIG. 30I at section 30J.

FIG. 30K illustrates a top view of the access device of FIG. 30F in a curved configuration.

FIG. 31A illustrates a side view of the access device of FIG. 30A with a variation of a torque carrier.

FIG. 31B illustrates a perspective view of the access device of FIG. 31A at section 30B.

FIG. 31C illustrates a variation of a cross-section view of the access device of FIG. 31A through the lines 31C-31C.

FIG. 32A illustrates a perspective view of a variation of a sheath for an access device.

FIG. 32B illustrates a perspective view of a variation of a torque carrier for an access device.

FIG. 32C illustrates a variation of a cross-section view of the access device of FIG. 32A through the lines 32C-32C.

FIG. 32D illustrates a side view of a variation of an access device with the torque carrier of FIG. 32B.

FIG. 32E illustrates a perspective view of the access device of FIG. 32D at section 32E.

FIG. 33A illustrates a side view of a variation of an expander.

FIG. 33B illustrates a perspective view of a variation of an expander.

FIG. 34A illustrates a side view of a variation of an access device.

FIG. 34B illustrates a perspective view of a variation of an access device.

### DETAILED DESCRIPTION

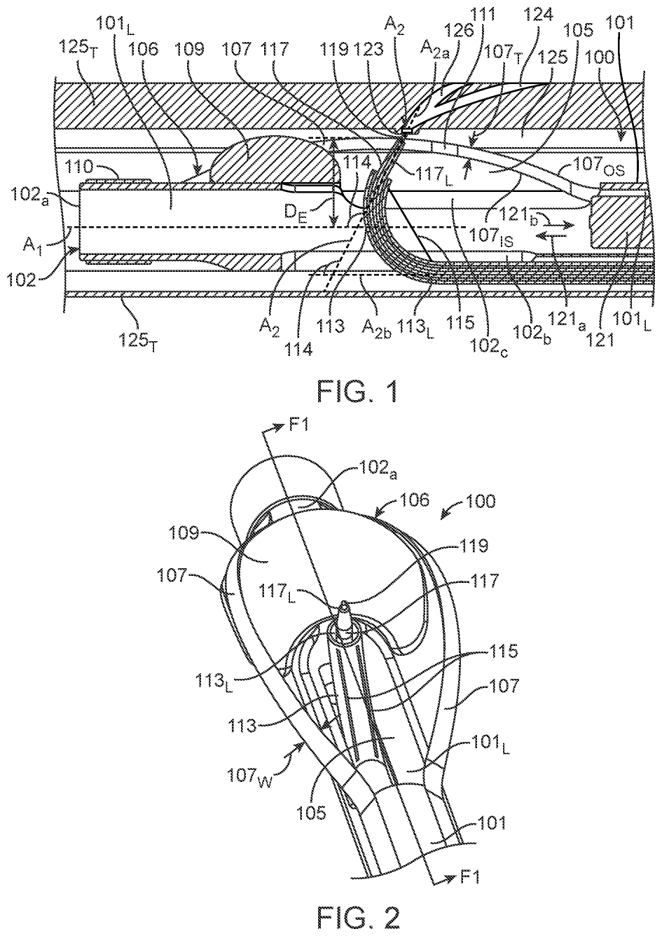

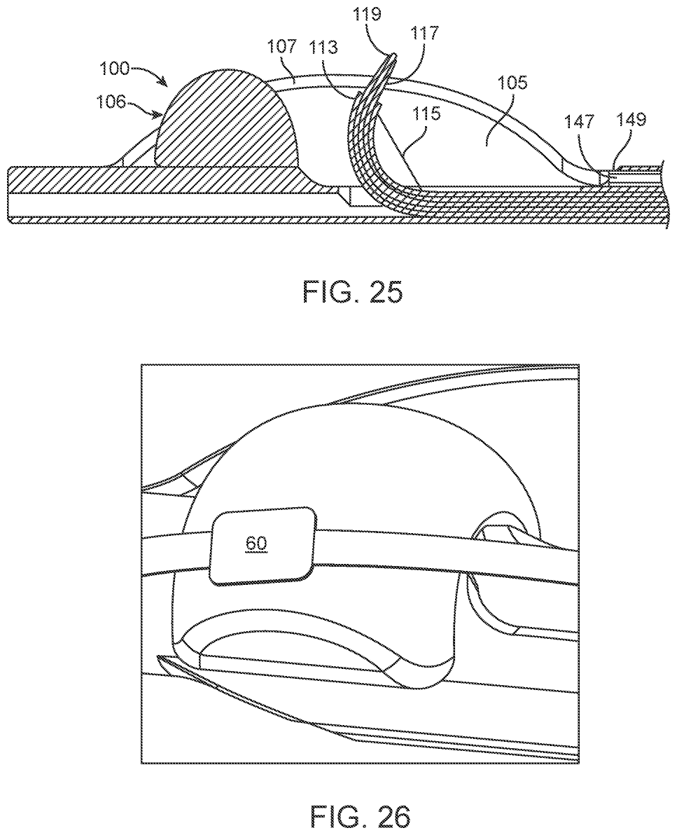

The features illustrated in FIGS. 1-34B can be combined with each other in any combination. The features described in this specification can be combined with each other in any combination.

FIG. 1 illustrates a variation of an access device 100 (also referred to as the device 100) that can be inserted into lumens, for example, into lumens of anatomical structures of a person (also referred to as body lumens). The lumens can be, for example, part of the person's gastrointestinal anatomy, respiratory anatomy, reproductive anatomy, vascular anatomy, or urinary anatomy. For example, FIG. 1 illustrates that the device can be inserted into an intestinal lumen 125.

The device 100 can be a cannulation device, a visualization device, a tissue engagement device, a deployment device (e.g., tool deployment device, implant deployment device), or any combination thereof. FIG. 1 illustrates, for example, that the device 100 can be a cannulation device, a visualization device, a tissue engagement device, and a deployment device. The device 100 can visualize a target, can cannulate the target, can engage with tissue, and tools (e.g., guidewires, papillotomes) can be deployed from the device 100.

The device 100 can cannulate a target (e.g., a space, lumen, channel, duct) from the body lumen that the device 100 is in (e.g., the intestinal lumen 125) to access other spaces, lumens, channels, or ducts in the body. For example, the device 100 can be a cannulator that can visualize tissue (e.g., with a camera, with an endoscope) to perform, for example, retrograde cholangiopancreatography (ERCP) procedures. The device 100 can be an ERCP assist device. The device 100 can be used to perform ERCP procedures. For example, FIG. 1 illustrates that the device 100 can cannulate the Ampulla of Vater 123 to access the bile duct 124 or the pancreatic duct 126 from the intestinal lumen 125.

The device 100 can advantageously (1) provide stability for a fixed or moveable camera, (2) provide stability for an endoscope and/or endoscopic tools (e.g., during ERCP procedures), (3) improve visualization of lumens (e.g., the intestinal lumen 125) and cannulation targets (e.g., the Ampulla of Vater), (4) have a moveable working channel to more accurately and/or reliably cannulate a target which can decrease the amount of time needed to cannulate the target and thereby reduce the overall length of procedures involving cannulation, (5) have accessory channels to facilitate cannulation, or any combination thereof.

For example, FIG. 1 illustrates that the device 100 can have a moveable working channel 113_{L} that can be moved to different positions to match or closely approximate the entrance angle desired or needed to cannulate a target (e.g., the Ampulla of Vater 123). The device 100 can have an expandable and contractible engager 106 (also referred to as a tissue engager 106) that can stabilize the device 100 in the lumen (e.g., in the intestinal lumen 125), for example, by engaging with the wall of the lumen when in an expanded configuration. When device 100 is secured in the lumen via the tissue engager 106, longitudinal and/or rotational movement of the device 100 in the lumen can be inhibited or prevented. The moveable working channel 113_{L} can be moved to different positions when the device 100 is in a secured or unsecured position in the lumen via the tissue engager 106. The device 100 can be in a secured position in the lumen when the tissue engager 106 is in an expanded configuration or is engaged with tissue. The device 100 can be in an unsecured position in the lumen when the tissue engager 106 is in an unexpanded configuration or is not engaged with tissue. The working channel 113_{L} can be moved to a desired position to cannulate the target when the device 100 is in a secured or unsecured position. However, first securing the device 100 in the lumen via the tissue engager 106 can advantageously stabilize the device 100 relative to the target so that the working channel 113_{L} can be more easily moved to a desired cannulation position or a tool deployment position. The device 100 can have a camera, an endoscope 121, or both. The camera and/or the endoscope 121 can be used to steer the device 100, to cannulate the target, or both.

These features and advantages of the device 100 can be used in various procedures. In one variation of a procedure, the device 100 can be guided to a target (e.g., the Ampulla of Vater 123) by steering the device 100 through lumens in the body by using images acquired from a camera (e.g., the endoscope 121). Once the target is located, the tissue engager 106 can then be expanded to secure the device 100 in position (e.g., in the position shown in FIG. 1). Using images from the same or a different camera (e.g., the endoscope 121), the working channel 113_{L} can then be aligned with the target by moving the working channel 113_{L} to a deflected position (e.g., to the deflected position shown in FIG. 1). Once the working channel 113_{L} is aligned with the target, the target can be cannulated, for example by deploying a sheath 117 from the working channel 113_{L} into the target (e.g., into the Ampulla of Vater 123). Once the target is cannulated with the sheath 117, a guidewire 119 can be advanced into the target through the sheath 117. Once the guidewire 119 is in position, the third sheath 117 can be retracted. As this exemplary procedure demonstrates, securing the device 100 in position via the tissue engager 106 during cannulation (e.g., as shown in FIG. 1) can advantageously stabilize a visual field for the camera, can advantageously stabilize the camera, can advantageously stabilize the target relative to the device 100 so that the target can be in a fixed position while the working channel 113_{L} is moved to the position shown in FIG. 1, or any combination thereof. These various benefits can advantageously improve visualization of the target, cannulation of the target, or both. This can in turn result in decreased procedure times (e.g., from being able to cannulate the target more safely, reliably, and/or quickly). Such benefits can also improve the safety, reduce possible complications, and/or reduce the risks associated with cannulation procedures by enabling the target to be more reliably cannulated on the first attempt or by reducing the number of cannulation attempts that are required before the target is successfully cannulated.

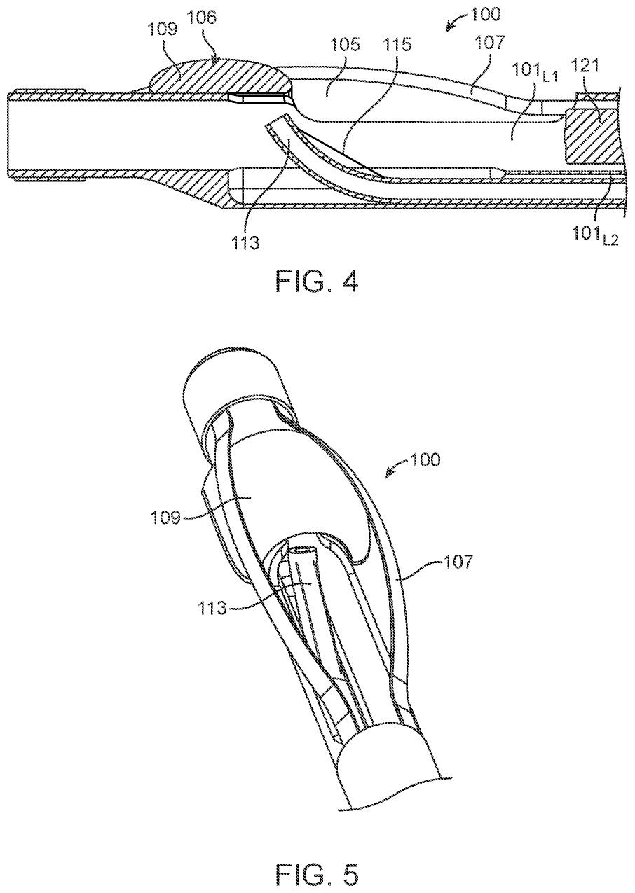

FIG. 1 illustrates that the device 100 can have one or multiple sheaths (also referred to as tubes or shafts), for example, 1 to 10 or more sheaths, including every 1 sheath increment within this range (e.g., 1 sheath, 2 sheaths, 10 sheaths). For example, FIG. 1 illustrates that the device 100 can have a first sheath 101, a second sheath 113, and a third sheath 117 (also referred to as sheath 101, sheath 113, and sheath 117, respectively, as first, second, and third tubes, respectively). The device 100 can have any combination of the sheath 101, the sheath 113, and the sheath 117. Each of the sheaths can have one or multiple lumens, for example, 1 to 10 or more lumens, including every 1 lumen increment within this range (e.g., 1 lumen, 2 lumens, 10 lumens). Each sheath can have the same or different number of lumens as another sheath. For example, FIG. 1 illustrates that the first sheath 101 can have one lumen (e.g., a first sheath lumen 101_{L}), the second sheath 113 can have one lumen (e.g., a second sheath lumen 113_{L}), and the third sheath 117 can have one lumen (e.g., a third sheath lumen 117_{L}). The second sheath lumen 113_{L} is also referred to as the working channel 113_{L}. As another example, the first sheath 101 can have two lumens (e.g., a first sheath first lumen 101_{L1} and a first sheath second lumen 101_{L2} as shown in FIG. 4), the second sheath 113 can have one lumen (e.g., the second sheath lumen 113_{L}), and the third sheath 117 can have one lumen (e.g., the third sheath lumen 117_{L}).

The second sheath 113 can be positionable in one or multiple lumens of the first sheath 101 (e.g., in the first sheath lumen 101_{L} of FIG. 1 or in the first sheath first lumen 101_{L1} and/or in the first sheath second lumen 101_{L2} of FIG. 4). The third sheath 117 can be positionable in a lumen of the second sheath (e.g., second sheath lumen 113_{L}) and/or in one or multiple lumens of the first sheath 101 (e.g., in the first sheath lumen 101_{L} of FIG. 1 or in the first sheath first lumen 101_{L1} and/or in the first sheath second lumen 101_{L2} of FIG. 4). The third sheath 117 can be concentrically inside the second sheath 113 and/or concentrically inside the first sheath 101. The second sheath 113 can be concentrically inside the first sheath 101. The second sheath 113 can be concentrically inside a lumen of the first sheath 101 (e.g., in the first sheath lumen 101_{L} of FIG. 1, in the first sheath first lumen 101_{L1} of FIG. 4, or in the first sheath second lumen 101_{L2} of FIG. 4).

The first sheath 101 can be, for example, a catheter (e.g., a steerable catheter, a non-steerable catheter). The second sheath 113 can be, for example, a working sheath (e.g., an articulatable sheath) such as a cannula. The third sheath 117 can be, for example, a papillotome (e.g., a standard papillotome).

The first, second, and third sheaths 101, 113, 117 can be moveable. The first, second, and third sheaths 101, 113, 117 can be bendable. The first, second, and third sheaths 101, 113, 117 can each have a straight configuration and multiple curved configurations. The first, second, and third sheaths 101, 113, 117 can be advanced to the target (e.g., the Ampulla of Vater 123).

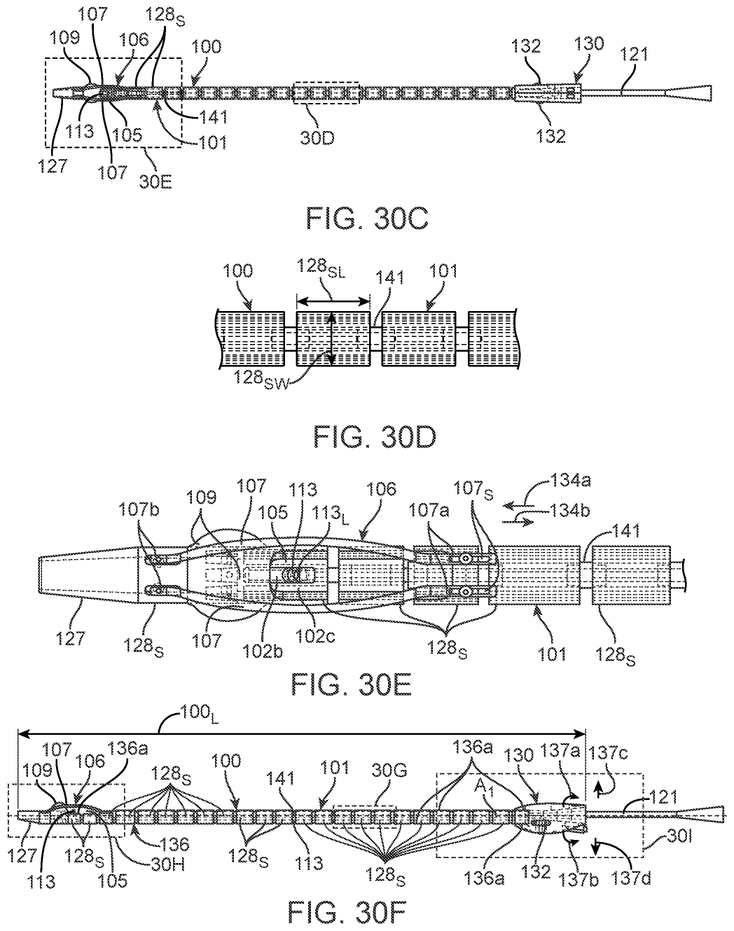

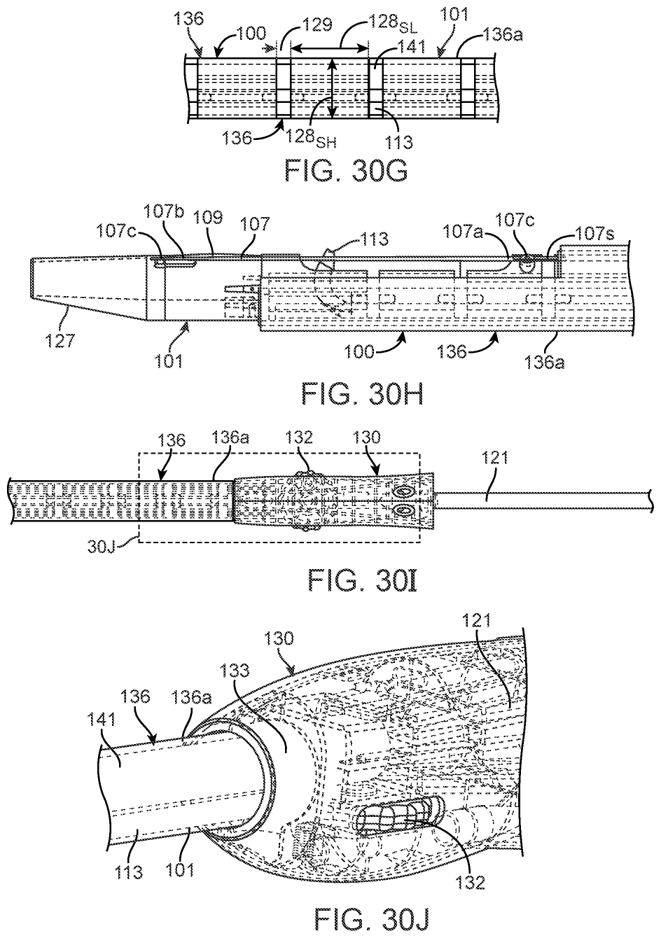

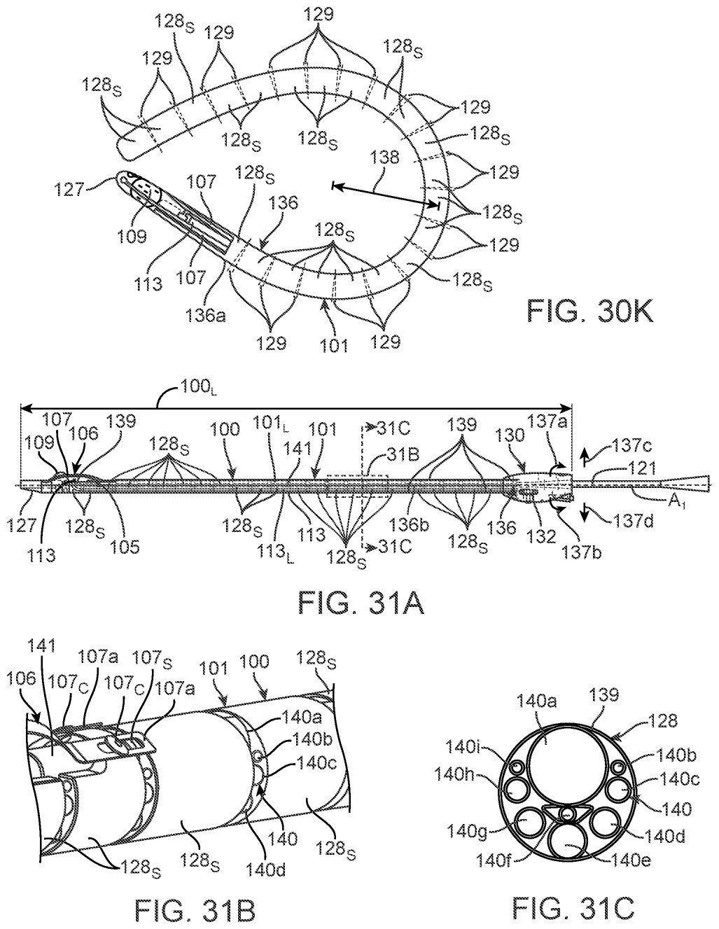

The first, second, and third sheaths 101, 113, 117 can be deflected into various curved configurations when the device 100 is being advanced to the target. The device 100 may or may not be steerable. Where the device 100 is steerable, the distal tip of the device 100 can be deflected to steer the device 100. For example, the device 100 can be steered by transmitting torque applied to a torque carrier 136 to the first sheath 101 (e.g., see FIGS. 30F-32E). As another example, when the device 100 is being steered to the target, the distal tip of the device 100 (e.g., the portion shown in FIG. 1) can be defected into various curved configurations, for example, by applying and releasing tension to pull wires connected to the distal tip of the device 100, so that the device 100 can be navigated through lumens in the body to the target. The pull wires can be, for example, connected to the first sheath 101 such that applying and releasing tension to the pull wires can deflect the distal tip of the first sheath 101 through lumens in the body to the target. As the device 100 is being steered to the target, the first, second, and third sheaths 101, 113, 117 can be deflected into various curved configurations. For example, deflecting the first sheath 101 can cause the second and third sheaths 113, 117 to deflect so that the first, second, and third sheaths 101, 113, 117 can be navigated at the same time through tortuous anatomy to the target. When the device 100 is being advanced to the target, the first, second, and third sheaths 101, 113, 117 can be moved together (e.g., in unison) to the target. As another example, the first sheath 101 can first be advanced to the target before the second and third sheaths 113, 117. Once the first sheath 101 is advanced to the target, the second and/or third sheaths 113, 117 can be advanced in a lumen of the first sheath 101 to the target. In such cases, the second and third sheaths 113, 117 can be flexible such that they can be advanced in a lumen of the first sheath and follow the curved path of the first sheath 101 to the target.

Where the device 100 is not steerable, the device 100 can be advanced to the target without any steering functionality. Where the device 100 is not steerable, the device 100 can be advanced to the target, for example, through a lumen in a steerable catheter that has already been advanced to or near the target. As another example, the device 100 can be advanced to the target, for example, over a guidewire. As still yet another example, the device 100 can be advanced to the target as-is, without the aid of any other devices or tools (e.g., guidewires).

Once the device 100 is at the target (e.g., the Ampulla of Vater), the first, second, and/or third sheaths 101, 113, and/or 117 can be deflected into various curved configurations to access the target (e.g., to cannulate the bile duct 124 or to cannulate the pancreatic duct 126). The curved configurations of the sheaths (e.g., the sheaths 101, 113, and 117) when the device is at the target can be different from the curved configurations that the sheaths (e.g., the sheaths 101, 113, and 117) are deflected into when the device 100 is being steered and/or advanced to the target during initial insertion. For example, when the device 100 is at the target, the second sheath 113 can be moveable to various working positions (also referred to as deflected positions and deflected configurations). FIG. 1 illustrates, for example, an exemplary working position of the second sheath 113. As shown in FIG. 1, when the second sheath 113 is in a working position, the distal end of the second sheath 113 can extend across (e.g., longitudinally across and/or transversely across) the lumen or lumens of the first sheath 101. The second sheath can be deflected into a working position (e.g., to the deflected position shown in FIG. 1) to align the working channel 113_{L} with the target. The working channel 113_{L} can be deflected to align the working channel 113_{L} with target. As explained above, once the working channel 113_{L} is aligned with the target, the target can be cannulated, for example by deploying the third sheath 117 from the working channel 113_{L} into the target (e.g., into the Ampulla of Vater 123).

FIG. 1 illustrates that the device 100 can have a guidewire 119. The guidewire 119 can be positionable in a lumen of the third sheath 117 (e.g., in the third sheath lumen 117_{L}), can be positionable in a lumen of the second sheath (e.g., in the second sheath lumen 113_{L}), can be positionable in one or multiple lumens of the first sheath 101 (e.g., in the first sheath lumen 101L of FIG. 1 or in the first sheath first lumen 101_{L1} and/or in the first sheath second lumen 101_{L2} of FIG. 4), or any combination thereof. The guidewire 119 can be translated longitudinally in the first sheath 101 (e.g., in the first sheath lumen 101_{L}), in the second sheath 113 (e.g., in the second sheath lumen 113_{L}), in the first sheath 101 (e.g., in the first sheath lumen 101_{L}), or any combination thereof. For example, when the second sheath 113 is aligned with the target (e.g., is in the deflected configuration shown in FIG. 1) and the third sheath 117 is advanced out of the second sheath 113 (e.g., to the position shown in FIG. 1), the guidewire 119 can be translated into the target (e.g., the Ampulla of Vater 123) and into any downstream passages (e.g., into the bile duct 124 or the pancreatic duct 126). As another example, while keeping the second sheath 113 aligned with the target (e.g., in the position shown in FIG. 1), the third sheath 117 can be advanced or extended further out of the second sheath 113 past the position shown in FIG. 1 and into the target. Once the third sheath 117 is advanced into the target, the guidewire 119 can be translated into the target (e.g., the Ampulla of Vater 123) and into any downstream passages (e.g., into the bile duct 124 or the pancreatic duct 126). As the arrangement in FIG. 1 shows, when the guidewire 119 is translated in the third sheath 119, the guidewire 119 can simultaneously move in the first, second, and third sheaths 101, 113, 117. The guidewire 119 can be translated independently of the first, second, and third sheaths 101, 113, 117. The guidewire 119 can be translated into and out (also referred to as advanced and withdrawn) of the target independently of the third sheath 117. The first sheath 101 can be kept in a secured position (e.g., via the tissue engager 106) while the guidewire 119 is advanced into the target. The second sheath 113 can be kept in an aligned position (e.g., the position shown in FIG. 1) while the guidewire 119 is advanced into the target. The third sheath 117 can be kept an aligned position (e.g., the position shown in FIG. 1) while the guidewire 119 is advanced into the target. The guidewire 119 can thus be moved independently of the first, second, and third sheaths 101, 113, 117. When the second sheath 113 is articulated to a deflected configuration, a distal end of the guidewire 119 can be curved. When the second sheath 113 is articulated to the deflected configuration, the distal end of the guidewire 119 can extend transversely across the lumen or lumens of the first sheath 101.

A camera attached to the device 100, an endoscope 121 inside or outside of the device 100, or both can visualize (e.g., capture images and/or video) the body space that the device 100 is in (e.g., the intestinal lumen 125), can visualize (e.g., capture images and/or video) of the target to be cannulated or operated on, or both. For example, FIG. 1 illustrates that the device 100 can have an endoscope 121. The endoscope 121 can be positionable in a lumen of a sheath, for example, in the first sheath lumen 101_{L}. The endoscope 121 can be moveable (e.g., translatable and/or rotatable) in the first sheath 101 (e.g., in the first sheath lumen 101_{L}). For example, the endoscope 121 can be translated longitudinally in a first direction 121a and in a second direction 121b within the device 100 (e.g., within the first sheath lumen 101_{L}). The first direction 121a can be toward a distal end of the device 100 and the second direction 121b can be toward a proximal end of the device 100. The first direction 121a can be opposite the second direction 121b such that the endoscope 121 can be moved forward (e.g., direction 121a) and backward (e.g., direction 121b) in the device 100. The endoscope 121 can be an end-viewing (e.g., front-viewing) endoscope, a side-viewing endoscope, or both. For example, FIG. 1 illustrates that the endoscope 121 can be an end-viewing (e.g., front viewing) endoscope. The endoscope 121 can be moved to any position in the first sheath 101. The endoscope 121 can capture images from any position in the first sheath 101, outside the first sheath 101, or both.

FIG. 1 illustrates that the device 100 can have an engager 106. The engager 106 can have, for example, zero, one, or multiple stabilizers 107, zero, one, or multiple expanders 109, or any combination thereof. The engager 106 can be a tissue engager. The engager 106 can engage with tissue. The engager 106 can contact tissue. The engager 106 can be a stabilizer. The engager 106 can stabilize the device 100 and/or a camera (e.g., the endoscope 121) in place in a lumen, for example, in the intestinal lumen 125 via the stabilizers 107 and/or expanders 109. The engager 106 can stabilize the device 100 and/or a camera (e.g., the endoscope 121) in place, for example, by forcing the stabilizers 107 and/or expanders 109 into tissue. For example, FIG. 1 illustrates that the engager 106 can stabilize the device 100 and/or the endoscope 121 in place in the intestinal lumen 125 by engaging with tissue 125_{T} that defines the intestinal lumen 125 with a stabilizer 107 and an expander 109.

The engager 106 can be expandable and contractible. The engager 106 can be an expandable and contractible cage, with the stabilizers 107 and/or the expanders 109 forming the cage. The engager 106 can have an unexpanded configuration, a fully expanded configuration, and any partially expanded configuration between the unexpanded configuration and the fully expanded configuration. For example, FIG. 1 illustrates the engager 106 in a fully expanded configuration. The engager 106 can be expanded, for example, by moving the stabilizers 107 and/or the expander 109 away from the device longitudinal axis A₁. The engager 106 can be contracted, for example, by moving the stabilizers 107 and/or the expander 109 toward the device longitudinal axis A₁.

When the engager 106 is in an expanded configuration (e.g., in the expanded configuration shown in FIG. 1) and in contact with the tissue of the lumen, the engager 106 can inhibit or prevent the first sheath 101 from translating and/or rotating in the lumen (e.g., in the intestinal lumen 125). This can advantageously allow the first sheath 101 to be secured or anchored in position while the second sheath 113 is deflected into alignment with the target (e.g., the Ampulla of Vater 123). As the engager 106 is expanded into an expanded configuration, the engager 106 can push the tissue defining the lumen (e.g., the tissue 125_{T}) away from the outer surface of the first sheath 101, away from the first sheath lumen 101, away from the device longitudinal axis A₁, away from the second sheath 113, or away from any combination thereof. This can advantageously create a space 105 for the second sheath 113 to be deflected into and can advantageously provide a larger visual field for the camera. When the first sheath 101 is anchored in the lumen via the engager 106, the endoscope 121 can be moveable in the first sheath lumen 101_{L} and can provide images of the second sheath 113 being deflected toward the target so that the user can determine when the second sheath 113 is aligned with the target.

The engager 106 can be expanded and contracted between any two configurations. For example, the engager 106 can be expanded from the unexpanded configuration to any partially expanded configuration or to the fully expanded configuration. The engager 106 can be expanded from a first partially expanded configuration to a second partially expanded configuration, where the second partially expanded configuration is more expanded than the first partially expanded configuration. The engager 106 can be expanded from any partially expanded configuration to the fully expanded configuration. The engager 106 can be contracted from any expanded configuration to any less expanded configuration, including to the unexpanded configuration.

FIG. 1 illustrates when the engager 106 is in an expanded configuration, the engager 106 (e.g., the stabilizers 107 and/or the expanders 109) can be pressed against the tissue defining the lumen (e.g., the intestinal lumen 125). FIG. 1 further illustrates that when the engager 106 is in an expanded configuration, the opposite side of the device 100 can be in contact with or be pressed into the tissue defining the lumen, for example, via the engager 106. For example, FIG. 1 illustrates that when the engager 106 is in an expanded configuration in which the device 100 is secured in the lumen (e.g., in the intestinal lumen 125), the engager 106 (e.g., the stabilizers 107 and/or the expanders 109) and the portion of the first sheath 101 opposite the engager 106 can contact the tissue defining the lumen (e.g., the tissue 125_{T}). As another example, when the engager 106 is in an expanded configuration, the opposite side of the device 100 (e.g., the side of the device 100 opposite the engager 106, for example, the outside of the first sheath 101) may not be in contact with or may not be pressed into the tissue defining the lumen.

FIG. 1 illustrates that when the engager 106 is in an expanded configuration, the engager 106 can define the space 105. As FIG. 1 shows, the space 105 can be between the tissue (e.g., the tissue 125_{T}) and the sheaths (e.g., the sheaths 101, 113, and/or 117). The space 105 can be between the stabilizers 107 and the sheaths (e.g., sheaths 101, 113, and/or 117). The space 105 can be between the expanders 109 and the sheaths (e.g., sheaths 101, 113, and/or 117). The space 105 can be between the stabilizers 107 and expanders 109 and the sheaths (e.g., sheaths 101, 113, and/or 117). As the engager 106 is expanded, the space 105 can be created and continue to become larger as the engager 106 continues to expand. As the engager 106 is contracted, the space 105 can become smaller. The space 105 can thereby be expanded and contracted. The space 105 can thus have an adjustable size (e.g., adjustable volume). For example, the size of the space 105 can be made larger and smaller by expanding and contracting the engager 106, respectively. The second sheath 113 can be deflected into space 105, for example, to align the second sheath 113 with the target (e.g., the Ampulla of Vater 123). For example, FIG. 1 illustrates the second sheath 113 in a deflected configuration in the space 105 in which the second sheath 113 is aligned with the target.

A camera (e.g., the endoscope 121), the second sheath 113, the third sheath 117, the guidewire 119, a tool deployable from the first sheath 101, a tool deployable from the second sheath 113, a tool deployable from the third sheath 117, or any combination thereof can be moveable in the space 105. For example, the space 105 can be a viewing window for a camera (e.g., the endoscope 121), for example, from inside the first sheath lumen 101_{L}. As another example, the space 105 can be a working space for one or multiple tools (e.g., the second sheath 113, the third sheath 117, and/or the guidewire 119). As yet another example, the space 105 can be a viewing window for the endoscope 121 and a working space for one or multiple tools (e.g., the second sheath 113, the third sheath 117, and/or the guidewire 119).

FIG. 1 illustrates that as the engager 106 is expanded into an expanded configuration (e.g., from an unexpanded configuration), the engager 106 can push the tissue defining the lumen (e.g., the tissue 125_{T}) away from the device longitudinal axis A₁ to create the space 105. FIG. 1 further illustrates that the second sheath 113 can be deflected into the space 105 to align the second sheath lumen 113_{L} with the target (e.g., the Ampulla of Vater 123). This movement of the second sheath 113 can be visualized with the camera (e.g., the endoscope 121) so that the operator can determine when the second sheath 113 is aligned with the target. The endoscope 121 can be moveable in the first sheath lumen 101_{L} and/or the space 105 while the second sheath 113 being deflected toward and aligned with the target so that multiple viewing angles of the target and the second sheath 113 are possible.

FIG. 1 illustrates that the space 105 can include the portion of the first sheath lumen 101_{L} that extends between a first longitudinal end of the engager 106 and a second longitudinal end of the engager 106. The space 105 can include the portion of the first sheath lumen 101_{L} that extends between a first longitudinal terminal end of the engager 106 and a second longitudinal terminal end of the engager 106. The first longitudinal terminal end of the engager 106 can be proximal the second longitudinal terminal end of the engager 106. For example, FIG. 1 illustrates that the first longitudinal terminal end of the engager 106 can be closer to the camera (e.g., the endoscope 121) than the second longitudinal terminal end of the engager 106. FIG. 1 further illustrates that the first longitudinal terminal end of the engager 106 can be in the same longitudinal position as the camera (e.g., as the endoscope 121).

FIG. 1 illustrates that the engager 106 can have a stabilizer 107. The engager 106 can have one or multiple stabilizers 107, for example, 1 to 10 or more stabilizers 107, including every 1 stabilizer 107 increment within this range (e.g., 1 stabilizer, 2 stabilizers, 10 stabilizers). The stabilizers 107 can be struts. The stabilizers 107 can be ribbons. The stabilizers 107 can be flexible, inflexible, or both. The stabilizers 107 can be elastic, inelastic, or both. The stabilizers 107 can be metal. The stabilizers 107 can be plastic. The stabilizers 107 can be expandable, contractible, or both. The stabilizers 107 can be expanded, contracted, or both. The stabilizers 107 can have a length, for example, of about 10 mm to about 100 mm, or more narrowly from about 10 mm to about 70 mm, or narrowly from about 10 mm to about 50 mm, including every 1 mm increment within these ranges (e.g., 10 mm, 30 mm, 50 mm, 70 mm, 100 mm). The stabilizers 107 can stabilize the device 100 (e.g., in the intestinal lumen 125), for example, by contacting tissue, and can facilitate improved visualization of the mucosa in the lumen, for example, by defining the space 105 between the tissue and the sheaths (e.g., sheaths 101, 113, and/or 117). The stabilizers 107 can define the space 105, for example, when the stabilizers 107 are in an expanded configuration.

Each stabilizer 107 can be moveable from a stabilizer first position to a stabilizer second position and to any stabilizer position between the stabilizer first and second positions such that the stabilizer 107 is farther from a device longitudinal axis A₁ when the stabilizer 107 is in the stabilizer second position than when the stabilizer 107 is in the stabilizer first position. When the engager 106 is in the unexpanded configuration, the stabilizers 107 can be in the stabilizer first position. When the engager 106 is in the fully expanded configuration, the stabilizers 107 can be in the stabilizer second position. When the engager 106 is in a partially expanded configuration, the stabilizers 107 can be in a position (also referred to as a stabilizer third position) between the stabilizer first and second positions. For example, FIG. 1 illustrates a stabilizer 107 in a stabilizer second position. When a stabilizer 107 is in an expanded or extended configuration (e.g., stabilizer second or third positions), an outer surface of the stabilizer 107 (e.g., the medial portion of the stabilizer 107) can be farther from the device longitudinal axis A₁ than when the stabilizer 107 is in an unexpanded configuration (e.g., stabilizer first position). When a stabilizer 107 is in an expanded or extended configuration (e.g., stabilizer second or third position), the medial portion of the stabilizer 107 can be farther from the device longitudinal axis A₁ than a proximal portion of the stabilizer 107 and/or can be farther from the device longitudinal axis A₁ than a distal portion of the stabilizer 107. For example, FIG. 1 illustrates that when the stabilizer 107 is in a fully expanded or extended configuration, the medial portion of the stabilizer 107 can be farther from the device longitudinal axis A₁ than a proximal portion of the stabilizer 107 and can be farther from the device longitudinal axis A₁ than a distal portion of the stabilizer 107.

When the device 100 has multiple stabilizers 107, the stabilizers 107 can be individually and/or collectively moved or changed from the stabilizer first position to the stabilizer second position to move (e.g., expand, extend) the stabilizers 107 away from (e.g., radially away from) the device longitudinal axis A₁ and vice versa. For example, the device 100 can have a stabilizer actuator (e.g., a control on a device handle) that can be used to actuate the stabilizers 107 individually and/or collectively. A tether (e.g., wire, rod) can connect the control on the device handle to the stabilizers 107 or to a connector to which the stabilizers 107 are coupled to. Applying tension to the tether can expand the stabilizers 107, for example, by pulling the distal ends of the stabilizers 107 closer to the proximal ends of the stabilizers 107. As another example, one or more of the expanders 109 can move the stabilizers 107 inward and outward to contract and expand the stabilizers 107, respectively. In such cases, the expanders 109 that expand the stabilizers 107 outward can be the stabilizer actuators. FIG. 1 illustrates, for example, that the expander 109 can move the stabilizers 107 from the stabilizer first positions to the stabilizer second position, or to any stabilizer third position. The expander 109 can move the stabilizers 107 from any stabilizer third position to the stabilizer second position. When the expander 109 is contracted (e.g., when its size is reduced, for example, from deflation), the contraction of the expander 109 can cause the stabilizers 107 to contract. The expander 109 can move the stabilizers 107 from the stabilizer second position to any stabilizer third position or to the stabilizer first position. The expander 109 can move the stabilizers 107 from any stabilizer third position to the stabilizer first position.

FIG. 1 illustrates that the engager 106 can have an expander 109. The engager 106 can have one or multiple expanders 109, for example, 1 to 10 or more expanders 109, including every 1 expander 109 increment within this range (e.g., 1 expander, 2 expanders, 10 expanders). The expanders 109 can be expandable, contractible, or both. The expanders 109 can be expanded, contracted, or both. FIG. 1 illustrates that the engager 106 can have one expander 109. The expander 109 can be, for example, a balloon. As another example, the expander 109 can be a stent. The expander 109 can be inflatable and deflatable. The expander 109 can expand when inflated. The expander 109 can contract when deflated. The expanders 109 can be flexible, inflexible, or both. The expanders 109 can be elastic, inelastic, or both. For example, FIG. 1 illustrates that the expanders 109 can be balloons. The expanders 109 can stabilize the device 100 (e.g., in the intestinal lumen 125), for example, by contacting tissue, and can facilitate improved visualization of the mucosa in the lumen, for example, by defining the space 105 between the tissue and the sheaths (e.g., sheaths 101, 113, and/or 117). The expanders 109 can define the space 105, for example, when the expanders 109 are in an expanded configuration (e.g., when they are inflated). For variations in which the expanders 109 do not contact tissue when expanded, the expanders 109 can stabilize the device 100 (e.g., in the intestinal lumen 125), for example, by causing the stabilizers 107 to contact tissue. Thus, when the stabilizers 107 are engaged with tissue (e.g., the tissue 125_{T}), the expanders 109 may or may not be engaged with tissue (e.g., the tissue 125_{T}).

Each expander 109 can be moveable from an expander first configuration to an expander second configuration and to any expander configuration between the expander first and second configurations such that the expander 109 (e.g., an outer surface of the expander 109) is farther from a device longitudinal axis A₁ when the expander 109 is in the expander second configuration than when the expander 109 is in the expander first configuration. When the engager 106 is in the unexpanded configuration, the expanders 109 can be in the expander first configuration. When the engager 106 is in the fully expanded configuration, the expanders 109 can be in the expander second configuration. When the engager 106 is in a partially expanded configuration, the expanders 109 can be in a configuration (also referred to as an expander third configuration) between the expander first and second configurations. For example, FIG. 1 illustrates an expander 109 in an expander second configuration. When an expander 109 is in an expanded or extended configuration (e.g., expander second or third configurations), an outer surface of the expander 109 (e.g., the medial portion of the expander 109) can be farther from the device longitudinal axis A₁ than when the expander 109 is in an unexpanded configuration (e.g., expander first configuration). When an expander 109 is in an expanded or extended configuration (e.g., expander second or third configuration), the medial portion of the expander 109 can be farther from the device longitudinal axis A₁ than a proximal portion of the expander 109 and/or can be farther from the device longitudinal axis A₁ than a distal portion of the expander 109. For example, FIG. 1 illustrates that when the expander 109 is in a fully expanded or extended configuration, a medial portion of the outer surface of the expander 109 can be farther from the device longitudinal axis A₁ than a proximal portion of the expander 109 and can be farther from the device longitudinal axis A₁ than a distal portion of the expander 109.

When the device 100 has multiple expanders 109, the expanders 109 can be individually and/or collectively moved or changed from the expander first configuration to the expander second configuration to move (e.g., expand, extend) the expanders 109 away from (e.g., radially away from) the device longitudinal axis A₁ and vice versa. For example, the device 100 can have an expander actuator (e.g., an inflation lumen) that can be used to actuate the expanders 109 individually and/or collectively. When the expanders 109 are balloons, inflating the expanders 109 can expand the expanders 109 and deflating the expanders 109 can contract the expanders 109.

For example, the device 100 can have a lumen to expand (e.g., inflate) and contract (e.g., deflate) the expander 109. As another example, the device can have two lumens in fluid communication with each expander 109. For example, a lumen of the first sheath 101 can be an expander lumen that is connected to the expander 109 that can be used to inflate and deflate the expander 109. Fluid (e.g., gas, liquid) can be sent through the expander lumen and into the expander 109 to expand the expander 109. Fluid can be removed from the expander 109 via the expander lumen to contract the expander 109. A control on the device handle can be used to control the expansion and contraction of the expander 109.

The expander 109 can be stabilizer actuator. The expander 109 can actuate the stabilizers 107. The expander 109 can move the stabilizers 107 outward, for example, away from the first sheath 101 and/or away from the device longitudinal axis A₁. The expander 109 can move the stabilizers 107 inward, for example, toward the first sheath 101 and/or toward the device longitudinal axis A₁. The expander 109 can expand the stabilizers 107. The expander 109 can contract the stabilizers 107. The expander 109 can expand and contract the stabilizers 107. For example, the expander 109 can move the stabilizers 107 from a stabilizer first position (e.g., a non-expanded position) to a stabilizer second position (e.g., an expanded position) by being expanded from an unexpanded configuration to an expanded configuration. FIG. 1 illustrates the expander 109 in an expanded configuration (e.g., in a fully expanded configuration), whereby the expander 109 pushed the stabilizer 107 away from the device longitudinal axis A₁ when the expander 109 (e.g., balloon) was inflated to the expanded configuration shown in FIG. 1. The expander 109 can push the stabilizers 107 radially outward. When the expander 109 is contracted (e.g., when its size is reduced, for example, from deflation), the contraction of the expander 109 can cause the stabilizers 107 to contract. For example, the stabilizers 107 can be biased to return to a less expanded configuration (e.g., the stabilizer first position) such that when the expander 109 is contracted the stabilizers 107 can naturally contract to a contracted configuration (e.g., to a stabilizer third position or to the stabilizer first position, depending on how much the expander 109 is contracted). As the expander 109 is contracted (e.g., deflated), the stabilizers 107 can contract toward the device longitudinal axis A₁ at the same or a different rate (e.g., speed) as an outer surface of the expander 109. As another example, when the expander 109 is contracted, the expander 109 can contract the stabilizers 107 by pulling the stabilizers 107 to a contracted configuration (e.g., by pulling the stabilizers 107 back to a stabilizer third position to all the way back to the stabilizer first position). In such cases the stabilizers 107 may or may not be biased to return to a less expanded configuration. The expander 109 can pull the stabilizers 107 radially inward. Thus, the stabilizers 107 can be moved away from and toward the device longitudinal axis A₁ with or without an expander 109, and/or with or without assistance from an expander 109.

The expander 109 can have an expander dimension (e.g., diameter, width, length). The expander dimension of the expander 109 can be selectively controlled by the operator. Where the expander dimension is a width or a length, the width or the length can be the total width or the total length of the expander 109 in whichever configuration the expander 109 is in. For example, when the expander 109 is in a fully expanded configuration, the expander 109 the expander dimension (e.g., diameter, width, and/or length) can be from about 10 mm to about 80 mm, including every 1 mm increment within this range (e.g., 10 mm, 20 mm, 40 mm, 60 mm, 80 mm). For example, when the expander 109 is a balloon and the balloon is in a fully inflated configuration, the balloon can have a diameter, width, and/or length from about 10 mm to about 80 mm, including every 1 mm increment within this range (e.g., 10 mm, 20 mm, 40 mm, 60 mm, 80 mm). The dimension (e.g., diameter) of the balloon can be selectively controlled by the operator. Different balloon sizes and shapes can be selected, for example, based on the size and weight of the patient, the presence or absence of altered anatomy, the procedure to be performed (e.g., ERCP) or any combination thereof. For example, when the device 100 is used for ERCP procedures, the expander (e.g., balloon) can have a dimension (e.g., diameter) of about 20 mm to about 40 mm, including every 1 mm increment within this range.

The expander 109 (e.g., balloon) can be placed on the distal portion of the device 100 as shown in FIG. 1 and/or along the stabilizers 107 to provide a skirt, as such configurations can desirably inhibit or prevent mucosal folds from entering the space 105. For example, FIG. 1 illustrates that the expander 109 can be distal of the space 105. As another example, the expander 109 can be proximal the space 105. As yet another example, the device 100 can have a first expander 109 (e.g., first balloon) distal the space 105 and a second expander 109 (e.g., second balloon) proximal the space 105 with or without the stabilizers 107 (e.g., with or without the stabilizers 107 shown in FIG. 1).

FIG. 1 thereby shows (1) that the stabilizers 107 can be actuated independently of the expanders 109, and vice versa, (2) that the stabilizers 107 can be actuated by one or multiple expanders 109 (e.g., by one or multiple expanders 109 pushing the stabilizers 107 away from the device longitudinal axis A₁ when the expanders 109 are actuated, for example, when the expanders 109 are inflated), (3) that the expanders 109 can be actuated by one or multiple stabilizers 107 (e.g., by one or multiple stabilizers 107 pulling the expanders 109 away from the device longitudinal axis A₁ when the stabilizers 107 are actuated, for example, when the stabilizers 107 are expanded), or any combination thereof. When the engager 106 is expanded (e.g., by expanding the stabilizers 107 and/or the expanders 109), the engager 106 can engage with the tissue. As the engager 106 is expanded, the engager 106 can push tissue (e.g., the tissue 125_{T}) away from the device 100 (e.g., away from the device longitudinal axis A₁ and/or away from the first sheath 101). Pushing the tissue away from the device 100 can unfold, de-wrinkle, and/or stretch the tissue defining the lumen wall (e.g., the tissue 125_{T}) if the tissue has folds and/or wrinkles, which can in turn make the target (e.g., the Ampulla of Vater 123) easier to see and/or easier to cannulate.

FIG. 1 illustrates that the engager 106 can be expanded such that the distal tip of the device 100 extends across the entire lumen (e.g., the intestinal lumen 125), with the engager 106 in contact with tissue on a first side of the lumen and with the first sheath 101 in contact with tissue on a second side of the lumen. In such cases, the device 100 can be secured in position by expanding the engager 106 so that the engager 106 and the first sheath 101 are pressed against the tissue (e.g., the tissue 125_{T}). As another example, the engager 106 can be expanded such that the distal tip of the device 100 does not extend across the entire lumen (e.g., the intestinal lumen 125) such that the engager 106 is in contact with tissue on a first side of the lumen but such that the portion of the first sheath 101 opposite the engager 106 is not in contact with tissue on a second side of the lumen. In such cases, the device 100 can create the space 105 and be secured in position, for example, via the user holding the device 100 in position and/or by the engager 106 grabbing onto tissue (e.g., by pinching it, suctioning to it).

FIG. 1 illustrates that the stabilizers 107 and/or the expanders 109 of the engager 106 can thereby stabilize the device 100 (e.g., in the intestinal lumen 125), for example, by contacting tissue, and can advantageously facilitate improved visualization of the mucosa in the lumen (e.g., the intestinal lumen 125), for example, by creating the space 105 between the tissue and the sheaths (e.g., sheaths 101, 113, and/or 117) when the stabilizers 107 and/or the expanders 109 are in an expanded configuration.

FIG. 1 illustrates that when the stabilizers 107 are in the stabilizer second position, the stabilizers 107 can be positioned (e.g., extended or expanded) outward, away from the device longitudinal axis A₁. Each stabilizer 107 can be expanded outward (e.g., radially outward). For example, when there are multiple stabilizers 107 (e.g., two stabilizers 107), the all the stabilizers 107 (e.g., the two stabilizers 107) can be expanded the same distance outward. The motion of the stabilizers 107 outward can be uniform (e.g., in straight expansion planes), for example, in planes that intersect the device longitudinal axis A₁. For example, each stabilizer 107 can expand outward in a single plane that intersects with the device longitudinal axis A₁. For example, in some variations of the device 100, the stabilizer 107 shown in FIG. 1 has been expanded radially outward). As another example, the stabilizers 107 can move away from each other as the stabilizers 107 expand outward. Such movement of the stabilizers 1007 away from each other can advantageously assist with unfolding, de-wrinkling, and/or stretching the tissue defining the lumen wall (e.g., the tissue 125_{T}) if the tissue has folds and/or wrinkles, which can in turn make the target (e.g., the Ampulla of Vater 123) easier to see and/or easier to cannulate. For example, the motion of the stabilizers 107 outward can have a secondary actuation direction allowing parting of the stabilizers 107 to occur such that the stabilizers 107 can move away from each other relative to a transverse axial plane (e.g., in a plane that intersects the device longitudinal axis A₁ and bisects the expansion planes of the stabilizers 107 in FIG. 2), whereby the stabilizers 107 can move outward in a curved plane or in multiple planes (e.g., in a first plane that intersects with the device longitudinal axis A₁ and in a second plane that does not intersect with the device longitudinal axis A₁). As yet another example, the stabilizers 107 can move toward each other as the stabilizers 107 expand outward. This may or may not pinch tissue. When tissue is pinched, this can assist with securing the device 100 in the lumen (e.g., in the intestinal lumen 125). As another example, the pinching of the tissue can secure the device 100 in the lumen.

FIG. 1 illustrates that the stabilizers 107 can be attached to or integrated with the first sheath 101. The stabilizers 107 can be embedded in the wall of the first sheath 101. For example, a distal end of the stabilizers 107 can be attached to the first sheath 101 beyond a first side of the space 105 and a proximal end of the stabilizers 107 can be attached to the first sheath beyond a second side of the space 105. As another example, a first end of the stabilizers 107 (e.g., the proximal end) can be attached to the first sheath 101 at the proximal edge of the space 105 and a second end of the stabilizers 107 (e.g., the distal end) can be attached to the first sheath 101 at the distal edge of the space 105. As another example, one or both ends of the stabilizers 107 can be attached to couplers that are attached to or integrated with the first sheath 101. For example, FIG. 1 illustrates that a distal end of the stabilizers 107 can be attached to a distal coupler 110. FIG. 1 illustrates that the stabilizers 107 can extend over the space 105, from a first side of the space 105 (e.g., a proximal side) to a second side of the space 105 (e.g., a distal side).

FIG. 1 illustrates that the stabilizers 107 can have a stabilizer thickness 107_{T}. The stabilizer thickness 107_{T} can be about 0.005 inches to about 0.250 inches thick, including every 0.001 inches within this range (e.g., 0.005 inches, 0.100 inches, 0.250 inches), but the stabilizer thickness 107_{T} may be sized to accommodate any anatomy, for example, altered anatomy, such that dimensions outside of this range are anticipated and dependent on the anatomy into which the device 100 is placed.

When the engager 106 is in the unexpanded configuration or a partially expanded configuration, the engager 106 can be moveable (e.g., longitudinally translatable and/or rotatable) in the lumen (e.g., in the intestinal lumen 125) to position the device 100 in the lumen, for example, to perform an ERCP procedure. For example, for variations in which the engager 106 is attached to the first sheath 101, the engager 106 can be moved by moving (e.g., translating, rotating, deflecting) the first sheath 101. In such cases, when the engager 106 is in the unexpanded configuration or is in a partially expanded configuration in which the engager 106 is not secured in the lumen, the engager 106 can be can be translated in directions 121a and 121b in the lumen, for example, by translating the sheath 101 in directions 121a and 121b in the lumen. As another example, when the engager 106 is in the unexpanded configuration or is in a partially expanded configuration in which the engager 106 is not secured in the lumen, the engager 106 can be can be rotated in the lumen, for example, by rotating the first sheath 101 about the device longitudinal axis A₁ in the lumen (e.g., for variations in which the engager 106 is attached to the first sheath 101, as shown, for example, in FIG. 1). As still yet another example, where the distal tip of the first sheath 101 is deflectable (e.g., in steerable variations of the device 100 where the distal tip of the first sheath 101 is deflectable to steer the device 100 to the target), the engager 106 can be moved in the lumen, for example, by deflecting the distal tip of the first sheath 101 (e.g., by applying and releasing tension to pull wires attached to the distal tip of the first sheath 101). When the engager 106 is in an unexpanded configuration or a partially expanded configuration, the position of the engager 106 relative to the target can be variably adjusted or optimized by translating, rotating, and/or deflecting the first sheath 101. Deflecting the first sheath 101 (e.g., with the steering controls) can change position of the second sheath 113 relative to the target when the second sheath 113 is in a deflected configuration and can thereby assist in aligning the second sheath 113 with the target. When the engager 106 is in the fully expanded configuration (e.g., as shown in FIG. 1) or is in a partially expanded configuration in which the engager 106 is secured in the lumen, the engager 106 can stabilize the device 100 in the lumen (e.g., in the intestinal lumen 125) such that movement of the device 100 in the lumen is inhibited or prevented. When the engager is in a fully expanded configuration or is in a partially expanded configuration in which the engager 106 is secured in the lumen, translational and/or rotational movement of the engager 106 can be inhibited or prevented by the engagement of the engager 106 with the tissue defining the lumen, but in such cases, the position of the engager 106 relative to the target can be variably adjusted or optimized while the first sheath 101 is in a secured position by deflecting the first sheath 101 (e.g., using the steering controls) in embodiments where the first sheath 101 is deflectable.

FIG. 1 illustrates that the stabilizers 107 can have an outer surface 107_{OS} and an inner surface 107_{IS}. The stabilizer outer surface 107_{OS} can have surface features that permit increased mucosal gripping. Such surface features can include any gripper, for example, gecko feet, suction cups, ribs, or any combination thereof. As another example, the stabilizer outer surface 107_{OS} can have suction ports on the struts to apply negative pressure onto the mucosal surface. When negative pressure is applied to the mucosal surface (e.g., tissue 125_{T}) through the suction ports of the stabilizers 107, the gripping force of the stabilizers 107 can be increased. The stabilizers 107 can have suction lumens that terminate at the suction ports that are connected to a negative pressure source (e.g., a pump). The stabilizer inner surface 107_{IS} can have one or multiple markings 111, for example, 1 to 5 or more markings 11, including every 1 marking 111 increment within this range (e.g., 1 marking, 2 markings, 5 markings). FIG. 1 illustrates that each stabilizer 107 can have one marking 111. The markings 111 can be alignment markings. For example, the markings 111 can demarcate the position of the second sheath 113 and/or the third sheath 117 when the device 100 is in a fully deployed configuration (e.g., when the second sheath 113 is aligned with the target), thus providing a targeting system for the operator of the device 100 that can advantageously increase the efficiency of cannulation using the device 100. The markings 111 can be seen, for example, on the images that the camera (e.g., the endoscope 121) can provide during a procedure so that the operator can determine, for example, when the second sheath 113 is aligned with the target (e.g., the Ampulla of Vater 123). For example, the device 100 can advantageously decrease the total time of an ERCP procedure compared to current devices used for ERCP procedures by half or by more than half. The use of the grippers (e.g., surface features, suction ports, or both) and/or the markings 111 can be aided via a computer assisted targeting system. For example, the device 100 can allow efficient data management and traceability (e.g., using blockchain technology).

The entire device 100, or parts of the device 100 can be coated with a lubricious coating to permit easy transport of the device to the area of interest. For example, the stabilizers 107 and/or the expanders 109 can be coated with a surface coating (e.g., with fluoropolymers such as polytetrafluoroethylene, for example, Teflon®) that can minimize mucosal damage when the stabilizers 107 and/or the expanders 109 contact and move against mucosal surfaces (e.g., tissue 125_{T}).

During ERCP procedures, fluoroscopic radiological guidance is typically used, for example, especially once the Ampulla of Vater 123 is cannulated and tools are passed into the biliary tree (e.g., the bile duct 124 or the pancreatic duct 126, also referred to as branches 124 and 126). During ERCP procedures, fluoroscopic radiological guidance can be used, for example, once the Ampulla of Vater 123 is cannulated and tools are passed into the biliary tree (e.g., the bile duct 124 or the pancreatic duct 126, also referred to as branches 124 and 126). In this regard, the device 100 can have features permitting 3-dimensional appreciation of the position of the device 100 on a 2-D image. Such features can include, for example, radiopaque markings and notches. In such cases, the materials used in the device 100 can be magnetically optimized to permit external control and/or stabilization of the device 100 via magnets. To facilitate radiological visualization of the device 100 and for other uses one or more irrigation ports may exist on the device 100. The radiopaque markings and notches can be the same as or different from the markings 111.

FIG. 1 illustrates that when the engager 106 is in the fully expanded configuration, an expanded dimension D_{E} of the space 105 as measured from the peak of a stabilizer 107 to the device longitudinal axis A₁ can be about 10 mm to about 60 mm, including every 1 mm increment within this range (e.g., 10 mm, 30 mm, 60 mm). The peak of the stabilizer 107 when determining the expanded dimension D_{E} can be the point on the stabilizer 107 that is furthest from the device longitudinal axis A₁ when the engager 106 is in the fully expanded configuration. The engager 106 can be expanded based on the size of the lumen the engager is in. For example, to stabilize the device 100 in a lumen (e.g., the intestinal lumen 125), the engager 106 can be expanded so that the space 105 has the expanded dimension D_{E}.

FIG. 1 illustrates that the first sheath 101 can have one or multiple openings 102, for example, a first opening 102a, a second opening 102b, and a third opening 102c, or any combination thereof. The first opening 102a can be the distal terminal opening of the first sheath lumen (e.g., lumen 101_{L} or lumen 101_{L1}). The first opening 102a can be the distal most opening in the first sheath 101. For example, the first opening 102a can be distal the second and third openings 102b, 102c. The second and third openings 102b, 103b can be proximal the first opening 102a. The second opening 102b can be opposite (e.g., diametrically opposite) the third opening 102c. The space 105 can include the third opening 102c. The distal terminal end of the endoscope 121 can be positioned in any position relative to the openings 102, for example, distal the first opening 102a, in the first opening 102a, proximal the first opening 102a, proximal the second opening 102b, between the second and third openings 102b, 102c, proximal the second and third openings 102b, 102c, or any combination thereof.

The endoscope 121 can capture images from any position from inside or outside the first sheath 101. For example, the endoscope 121 can capture images of the lumen (e.g., the intestinal lumen 125), such as images of the target (e.g., the Ampulla of Vater 123), when the distal terminal end of the endoscope 121 is distal the first opening 102a. As another example, the endoscope 121 can capture images of the lumen, such as images of the target (e.g., the Ampulla of Vater 123), when the distal terminal end of the endoscope 121 is proximal the third opening 102c, for example, in the position shown in FIG. 1.

FIG. 1 illustrates that the endoscope 121 can be moveable through first opening 102a. For example, the endoscope 121 can be moved through (e.g., out of and back into) the first opening 102a, for example, along directions 121a and 121b, respectively. When the device 100 is being steered to the target (e.g., to the Ampulla of Vater 123), the endoscope 121 can be in any position. For example, when the device 100 is being steered to the target (e.g., to the Ampulla of Vater 123), the endoscope 121 can extend through the first opening 102a. In such a case, the first opening 102a can allow the endoscope 121 to be a distal terminal end of the device 100 while the device 100 is being steered to the target, such as a target cannulation location (e.g., to the Ampulla of Vater 123), to provide the operator with vision of the lumen through which the device 100 is being steered. When the device 100 is at the target (e.g., to the Ampulla of Vater 123), the endoscope 121 can be in any position. For example, when the device 100 is at the target (e.g., the Ampulla of Vater 123), the endoscope 121 can be in the position shown in FIG. 1 to provide the operator with vision of the target. For example, for variations in which the distal terminal end of the endoscope 121 is distal the first opening 102a during steering, after the device 100 is steered to the target, the endoscope 121 can be retracted into the first sheath 101, for example, to the position shown in FIG. 1, to provide the operator with vision of the target cannulation location (e.g., the Ampulla of Vater 123) during cannulation thereof.

The endoscope 121 can capture images through any of the openings 102, for example, through the first opening 102a, through the second opening 102b, through the third opening 102c, or any combination thereof. For example, in the position shown in FIG. 1, the endoscope 121 can capture images through the third opening 102c. In the position shown in FIG. 1, the endoscope 121 can capture images of the target (e.g., Ampulla of Vater 123) through the third opening 102c.

FIG. 1 illustrates that the second sheath 113, the third sheath 117, and/or the guidewire 119 can be moveable in the second opening 102b, can be moveable in the first sheath lumen (e.g., lumen 101_{L} or lumen 101_{L1}), can be moveable in the space 105, can be moveable outside of the device 100 beyond the space 105, or any combination thereof.

The second opening 102b can allow the second sheath 113, the third sheath 117, and/or the guidewire 119 to be moved into the first sheath lumen (e.g., lumen 101_{L} or lumen 101_{L1}), into the space 105, or into both, for example, from a non-deflected configuration into the arrangement shown in FIG. 1. The second opening 102b can allow the second sheath 113, the third sheath 117, and/or the guidewire 119 to be moved into and out of the first sheath lumen (e.g., lumen 101L or lumen 101_{L1}), into and out of the space 105, and/or into and out of the target (e.g., to cannulate the target), or any combination thereof, for example, from a non-deflected configuration to the deflected configuration shown in FIG. 1. For example, FIG. 1 illustrates that the second opening 102b can allow the second sheath 113, the third sheath 117, and/or the guidewire 119 to extend into the first sheath lumen (e.g., lumen 101_{L} or lumen 101_{L1}) so that the second sheath 113, the third sheath 117, and the guidewire 119 can be moved into and out of the space 105.

The third opening 102c can allow the second sheath 113, the third sheath 117, and the guidewire 119 to extend into the space 105 or to extend further into the space 105 (e.g., for cases in which the first sheath lumen 101_{L} is considered part of the space 105), past the first sheath lumen (e.g., lumen 101_{L} or lumen 101_{L1}), and away from the device longitudinal axis A₁.

FIG. 1 illustrates that the second sheath 113 can be positionable in the space 105. For example, FIG. 1 illustrates that the second sheath 113 can be positionable in the space 105 at an angle 114. The angle 114 can be adjustable, for example, by deflecting the second sheath 113 toward and away from the target (e.g., the Ampulla of Vater 123). For example, FIG. 1 illustrates that the second sheath 113 can be positionable in the space 105 such that the second sheath 113 is at the angle 114 relative to the target location (e.g., the Ampulla of Vater 123, also referred to as the target). The angle 114 can advantageously facilitate cannulation of the target location by aligning the exit port of the second sheath 113 toward the target location so that the second sheath longitudinal axis A₂ intersects with and is aligned with the target location, such as with a lumen of the target location. The target location can be, for example, the Ampulla of Vater 123 such that when the second sheath 113 is at the angle 114, the second sheath longitudinal axis A₂ is aligned with longitudinal axis of the Ampulla of Vater 123. As another example, the target location can be the Ampulla of Vater 123 such that when the second sheath 113 is at the angle 114, the second sheath longitudinal axis A₂ is aligned with longitudinal axis of the Ampulla of Vater 123, plus or minus 1 to 10 degrees, including every 1 degree increment within this range.

The angle 114 can be the angle between the device longitudinal axis A₁ and the second sheath longitudinal axis A₂. The second sheath longitudinal axis A₂ can have a first portion A₂a and a second portion A_{2b}. The first portion A₂ₐ of the second sheath longitudinal axis A₂ can be the portion of the second sheath longitudinal axis A₂ that extends out of the exit port of the second sheath 113 toward the target location. The first portion A₂a of the second sheath longitudinal axis A₂ can be a distal portion of a center longitudinal axis of the second sheath lumen 113_{L}. The second portion A_{2b} of the second sheath longitudinal axis A₂ can be a proximal portion of the center longitudinal axis of the second sheath lumen 113_{L}. The exit port of the second sheath 113 can be the distal terminal opening in the second sheath 113. The first portion A₂a of the second sheath longitudinal axis A₂ can be perpendicular to the exit port. The angle 114 can be the angle that forms between the first portion A₂a of the second sheath longitudinal axis A₂ and the device longitudinal axis A₁ when the exit port is aligned with the target. As FIG. 1 shows, the second portion A_{2b} of the second sheath longitudinal axis A₂ can be the portion of the second sheath longitudinal axis A₂ that that does not extend into the first sheath lumen 101_{L} after the second sheath 113 has been articulated from a non-deflected configuration (e.g., a straight configuration) to a deflected configuration (e.g., the curved configuration shown in FIG. 1). As another example, the second portion A_{2b} of the second sheath longitudinal axis A₂ can be the portion of the second sheath longitudinal axis A₂ that that remains in a non-deflected configuration (e.g., in a straight configuration) before and after the second sheath 113 is articulated from a non-deflected configuration (e.g., from a straight configuration) to a deflected configuration (e.g., to a curved configuration, such as to the curved configuration shown in FIG. 1).

For example, the angle 114 can be the angle between the portion of the device longitudinal axis A₁ that extends through the space 105 and the first portion A₂a of the second sheath longitudinal axis A₂. As another example, the angle 114 can be the angle between the first and second portions A₂ₐ, A_{2b} of the second sheath longitudinal axis A₂. The angle 114 can be, for example, about 90 degrees to about 140 degrees, including every 1 degree increment within this range (e.g., 90 degrees, 120 degrees, 140 degrees), to closely approximate (e.g., plus or minus 1 to 10 degrees) or match the entrance angle of the target location. The entrance angle of the target location can be measured against the same axis that the angle 114 is measured against, for example, against the device longitudinal axis A₁, or against the second portion A_{2b} of the second sheath longitudinal axis A₂. For example, FIG. 1 illustrates that the second sheath 113 can be articulated so that the angle 114 is 120 degrees (e.g., relative to the device longitudinal axis A₁) to match the entrance angle of the target location (e.g., the Ampulla of Vater 123), which FIG. 1 also shows as being 120 degrees (e.g., relative to the device longitudinal axis A₁). FIG. 1 illustrates that the first portion A₂a of the second sheath longitudinal axis A₂ can extend away from the device longitudinal axis A₁.

The angle 114 can be achieved by changing the shape of the second sheath 113 (e.g., a distal end of the second sheath 113) from a non-actuated configuration (also referred to as a non-deflected configuration) to an actuated configuration (also referred to as a deflected configuration). When the second sheath 113 is in the non-actuated configuration, the second sheath 113 can be curved, straight, or both. When the second sheath 113 is in the non-actuated configuration, the second sheath portion (e.g., the distal tip of the second sheath 113) that is deflectable into and out of the first sheath lumen 101_{L} and/or into and out of the space 105 can be outside of first sheath lumen 101, can be outside of the space 105, or can be outside of both. When the second sheath 113 is in an actuated configuration, the second sheath 113 can be curved. When the second sheath 113 is in an actuated configuration, the second sheath 113 can have one or multiple curves. For example, FIG. 1 illustrates that when the second sheath 113 is in an actuated configuration, the second sheath 113 can have a single curve. When the second sheath 113 is in an actuated configuration, the second sheath 113 can have a straight portion and a curved portion. For example, when the second sheath 113 is in an actuated configuration, the second sheath 113 can have the shape of a hook, whereby a distal portion of the second sheath 113 is curved and a proximal portion of the second sheath 113 is straight. The hook can be a closed hook or an open hook. For example, FIG. 1 illustrates that when the second sheath 113 is in an actuated configuration, the second sheath 113 can have the shape of an open hook, whereby a distal portion of the second sheath 113 is curved and a proximal portion of the second sheath 113 is straight. As FIG. 1 shows, when the second sheath 113 is in an actuated configuration (e.g., is in the shape of an open hook), the distal terminal end of the second sheath 113 can point away from the proximal portion of the second sheath 113, away from the device longitudinal axis A₁, or away from both as FIG. 1 shows. As FIG. 1 further shows, when the sheath 113 is in an actuated configuration (e.g., is in the shape of an open hook), the distal terminal end of the second sheath 113 can point away from the distal terminal end of the device 100 (e.g., away from the distal terminal end of the first sheath 101). When the second sheath 113 is in the actuated configuration, the distal terminal end of the second sheath 113 can point away from the distal terminal end of the device 100, toward the distal terminal end of the device 100, or perpendicularly toward the device longitudinal axis A₁.

FIG. 1 illustrates that the second sheath 113 can be actuated or moved by an actuator 115 (e.g., a wire, a rod), for example, from the non-actuated configuration (e.g., where the angle 114 is at 0 degrees) to any actuated position, where when the second sheath 113 is fully actuated by the actuator 115, the angle 114 can be 160 degrees. The actuator 115 (also referred to as the second sheath actuator) can be connected to a control, for example, at the device handle. By using the control, the operator of the device 100 can bend the distal end of the second sheath 113 to change the angle 114. For example, where the actuator 115 is a pull wire as shown in FIG. 1, applying tension to the pull wire via the control (e.g., by twisting or pushing a knob in a first direction) can cause the second sheath 113 to change from the non-actuated configuration to an actuated configuration, and releasing tension on the pull wire via the control (e.g., by twisting or pushing the knob in a second direction opposite the first direction) can cause the second sheath 113 to become less curved or return to the non-actuated configuration. The angle 114 can thereby be adjusted by actuating the actuator 115. FIG. 1 shows the second sheath 113 in a state of flexion, which can be useful in matching the angle of the common bile duct 124 or pancreatic duct 126 during cannulation. The markings 111 on the device 100 can include angle markings to indicate to the operator the angle 114 the second sheath 113 is at during use.

FIG. 1 illustrates that the radius of curvature of the bend in the distal end of the second sheath 113 (also referred to as the bend radius) can become progressively smaller as the angle 114 is increased, for example, from 90 degrees to 140 degrees, or more broadly, from 0 degrees to 160 degrees. For example, the bend radius of the second sheath 113 can decrease from about 15 mm to about 6 mm, including every 1 mm increment within this range (e.g., 15 mm, 12 mm, 6 mm), as the angle 114 increases from 90 degrees to 140 degrees, respectively, such that when the angle 114 is 120 degrees, the bend radius can be 12 mm.

When the second sheath 113 is at the desired angle 114 (e.g., 120 degrees) and the device 100 is aligned with the markings 111 as shown in FIG. 1, the operator can reliably know that device 100 is positioned in the lumen (e.g., the intestinal lumen 125) for cannulation of the target location (e.g., the Ampulla of Vater 123, the common bile duct 124, the pancreatic duct 126). FIG. 1 illustrates that when the second sheath 113, the third sheath 117, and the guidewire 119 are positioned in the space 105 and the second sheath 113 is at the angle 114, the second sheath 113, the third sheath 117, and the guidewire 119 can extend through the second and third openings 102b, 102c of the first sheath 101. As another example, FIG. 1 illustrates that the second sheath 113, the third sheath 117, and the guidewire 119 can be positioned in the space 105, extending through the second and third openings 102b, 102c of the first sheath 101, such that the second sheath 113 is at an angle 114 relative to the target location (e.g., Ampulla of Vater 123) and such that the exit port of the third sheath 117 via the second sheath 113 is aligned with the alignment markings 111. When the device 100 is aligned with the markings 111 as shown in FIG. 1, the operator can reliably know that device 100 is positioned in the lumen (e.g., the intestinal lumen 125) for cannulation of the target location (e.g., the Ampulla of Vater 123, the common bile duct 124, the pancreatic duct 126).

FIG. 2 illustrates that the device 100 can have two stabilizers 107, for example, a first stabilizer 107 and a second stabilizer 107.

FIG. 2 illustrates that the device 100 can have two actuators 115, for example, a second sheath first actuator 115 and a second sheath second actuator 115.

FIG. 2 illustrates that the stabilizers 107 can have a stabilizer width 107_{W}. The stabilizer width 107_{W} can be about 0.05 inches to about 0.25 inches wide, including every 0.01 inches within this range (e.g., 0.05 inches, 0.10 inches, 0.25 inches), but the stabilizer width 107_{W} may be sized to accommodate any anatomy, for example, altered anatomy, such that dimensions outside of this range are anticipated and dependent on the anatomy into which the device 100 is placed.

FIGS. 1 and 2 illustrate the distal port of the second sheath 113 in a fully raised (also referred to as fully elevated) position. The second sheath 113 can be in a fully raised position when the desired angle 114 is achieved by the operator, such that the fully raised position can correspond to any angle in the range of the angle 114 (e.g., 90 degrees to 140 degrees). FIGS. 1 and 2 illustrate that when the second sheath 113 is in the fully raised position, the distal port of the second sheath 113 can be in the space 105, and the second sheath lumen 113_{L} can be aligned with the target.

FIGS. 1 and 2 illustrate that the expander 109 can be distal to the second sheath 113 when the second sheath 113 is in an actuated configuration. As another example, the expander 109 can be proximal to the distal port of the second sheath 113 when the second sheath 113 is in an actuated configuration. In such a case, the expander 109 can be attached to or extend from the first sheath 101 on the opposite side of the third opening 102c than what is shown in FIGS. 1 and 2.

FIGS. 1 and 2 illustrate, for example, that the first sheath 101 can be an outer sheath and serve as the main structure of the device 100, that the endoscope 121 can be passed freely up to and/or beyond the space 105, that two stabilizers 107 can be adjacent to the space 105, that the endoscope 121 can be in a position proximal to the space 105, that the two stabilizers 107 can be expanded by the expander 109 for the purpose of stabilizing the device 100 and endoscope 121 in place within a lumen (e.g., within the duodenum), that the marking 111 on the stabilizers 107 can facilitate proper positioning of the device 100 relative to the to the target location (e.g., the Ampulla of Vater 123), that second sheath 113 can be actuated by the actuators 115, that the second sheath 113 can be articulated to form the angle 114 to match or closely approximate the entrance angle of the target location (e.g., the Ampulla of Vater 123) to facilitate cannulation, that the second sheath 113 can facilitate guiding the third sheath 117 during cannulation of the target location (e.g., the Ampulla of Vater 123), that the exit port of the third sheath 117 via the second sheath 113 can be aligned with the markings 111, and that the space 105 can permit full articulation of the endoscope 121 (e.g., forward-viewing endoscope) in direction 121a, or any combination thereof.

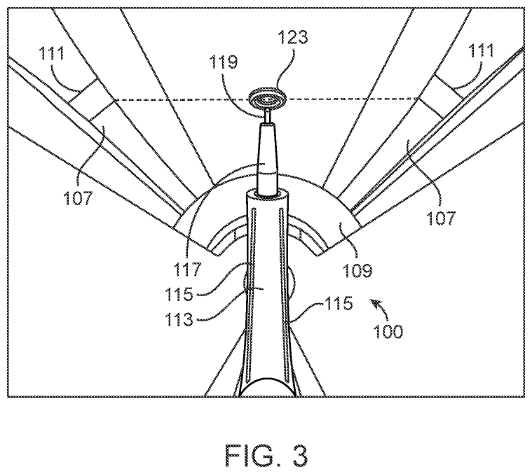

FIG. 3 illustrates the device 100 of FIGS. 1 and 2 from the endoscope perspective.

When the second and third sheaths 113, 117 are aligned relative to the alignment markings 111 as shown in FIG. 3, the second and third sheaths 113, 117 can be aligned with the target location. FIG. 3 can be a schematic representation of a digital image that the endoscope 121 can provide. The dashed line in FIG. 3 illustrates the relationship of the marks 111 to the target location (e.g., to the Ampulla of Vater 123). The dashed line may or may not be superimposed on the digital image to assist the user in aligning the second sheath 113, the third sheath 117, the guidewire 119, or any combination thereof with the target. As another example, the dashed line can be toggled on an off, for example, via an electronic control interface.

FIGS. 1-3 illustrate the device 100 in a fully expanded configuration. When the device 100 is in the fully expanded configuration, the engager 106 can be in a fully expanded configuration. The fully expanded configuration of the engager 106 may or may not correspond to the maximum expanded configuration that the engager 106 is capable of. In other words, the fully expanded configuration of the engager 106 can be dependent on the size of the lumen that the device 100 is in such that the fully expanded configuration can correspond to any expanded configuration of the engager 106 that results in the engager 106 securing the device 100 in the lumen. Thus, the fully expanded configuration of the engager 106 can also be referred to as a secured configuration, and any configuration in which the engager 106 does not secure the device 100 in the lumen can be referred to as an unsecured configuration. For example, FIGS. 1-3 illustrate that when the engager 106 is in the fully expanded configuration, the device 100 can be secured in the lumen (e.g., in the intestinal lumen 125). When the engager 106 is in a secured configuration in the lumen, the stabilizers 107 and/or the expanders 109 can inhibit or prevent longitudinal and/or rotational movement of the device 100 in the lumen. FIGS. 1-3 further illustrate that when the engager 106 is in a secured position in the lumen (e.g., when the stabilizers 107 and/or the expanders 109 contact tissue), the second sheath 113 can be deflected into alignment with the target (e.g., the Ampulla of Vater 123). For example, after the engager 106 is expanded to a secured configuration (e.g., the configuration shown in FIGS. 1-3), the second sheath 113 can be deflected into alignment with the target.

FIGS. 1-3 illustrate that the engager 106 can be expanded such that when the engager 106 is in a secured configuration, the target (e.g., the Ampulla of Vater 123) can be above the space 105, the target can be viewable through the space 105, the second sheath 113 can be alignable with the target, or any combination thereof. For example, FIGS. 1-3 illustrate that the engager 106 can be expanded such that the target (e.g., the Ampulla of Vater 123) is between two stabilizers (e.g., between the first and second stabilizers 107 in FIGS. 2 and 3) when the engager 106 is in a secured configuration. When the engager 106 is in a secured configuration, the target can be, for example, between an inner lateral edge of a first stabilizer 107 and an inner lateral edge of a second stabilizer 107.

When the engager 106 is in a secured configuration (e.g., the configuration shown in FIGS. 1-3), the target can advantageously be in a fixed position relative to the engager 106. When the engager 106 is in a secured configuration (e.g., the configuration shown in FIGS. 1-3), the target can advantageously be in a fixed position relative to the second sheath 113, the third sheath 117, the guidewire 119, or any combination thereof, for example, while the second sheath 113 is aligned with the target, while the third sheath 117 is advanced to the target, and/or while the guidewire 119 is advanced into the target. This can allow the second sheath 113, the third sheath 117, and/or the guidewire 119 to be moved relative to the target. As another example, the secured configuration can advantageously keep the target within the viewing window of the endoscope 121 during a procedure. The curved configuration of the second sheath 113 when the second sheath 113 is in an aligned configuration (e.g., the open hook shape in FIG. 1) can advantageously inhibit or prevent the second sheath 113, the third sheath 117, the guidewire 119, or any combination thereof from blocking or interfering with the view of the camera (e.g., endoscope 121) during the procedure.

When the engager 106 is in a secured configuration, the target can be inside the space 105 or outside of the space 105. For example, FIGS. 1-3 illustrate that when the engager 106 is in a secured configuration, the target can be outside of the space 105. When the target is outside of the space 105, the second sheath 113 can be deflected into an aligned configuration with the target, the target can be accessed (e.g., via the third sheath 117 and/or the guidewire 119), or both. As another example, the target can be in the space 105 when the engager 106 is in a secured configuration. The outward force of the engager 106 can, for example, force the target into the space 105, for example, by forcing the target to squeeze between the two stabilizers 107 shown in FIGS. 2 and 3 and into the space 105. When the target is inside of the space 105, the second sheath 113 can be deflected into an aligned configuration with the target, the target can be accessed (e.g., via the third sheath 117 and/or the guidewire 119), or both.

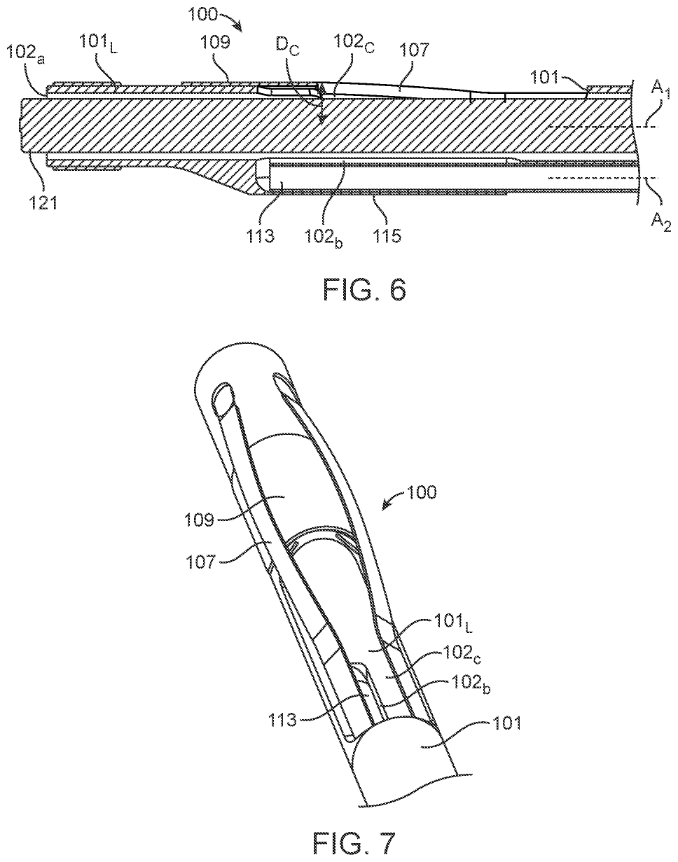

FIGS. 4 and 5 illustrate an exemplary unsecured configuration of the device 100 when the engager 106 is in a partially expanded configuration. A partially expanded configuration can be any expanded configuration that does not result in the engager 106 securing the device 100 in the lumen. For example, a partially expanded configuration can be any expanded configuration that is less than the fully expanded configuration of the engager 106 that results in the engager 106. For example, FIGS. 4 and 5 illustrate the engager 106 in a half-expanded configuration (e.g., halfway between the unexpanded configuration of FIGS. 6 and 7 and the fully expanded, secured, configuration of FIGS. 1-3), where the endoscope 121 is in a position proximal to the space 105, the expander 109 is in a half-expanded configuration (e.g., half-inflated), and the second sheath 113 is half-raised. The second sheath 113 can be in a half-raised position when the angle 114 is half of the desired angle 114, such that the half-raised position can correspond to any half-angle in the range of the desired angle 114 (e.g., 45 degrees to 70 degrees). When the engager 106 is in an unsecured configuration, longitudinal and/or rotational movement of the device 100 in the lumen may not be inhibited or prevented by the engager 106. FIGS. 4 and 5 illustrate that when the engager 106 is in an unsecured configuration, the second sheath 113 can be deflected. As another example, the second sheath may not be deflected until after the engager 106 is expanded to a secured configuration (e.g., the fully expanded configuration shown in FIGS. 1-3).

FIGS. 4 and 5 illustrate that the first sheath 101 can have a first sheath first lumen 101_{L1} and a first sheath second lumen 101_{L2}.

FIGS. 1-5 illustrate that the endoscope 121 can be proximal to the space 105 or in a proximal portion of the space 105 while the second sheath 113 is actuated to have the angle 114, during cannulation, or both. As another example, FIGS. 1-5 illustrate that the endoscope 121 can remain in the same position while the second sheath 113 is actuated to have the angle 114, during cannulation, or both.

FIGS. 6 and 7 illustrate the device 100 in an unexpanded configuration. When the device 100 is in the unexpanded configuration, the engager 106 can be in an unexpanded configuration. For example, FIGS. 6 and 7 illustrate the engager 106 in an unexpanded configuration, where the endoscope 121 is in a position distal to the space 105, the expander 109 is in an unexpanded configuration (e.g., fully deflated), and the second sheath 113 is in an undeployed (also referred to as non-actuated, non-deflected) configuration. When the expander 109 is in the unexpanded configuration, the stabilizers 107 can be in a non-expanded configuration. FIGS. 6 and 7 illustrate that when the stabilizers 107 are in the non-expanded configuration, the stabilizers 107 can lay flat. When the second sheath 113 is in the undeployed configuration, the second sheath 113 can lay beneath the first sheath lumen (e.g., 101_{L} or 101_{L1}) which can advantageously allow the endoscope 121 to advance through the distal end of the outer sheath 101, for example, past the first opening 102a. FIGS. 6 and 7 illustrate that the second sheath longitudinal axis A₂ can be offset and parallel to the device longitudinal axis A₁. FIG. 6 illustrates that when the engager 106 is in the unexpanded configuration, the contracted dimension Dc (also referred to as the unexpanded dimension) of the space 105 as measured from the peak of a stabilizer 107 to the device longitudinal axis A₁ can be about 5 mm to about 50 mm, including every 1 mm increment within this range (e.g., 5 mm, 25 mm, 50 mm). The peak of the stabilizer 107 when determining the contracted dimension Dc can be the point on the stabilizer 107 that is furthest from the device longitudinal axis A₁ when the engager 106 is in the unexpanded configuration. The contracted dimension Dc can be less than the expanded dimension D_{E}.

FIGS. 1-7 illustrate that the device 100 can be atraumatic and can slide easily over the mucosal surfaces, for example, from the mouth down to the duodenum (e.g., and beyond as needed).

FIGS. 1-7 illustrate that the device 100 can be expandable and contractible, for example, via the engager 106. For example, FIGS. 1-7 illustrate that the distal tip of the device 100 can be expandable and contractible via the engager 106.

FIGS. 1-7 illustrate that the device 100 can be selectively locked onto the endoscope 121, for example, onto the distal tip of the endoscope 121. The lock can be controlled by the operator, or the device 100 can be permanently locked onto the endoscope 121 for the duration of a procedure (e.g., an ERCP procedure). Locking the device 100 to the endoscope 121 can advantageously aid placement of the endoscope 121 and the device 100 relative to the target location (e.g., the Ampulla of Vater 123). For example, the first sheath 101 can be selectively locked to the endoscope 121. For example, the endoscope 121 and the first sheath 101 can be locked together via a cam lock. The cam lock can be moveable into and out of the first sheath lumen 101_{L}, for example, to engage and disengage with the endoscope 121. When the cam lock is engaged with the endoscope 121, the endoscope 121 can be temporarily locked in position. The endoscope 121 can be unlocked by disengaging the cam lock from the endoscope 121, for example, by moving the cam lock away from the endoscope 121 and out of the first sheath lumen 101L.

FIGS. 1-7 illustrate that the endoscope 121 can be a commercially available endoscope. As another example, cameras and/or light sources can be attached to, integrated with, or integrated into the body of the device 100 (e.g., the first sheath 101 and/or the second sheath 113) and images acquired by the camera can be transmitted via wired or wireless transmission to a viewing console (e.g., a computer screen such as a monitor or tablet). One or multiple data cameras can be incorporated with the device 100 to aid in visualization. For example, a camera can be attached to or integrated with the first sheath 101. As another example, a camera can be attached to or integrated with the second sheath 113. One or multiple illuminators (e.g., LED lighting, xenon lights) can be incorporated with the device 100 to aid in visualization. For example, an illuminator can be attached to or integrated with the first sheath 101. As another example, an illuminator can be attached to or integrated with the second sheath 113.

FIGS. 1-7 illustrate that one or multiple lumens can open into the space 105 or and/or can terminate at the distal end of the device 100 (e.g., at the distal terminal end of the device 100).

FIGS. 1-7 illustrate that one or multiple lumens can open into the space 105 or at the distal end of the device 100. When using the device 100, the operator can select which lumens to use. For example, when using the device 100, the operator can decide to use the exit of the lumen that extends from the space 105 to the distal end of the device 100 (e.g., the first sheath lumen 101_{L}) by advancing the endoscope 121 past the space 105. This can allow the operator to extend the endoscope 121 past the distal terminal end of the device 100 (e.g., past the distal terminal end of the first sheath 101), for example, during steering.

FIGS. 1-7 illustrate that the first sheath 101 can have, for example, one, two, three, four, five, six, or more accessory lumens (e.g., lumen 101_{L2}) also referred to as auxiliary lumens in addition to the main lumen (e.g., lumen 101_{L}). The main lumen can be the largest (e.g., largest diameter) lumen of the first sheath 101. The main lumen (e.g., lumen 101_{L}) can be the lumen through which the endoscope 121, the second sheath 113, the third sheath 117, the guidewire 119, or any combination thereof is positionable. Having multiple accessory lumens can be advantageous, as the accessory lumens can be dedicated to, for example, deployment of the guidewire 119, injection of contrast medium, inflation of the expanders 109, control of the actuators 115, communication with negative pressure sources, or any combination thereof. As another example, the second sheath 113, the third sheath 117, the guidewire 119, or any combination thereof can be moveable in an accessory lumen such that a distal tip of the second sheath 113 can be deflected from an accessory lumen (e.g., the lumen 101_{L2}) into the main lumen (e.g., the lumen 101_{L1}), and into the space 105. The distal tip of the second sheath 113 can be deflected back out of the space 105, back into the main lumen (e.g., the lumen 101_{L1}), and back into the accessory lumen (e.g., the lumen 101_{L2}).

FIGS. 1-7 illustrate that the guidewire 119 can be used at any point, for example, to aid cannulation or maintain intraductal stability.

FIGS. 1-7 illustrate that the device can have a single second sheath 113, and that the second sheath 113 can have a single working channel (e.g., lumen 113_{L}). As another example, the second sheath 113 can have multiple lumens, for example, 2 to 5 or more lumens, including every 1 lumen increment within this range. The outer diameter of the second channel lumen 113_{L} can be about 2 mm to about 6 mm, including every 1 mm increment within this range (e.g., 2 mm, 4 mm, 6 mm) and can actuate to at least 120 degrees from the device longitudinal axis A₁.

FIGS. 1-7 illustrate that the device 100 can have a moveable second sheath 113 having one or multiple second sheath lumens 113_{L}. For example, FIGS. 1-7 illustrate that the second sheath 113 can have the second sheath lumen 113_{L} (also referred to as the working channel 113_{L}). The working channel 113_{L} can have an unactuated position (e.g., FIGS. 6 & 7) and a fully actuated position (e.g., FIGS. 1-3). When the working channel 113_{L} is in the fully actuated position, the working channel 113_{L} can be aligned with the target. The working channel can have any partially actuated position between the unactuated position and the fully actuated position. For example, FIGS. 4 and 5 illustrate that the working channel can be in an actuated position halfway between the unactuated position and the fully actuated position. The fully actuated position of the working channel 113_{L} may or may not correspond to the maximum actuated position (e.g., maximum angle 114, maximum bend) that the working channel 113_{L} is capable of. In other words, the fully actuated position of the working channel 113_{L} can be dependent on the alignment (e.g., the angle 114) that is ultimately achieved with the target such that the fully actuated position can correspond to any actuated position of the working channel 113_{L} that results in the working channel 113_{L} being aligned with the target. Thus, the fully actuated position of the working channel 113_{L} can also be referred to as an aligned configuration, and any configuration in which the working channel 113_{L} is not aligned with the target can be referred to as an unaligned configuration.

When the working channel 113_{L} is in an unactuated position, the second sheath 113 can be in (e.g., rest in) a docking channel, for example, in the first sheath second lumen 101_{L2}. The first sheath second lumen 101_{L2} can be the docking channel. As another example, when the working channel 113_{L} is in an unactuated position, the second sheath 113 can be in a recess (also referred to as a docking bay) on the device 100. The recess can be, for example, a recess in the wall of the first sheath 101. The opening of the recess can be, for example, the second opening 102b. As another example, the recess can be an exposed portion of a distal portion of an accessory lumen, such as an exposed portion of the distal end of the first sheath second lumen 101_{L2}. When the second sheath 113 is in the recess and the second sheath 113 is in a non-actuated configuration (e.g., is straight as shown in FIGS. 6 and 7), the second sheath 113 may or may not extend into the main lumen (e.g., lumen 101_{L}, 101_{L1}) of the first sheath 101. For example, when the second sheath 113 is in the recess and the second sheath 113 is in a non-actuated position (e.g., is straight as shown in FIGS. 6 and 7), the second sheath 113 can be outside of the first sheath 101. As another example, FIGS. 1-7 illustrate that when the second sheath 113 is in the recess and is in a non-actuated configuration, the second sheath 113 may not extend into the main lumen of the first sheath 101.

FIGS. 1-7 illustrate that the recess can be a lumen, a port, or a groove. For example, the recess can be the distal end of the first sheath second lumen 101_{L2}. The lumens of the device 100 that are not the main lumen can be the auxiliary lumens (also referred to as accessory lumens) of the device 100. As another example, the recess can be a port or a groove in the wall of the first sheath 101 that defines the main lumen of the first sheath 101. As yet another example, the recess can be a port or a groove (e.g., of a tube) attached to the first sheath 101. The recess can advantageously allow the endoscope 121 to be advanced and retracted over the second sheath 113 when the device 100 is in an unexpanded configuration. For example, FIGS. 1-5 show the endoscope 121 proximal to and/or in the space 105 when the device is in various expanded configurations, FIG. 6 shows the endoscope 121 advanced partially out of the first sheath lumen 101_{L} while the device 100 is being steered, using the endoscope 121, to the target location, and FIG. 7 shows the endoscope 121 proximal to and/or in the space 105 when the device 100 is in an unexpanded configuration. FIG. 6 illustrates that when the device 100 is being steered to the target location, the device 100 can be in the unexpanded configuration. The arrangements shown in FIGS. 1-7 can advantageously remove the need for a side-facing camera, since the camera on the end of the endoscope 111 can view the Ampulla of Vater 112 through the space 105 when the endoscope 121 is in the retracted position shown in FIGS. 1-5. However, the device 100 can have a side-facing camera.

FIGS. 1-7 illustrate that manipulation of the actuators 115 can control the degree of actuation of the working channel. For example, FIGS. 1-7 illustrate that manipulation of the actuators 115 can control the angle 114 of the working channel 113_{L}. Manipulation of the actuators 115 can involve applying and releasing tension to the actuators 115. When the working channel 113_{L} is in an actuated position, the working channel 113_{L} may or may not extend beyond the engager 106 (e.g., beyond the stabilizers 107 and/or beyond the expanders 109). For example, FIGS. 1-5 illustrate that the working channel 113_{L} may not extend beyond the stabilizers 107 when the second sheath 113 is in a fully actuated position. As another example, the working channel 113_{L} can extend beyond the stabilizers 107 when the second sheath 113 is in a fully actuated position.

During deployment of the device 100 to the target location (e.g., the Ampulla of Vater 123), the device 100 can have the unexpanded configuration shown in FIG. 6 or FIG. 7. Once the target location is identified and the device 100 is in the desired position, the endoscope 121 can be retracted to the position shown in FIG. 1-5. Before or after the endoscope 121 is retracted to this position (also referred to as the cannulation viewing position), the expander 109 can be expanded to extend the stabilizers 107 away from the device longitudinal axis A₁ to stabilize the position of the device 100 at the target location. Once the device 100 is stabilized, the working channel 113_{L} can be moved to the aligned configuration (e.g., also referred to as the cannulation position) shown in FIGS. 1-3, for example, by moving the second sheath 113 out of the docking channel (e.g., the first sheath second lumen 101_{L2}) and into the space 105. The working channel 113_{L} can be positioned and angled to present the third sheath 117 at the exact or approximate angle that the target location (e.g., the Ampulla of Vater 123) is in, after which the guidewire 119 can be advanced into the Ampulla of Vater 123 through the third sheath 117. Once the guidewire 119 is in position, the third sheath 117 can be retracted.

FIGS. 1-7 illustrate that the device 100 can advantageously optimize (e.g., minimize) the distance between the end of the working channel 113_{L} and the target location and reduce or eliminate the need to adjust the device 100 during cannulation since the second sheath 113 can be adjustable once the device 100 is in position and stabilized via the engager 106 (e.g., via the stabilizers 107 and/or the expanders 109). For example, the angle 114 can be adjustable and the radius of curvature of the bend of the second sheath 113 when the second sheath 113 is in a deflected configuration can be adjustable.

FIGS. 1-7 illustrate that the endoscope 121 can be moveable into and out of the space 105.

FIGS. 1-7 illustrate that the second sheath 113 can extend along the entire length of the first sheath 101 or a portion thereof (e.g., only along the distal portion of the first sheath 101).

FIGS. 1-7 illustrate that the second sheath 113 can be moveable relative to the first sheath 101. The second sheath 113 can be independently moveable relative to the first sheath 101. For example, the second sheath 113 can be deflectable into and out of the space 105 to align the second sheath 113 with the target while the first sheath 101 is secured in position in the lumen (e.g., the intestinal lumen 125) via the engager 106.

FIGS. 1-7 illustrate that the third sheath 117 can be moveable relative to the first and second sheaths 101, 113. The third sheath 117 can be independently moveable relative to the second sheath 113. For example, when the second sheath 113 is in an aligned configuration, the third sheath 117 can be translated out of the second sheath 113 toward and/or into the target. The second sheath 113 can be kept in the aligned configuration while the third sheath 117 is advanced out of and retracted into the second sheath 113.

FIGS. 1-7 illustrate that the guidewire 119 can be moveable relative to the first, second, and third sheaths 101, 113, 117. The guidewire 119 can be independently moveable relative to the second and third sheaths 113, 117. For example, when the second sheath 113 is in an aligned configuration and the third sheath 117 has cannulated the target, the guidewire 119 can be translated out of the third sheath 117 into the target. The second and third sheaths 113, 117 can be kept in the aligned configuration while the guidewire 119 is advanced out of the second sheath 113.

FIGS. 1-7 illustrate, for example, that the second sheath 113 can be longitudinally translatable, for example, in directions 121a and 121b. The second sheath 113 can be translated in directions 121a and 121b relative to the first sheath 101 when the second sheath 113 is in a non-actuated position and/or when the second sheath 113 is in an actuated position. For example, when the second sheath 113 is in the actuated configuration shown in FIG. 1, the second sheath 113 can be moved in directions 121a and 121b back and forth in the space 105. The second sheath 113 can maintain the actuated configuration while the second sheath 113 is being moved in directions 121a and 121b. As another example, FIGS. 1-7 illustrate that the second sheath 113 may not be longitudinally translatable. In either case (e.g., longitudinally translatable or not longitudinally translatable), the second sheath 113 can be articulated into and out of the recess (e.g., into and out of the first sheath second lumen 101_{L2}), for example, via the actuators 115. The actuators 115 can be articulated individually or together. For example, FIGS. 1-7 illustrate that the device can have a first actuator 115 and a second actuator 115, whereby actuating both of the actuators 115 together (e.g., pulling and pulling) can move the second sheath 113 out of the recess toward the target location and into the space 105 adjacent the target location, for example, in a first plane having the device longitudinal axis A1, where the first plane can be the cross-sectional plane shown in FIG. 1. By pulling only one of the actuators 115 at a time, the position of the distal tip of the second sheath 113 can be adjusted along an axis or a second plane perpendicular to both the first plane and the device longitudinal axis A₁ (e.g., into and out of the sheet having FIG. 2), so that the distal tip of the second sheath 113 can be moved laterally (e.g., left and right by pulling one or the other actuators 115) in addition to radially (e.g., up and down by pulling and releasing both of the actuators 115 at the same time) relative to the device longitudinal axis A₁. As another example, the device 100 can have a single actuator 115 (e.g., only one wire) that tension can be applied to and released from to deflect the second sheath 113 into and out of the space 105 to align the second sheath 113 with the target.

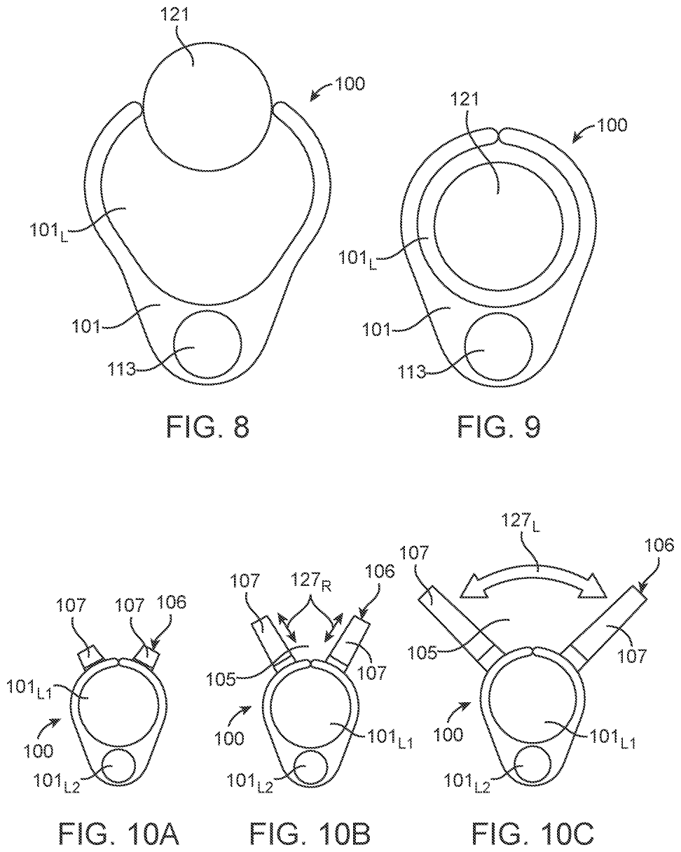

FIGS. 8 and 9 illustrate that the first sheath 101 can be split to allow installation over the shaft of the endoscope 121, where FIG. 8 shows the split sheath 101 in an open configuration and FIG. 9 shows the split sheath 101 in a closed configuration. FIG. 9 illustrates the endoscope 121 installed in the first sheath 101.

FIGS. 10A-10C illustrate that the stabilizers 107 can move radially in the directions indicated by the double-headed arrow 127R, can move laterally in the directions indicated by the double-headed arrow 127_{L}, or any combination thereof, in order to manipulate tissue (e.g., tissue 125_{T}). For example, FIG. 10A illustrates the stabilizers 107 in an unexpanded configuration, FIG. 10B illustrates the stabilizers 107 of FIG. 10A in an expanded configuration having been moved radially outward along arrow 127_{R}, and FIG. 10C illustrates the stabilizers 107 of FIG. 10A in an expanded configuration having been moved radially and laterally outward along arrows 127_{R} and 127_{L}. As another example, the stabilizers 107 of FIGS. 10B and 10C can be moved radially and/or laterally inward along arrows 127_{R} and/or 127_{L}, for example, to return the stabilizers 107 to the unexpanded configuration shown in FIG. 10A. The radial movement along arrow 127_{R} can be a primary movement direction. The lateral movement along arrow 127_{L} can be a secondary movement direction. The laterally outward positions of the stabilizers 107 in FIG. 10C, for example, illustrate an exemplary splay feature state, in which the stabilizers 107 have been splayed laterally outward. The stabilizers 107 can be moved radially and then laterally, laterally and then radially, or radially and laterally at the same time.

The stabilizers 107 and the expander 109 can be separate from one another such that the stabilizers 107 do not contact, are not attached to, or are not integrated with the expander 109. In such a case, the stabilizers 107 and the expander 109 can be expanded and contracted independently of each other such that expansion or contraction of the stabilizers 107 does not affect the expansion or contraction of the expander 109 and vice versa. As another example, the expansion and contraction of the expander 109 can cause the stabilizers 107 to expand and contract, respectively. For example, the expander 109 can be in contact with, can be attached to, or can be integrated with one or multiple stabilizers 107 (e.g., the first and second stabilizers 107 shown in FIGS. 1 and 2 and in FIGS. 10A-10C). In such configurations, the expander 109 can extend or expand the stabilizers 107 outward (e.g., radially outward along arrow 127_{R}) away from the device longitudinal axis A₁, laterally outward (e.g., laterally outward along arrow 127_{L}), or along both arrows 127_{R} and 127_{L} such that expansion of the expander 109 causes outward extension or expansion of one, some, or all of the stabilizers 107 (e.g., of both of the stabilizers 107 in FIGS. 1 and 2). For example, FIGS. 1-7 illustrate that the articulation (e.g., expansion, inflation, extension, bending) of the stabilizers 107 can be driven by or be caused by the articulation (e.g., expansion, inflation, extension, bending) of the expander 109. When the expander 109 is expanded, the expander 109 can push and/or pull the stabilizers 107 outward. For example, FIGS. 1-7 illustrate that when the expander 109 is expanded, the expander 109 can push the stabilizers 107 outward to the positions shown in FIGS. 1 and 2. As another example, the stabilizers 107 and the expander 109 can be articulated (e.g., expanded, inflated, extended, bent) independently from each other such that the stabilizers 107 can be articulated (e.g., expanded, inflated, extended, bent) away from the device longitudinal axis A₁ with or without simultaneous articulation (e.g., expansion, inflation, extension, bending) of the expander 109, and vice versa.

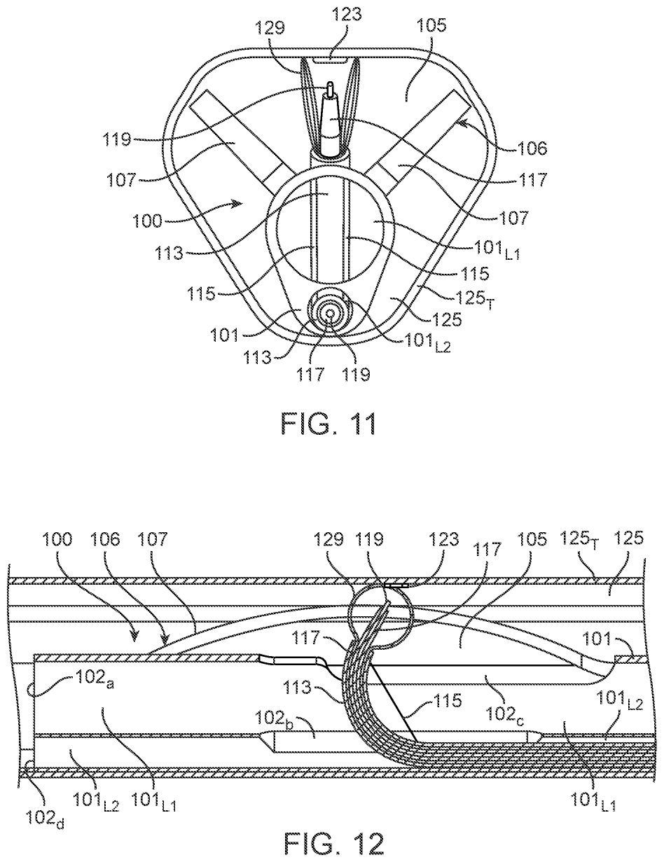

FIGS. 11 and 12 illustrate that the distal tip of the second sheath 113 can have a tissue manipulator 129 that can be used to manipulate tissue (e.g., tissue 125_{T}) and to provide space for visualization during cannulation of the target location (e.g., the Ampulla of Vater 123). The tissue manipulator 129 can allow separation of the mucosal surface (e.g., the tissue 125_{T}) from the distal tip of the second sheath 113 and allow the operator to visualize the exit of the third sheath 117 from the second sheath 113. The tissue manipulator 129 can be, for example, one or multiple wires (e.g., a wire tulip) or an inflatable balloon. For example, FIGS. 11 and 12 illustrate that the tissue manipulator 129 can be a wire tulip (also referred to as a tissue manipulator wire). The tissue manipulator 129 can have a fixed state or can be selectively extended, retracted, contracted, and/or expanded by the operator. For example, the tissue manipulator can be a fixed cage, an extendible cage, or an expandable and contractible cage. The tissue manipulator 129 can be, for example, extended from a non-extended configuration to an extended configuration. As another example, the tissue manipulator 129 can be expanded from an unexpanded configuration to an expanded configuration. For example, the tissue manipulator 129 can be attached to the distal end of the second sheath 113. When the distal tip of the second sheath 113 is in a non-deflected configuration (e.g., in a recess in the first sheath 101), the tissue manipulator 129 can be in a contracted configuration in the recess with the second sheath 113. The tissue manipulator 129 can be biased to expand into an expanded configuration (e.g., the expanded configuration shown in FIGS. 11 and 12) when the distal tip of the second sheath 113 is deflected into the space to align the second sheath 113 with the target. Thus, movement of the second sheath 113 out of the recess can cause the tissue manipulator 129 to expand into an expanded configuration. Movement of the second sheath 113 and the tissue manipulator 129 back into the recess can cause the tissue manipulator 129 to collapse back into a contracted configuration. The tissue manipulator 129 can be locked into place. As another example, the tissue manipulator may not be locked into place. Extension and/or angulation of the tissue manipulator 129 can be mechanically or electronically actuated. FIG. 12 further illustrates that the first sheath second lumen 101_{L2} can form a fourth opening 102d such that the first opening 102a of the first sheath 101 and the fourth opening 102d of the first sheath 102d form distal openings of the device 100.

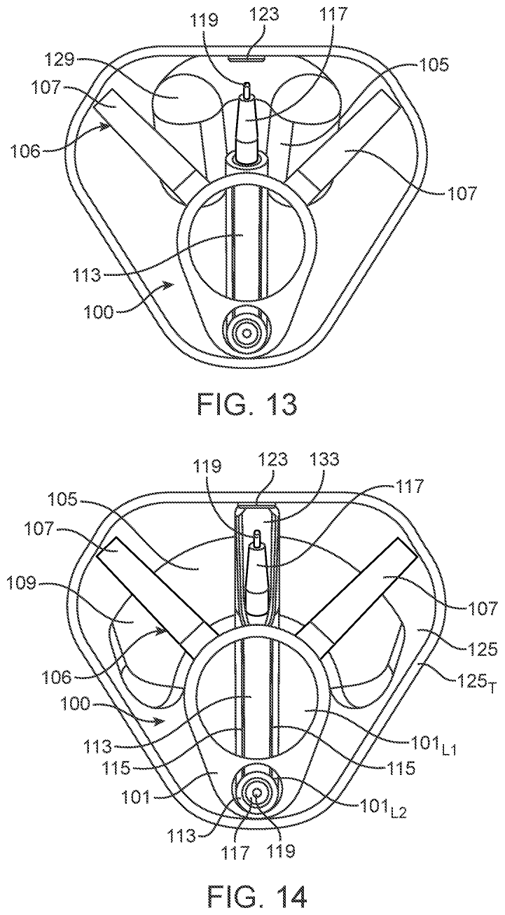

FIG. 13 illustrates that the tissue manipulator 129 can be a balloon (also referred to as a tissue manipulator balloon). The tissue manipulator balloon can be attached to the second sheath 113 and can be separate from the expander 109. As another example, the tissue manipulator balloon can be separate from the second sheath 113 and can be attached to the expander 109.

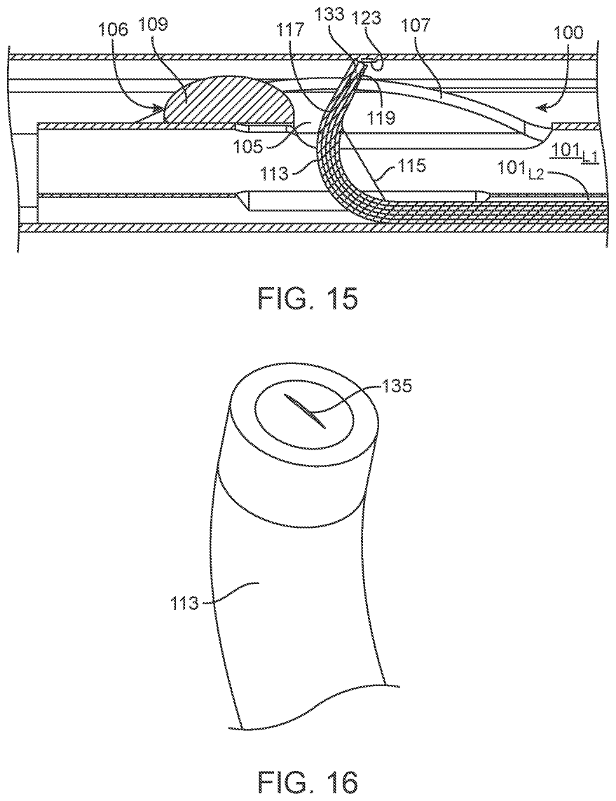

FIGS. 14 and 15 illustrate that the second sheath 113 can have a beveled distal tip 133 for tissue manipulation and to provide space for visualization during cannulation of the target location (e.g., the Ampulla of Vater 123). The tip of the second sheath lumen 113_{L} can be beveled to more easily visualize the exit of tools (e.g., the third sheath 113, the guidewire 119) from the second sheath lumen 113_{L} by the operator. FIGS. 14 and 15 illustrate the second sheath 113 in an extended configuration. FIGS. 14 and 15 illustrate that when the second sheath 113 is in an actuated configuration (e.g., an aligned configuration), the second sheath 113 can extend out of the space 105, for example, beyond the engager 106 (e.g., beyond stabilizers 107).

FIG. 16 illustrates that the distal tip of the second sheath 113 can have a one-way check valve 135 to inhibit or prevent reflux of body or scope fluids into the second sheath lumen 113_{L} (also referred to as the working channel). The one-way valve 135 can, for example, inhibit or prevent fluids entering the working channel 113_{L} in a retrograde fashion.

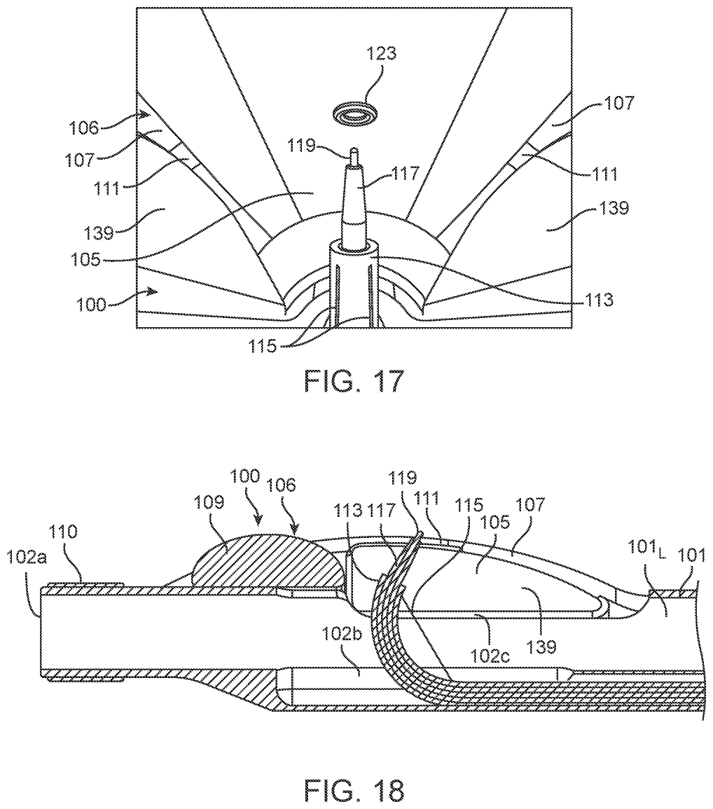

FIGS. 17 and 18 illustrates that the stabilizers 107 can be skirted by balloons 139 (also referred to as skirt balloons) to inhibit or prevent tissue (e.g., tissue 125_{T}) from blocking the view of the endoscope 121. The balloons 139 can be lateral of the stabilizers 107. The expander 109 can be distal of the balloons 139. The balloons 139 can assist in expanding the stabilizers 107. The balloons 139 may not assist in expanding the stabilizers 107. When the balloons 139 are expanded, the balloons 139 can help stabilize the device 100 in the lumen. The balloons 139 can be expanded independently of the expander 109. As another example, the expanders 109 and the balloons 139 can be inflated together. For example, the same inflation lumen can be connected to the expanders 109 and the balloons 139.

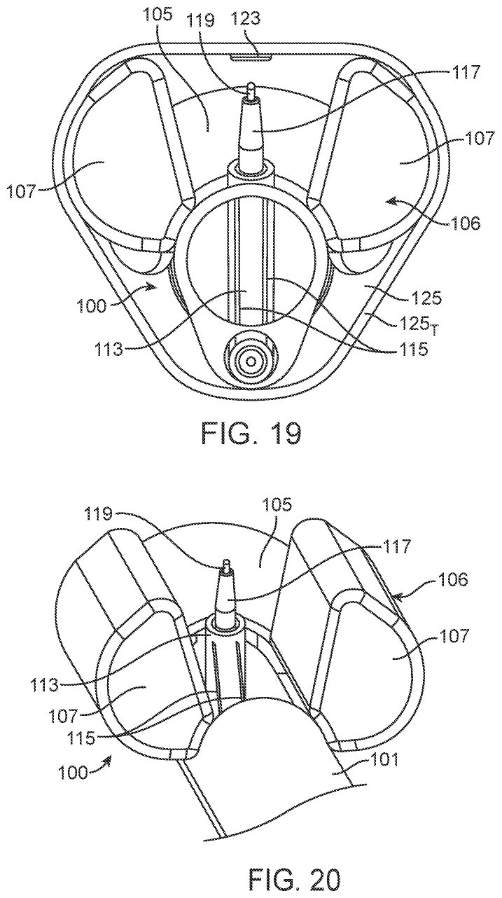

FIGS. 19 and 20 illustrate a variation of the device 100 in which the stabilizers 107 can be balloons (also referred to as stabilizer balloons). The stabilizer balloons can be inflatable and deflatable. When the stabilizers 107 are balloons, the device 100 may or may not have the expander 109.

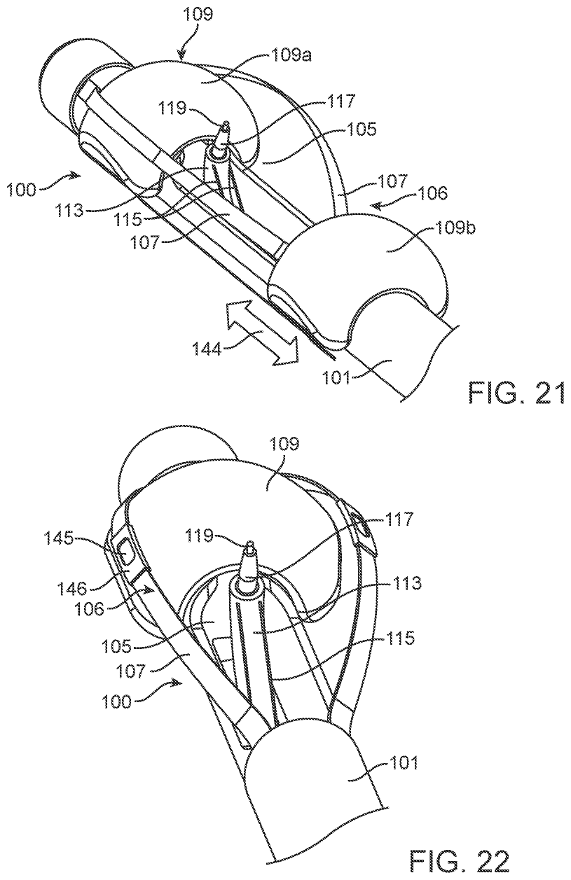

FIG. 21 illustrates that the device 100 can have multiple expanders 109, for example, a first expander 109a and a second expander 109b. The first and second expanders 109a, 109b can both be balloons. The device can have the second expander 109b, for example, in addition to the first expander 109a, for additional stabilization and tissue manipulation. The first and second expanders 109a, 109b can have fixed positions. As another example, the first balloon 109a and/or the second balloon 109b can be longitudinally movable. The first balloon 109 and/or the second balloon 109b can be longitudinally moveable when unexpanded and/or when expanded. For example, FIG. 21 illustrates that the secondary balloon 143 can be moved longitudinally in the directions indicated by the double-headed arrow 144, for example, along the outside of the first sheath 101, toward and away from a distal terminal end of the device 100. The directions indicated by the arrow 144 can be parallel to the device longitudinal axis A₁. As yet another example, the device 100 can have the first and second expanders 109a, 109b without any stabilizers 107. In such cases, the first and second expanders 109a, 109b can function as stabilizers.

FIG. 22 illustrates that the stabilizers 107 can have suction ports 145 and/or a gripping texture 146 for additional stabilization and tissue manipulation.

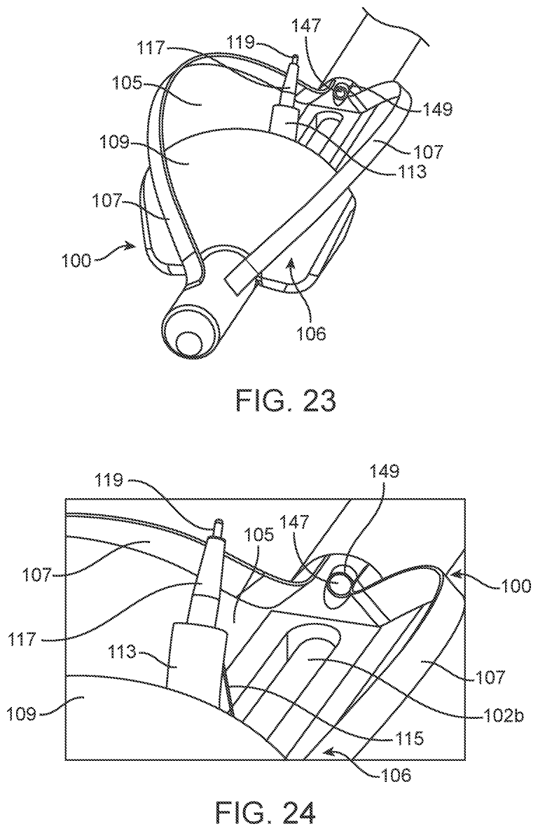

FIGS. 23-25 illustrate that the device 100 can have an integrated camera 147 and an integrated light source 149 (also referred to as illuminator) to eliminate the need for a separate endoscope (e.g., to eliminate the need for the endoscope 121). The integrated camera and light source 147, 149 can be fixed or moveable, for example, longitudinally moveable like the endoscope 121. FIGS. 23-25 illustrate that the camera and light source 147, 149 can be attached to the first sheath 101. As another example, the camera and light source 147, 149 can be attached to the second sheath 113 (e.g., to the distal end of the second sheath 113).

FIG. 26 illustrates the device 100 can have one or multiple stabilizer holders 60, for example, one stabilizer holder 60 for each of the stabilizers 107. The stabilizer holder 60 can secure the stabilizers 107 to the extender 109. FIG. 26 illustrates that the stabilizer holder 60 have a stabilizer channel through which the stabilizers 107 extend. For example, the stabilizer holder 60 can be a sleeve or ring having a channel through which a stabilizer 107 can be in. As another example, the stabilizer holder 60 can be attached to or integrated with the extender 109 such that the stabilizer holder 60 forms a loop through which the stabilizers 107 can extend, for example, like a belt extends through a belt loop. As yet another example, the portion of the expander 109 that is in contact with (e.g., beneath) the stabilizers 107 can have a stabilizer channel (e.g., one for each stabilizer 107) that the stabilizers 107 can lay against. The stabilizer channel can be, for example, a groove in the expander surface. The stabilizer channel can be, for example, a groove in the surface of the expander 109. The stabilizer holder 60 can control the radial and lateral movement (e.g., in directions 127_{R} and 127_{L}) of the stabilizers 107. For example, the stabilizer holder 60 can inhibit or prevent lateral movement (e.g., in directions 127_{L}) of the stabilizers 107 but allow radial movement (in directions 127_{R}) of the stabilizers 107. As another example, the stabilizer holder 60 can allow both lateral and radial movement (e.g., in directions 127_{R} and 127_{L}) of the stabilizers 107 such that the expander 109 can, via the stabilizer holder 60, be used to control the amount of lateral movement (e.g., in directions 127_{L}) of the stabilizers 107 when the expander 109 is expanded, thereby allowing the expander 109 to control the extent that the stabilizers 107 splay laterally outward away from each other when the expander 109 is expanded. For example, the placement, angle, and/or channel depth of the stabilizer holder 60 in the expander 109 can be selected to achieve the amount of lateral movement desired.

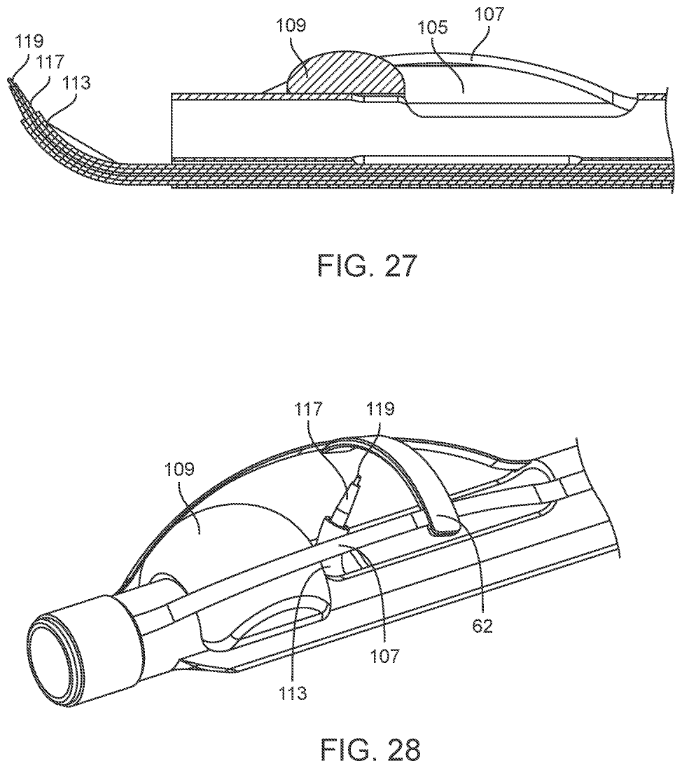

FIG. 27 illustrates that the second sheath 113, the third sheath 117, and the guidewire 119 can be translated out of a distal terminal port of the first sheath 101 (e.g., out of the second lumen 101_{L2}).

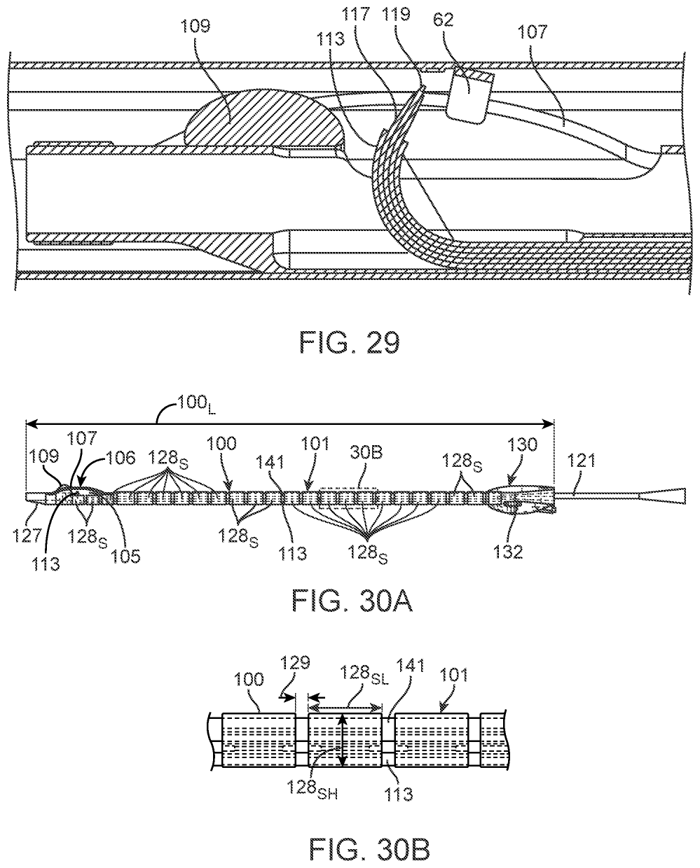

FIGS. 28 and 29 illustrate that the device 100 can have a bridge 62 between two stabilizers 107, between a stabilizer 107 and the expander 109 (e.g., between one of the stabilizers 107 in FIGS. 1-7 and the expander 109, between the other of the stabilizers 107 in FIGS. 1-7 and the expander 109, and/or between the two stabilizers 107, for example, as shown in FIGS. 28 and 29), or both. For example, FIGS. 28 and 29 illustrate that the bridge 62 can be attached to first and a second stabilizers 107 of the device 100. The bridge 62 can extend between the first and second stabilizers 107. The bridge 62 can be, for example, perpendicular to the device longitudinal axis A₁. The bridge 62 can help smooth out the mucosa (e.g., tissue 125_{T}) when the stabilizers 107 and/or the expander 109 are in an expanded or extended configuration. The bridge 62 can be a non-elastic bridge or an elastomeric bridge. For example, the bridge 62 can be a sheet of elastic material without an inner cavity (e.g., not a balloon). As another example, the bridge 62 can be an elastic vessel having an expandable cavity (e.g., a balloon). As yet another example, the bridge 62 can be a rod. For example, FIGS. 28 and 29 illustrate that the bridge 62 can be a rod. The rod can be, for example, a strut that connects the first and second stabilizers 107 in FIGS. 28 and 29 together. The rod can be flexible or inflexible. The bridge 62 can be straight or curved. For example, FIG. 28 illustrates that het bridge 62 can be curved.

The bridge 62 can increase in length from a bridge first length to a bridge second length when the engager 106 (e.g., stabilizers 107 and/or expanders 109) is expanded to accommodate an increased distance between the stabilizers 107 as the stabilizers are moved from an unexpanded configuration to an expanded configuration, for example, by stretching or by having a bridge first rod that is moveable relative to a bridge second rod such as along tracks (e.g., tongue and grooves) or such as a telescopable arrangement (e.g., half of the rod is telescopable into and out of a second half of the rod). The bridge 62 can decrease from the bridge second length to the bridge first length when the engager 106 is contracted. As another example, the bridge 62 can be rigid, for example, to keep the stabilizers 107 a constant distance from each other as the engager 106 is expanded and contracted. The bridge can be attached to or integrated with the one or multiple stabilizers 107 (e.g., the stabilizers in FIG. 1-7), with one or multiple expanders 109 (e.g., the expander 109 in FIG. 1-7), or with one or multiple stabilizers 107 and with one or multiple expanders 109. The bridge 62 can be separate from the engager 106 or can be part of the engager 106 such that the bridge 62 may or may not expand or assist with the expansion of the device 100. As still yet another example, the bridge 62 can be a portion of the expander 109. The bridge 62 can be, for example, the portion of the expander 109 (e.g., a portion of a balloon) that extends between the two stabilizers 107 shown in FIG. 2. In such cases, the bridge 62 can be expandable such that expansion of the bridge 62 causes expansion of the device 100. The bridge 62 can help smooth out the mucosa when the engager 106 is in an expanded or extended configuration.

FIGS. 28 and 29 illustrate that the bridge 62 can be separate from the expander 109 such that the bridge 62 can be attached to the stabilizers 107 and extend across the space 105 when the device 100 is in the expanded configuration. For example, the device 100 can have two stabilizers 107 (e.g., as shown in FIGS. 1, 2, 28, and 29), an expander 109 (e.g., as shown in FIGS. 1 and 2), and a bridge (e.g., not shown in FIGS. 1, 2, 28, and 29). The bridge 62 can be transparent so that the bridge can smooth out the mucosa without impeding visualization of the target location. As another example, the device 100 may not have the bridge 62 (e.g., as shown in FIGS. 1 and 2), whereby the expander 109 and/or the stabilizers 107 can smooth out the mucosa. The stabilizers 107 and/or the expander 109 (e.g., a balloon) can be transparent such that that tissue can be viewed through the expander 109 when the expander 109 is in the unexpanded configuration and/or when the expander 109 is in an expanded configuration. As another example, the stabilizers 107 and/or the expander 109 (e.g., a balloon) can be opaque to focus the operator's attention (e.g., by blocking view of tissue that the stabilizers 107 and/or the expander 109 are in front of) on the target location being viewed through the space 105 via the endoscope 121.

The bridge 62 can be slideable along the stabilizers 107 such that the position of the bridge 62 relative to the device longitudinal axis A₁ can be adjusted forward and backward (e.g., in directions 121a and 121b), for example, via a control on or in the handle of the device 100 via a wire or wires attached to the bridge 62 and to the control. As another example, the bridge 62 can be fixed to the stabilizers 107 such that the longitudinal position relative to the device longitudinal axis A₁ cannot be adjusted. The bridge 62 can be in a fixed position anywhere along the length of the stabilizers 107, for example, in the position shown in FIGS. 28 and 29.

FIGS. 1-3, 11-15, and 17-29 illustrate the device 100 in a fully expanded configuration with the second sheath at the desired angle 114. As another example, FIGS. 11-15 and 17-25 illustrate the device 100 in a partially expanded configuration with the second sheath at the desired angle 114.

FIGS. 1-29 illustrate that the endoscope 121 can slide longitudinally back and forth within the device during the procedures.

FIGS. 1-29 illustrate that the device 100 can (1) provide stability for the endoscope 121 and endoscopic tools, for example, during ERCP procedures, (2) improve visualization of the target location (e.g., the Ampulla of Vater 123), (3) provide an accessory channel to facilitate cannulation, or any combination thereof. For example, FIGS. 1-29 illustrate that the device 100 can facilitate easy cannulation of the target location (e.g., Ampulla of Vater 123) through the combination of (1) stabilizing the working channel 113_{L} within the tissue lumen (e.g., intestinal lumen 125) relative to the target location, (2) improving the visualization of the target location (3) the working channel 113_{L} being alignable with the target location to permit simple cannulation using the endoscope 121, and (4) a sterile channel with anti-microbial features (e.g., having the one-way valve 135), or any combination thereof.

FIG. 30A illustrates that the first sheath 101 can be flexible and/or segmented so that the device 100 can bend as the device is advanced to and withdrawn from the target (e.g., the Ampulla of Vater 123). For example, FIG. 30A illustrates that the first sheath 101 can have sheath segments 128_{S}. The sheath segments 128_{S} can be rigid. The sheath segments 128_{S} can be flexible. The space 129 between every two sheath segments 128_{S} can be a pivot point at which the first sheath 101 can bend. The sheath segments 128_{S} and the spaces 129 between the sheath segments 128_{S} can allow the device 100 to bend as the device 100 is advanced to and withdrawn from the target. As another example, the first sheath 101 can be a flexible, non-segmented sheath. The first sheath 101 (e.g., segmented or not segmented) can navigate the bends necessary to reach the target by bending or flexing. For example, the anatomy that needs to be traversed to reach the Ampulla of Vater 123 has a number of bends in it and those bends have variable geometry. To reach the Ampulla of Vater 123, the flexibility and/or the sheath segments 128_{S} of the first sheath 101 can allow the device 100 to be navigated through such areas as the pharynx and the path going from the esophagus into the stomach, around, and into the duodenum.

FIG. 30A illustrates that the sheath segments 128_{S} can have a uniform length and height. For example, the sheath segments 128_{S} can have a uniform length and width until the distal tip is reached, at which point the length of the sheath segments 128_{S} can be adjusted or varied to accommodate the features at the distal tip of the device 100 (e.g., the engager 106, the first, second, and third openings 102a, 102b, 102c). As another example, the sheath segments 128_{S} can get progressively shorter from the proximal end of the first sheath 101 to the distal end of the first sheath 101 to allow for progressively greater deflection closer to the distal tip of the device 100.

FIG. 30A illustrates that the device 100 can have a fourth sheath 141. For example, the device 100 can have the first sheath 101, the second sheath 113, the third sheath 117, the fourth sheath 141, or any combination thereof. The second and fourth sheaths 113, 141 can extend through the first sheath 101. For example, the fourth sheath 141 can extend through the first sheath first lumen 101, and the second sheath 113 can extend through the first sheath second lumen 101_{L2}. FIG. 30A illustrates that the second and fourth sheaths 113, 141 can extend through lumens in the sheath segments 128_{S}. For example, each sheath segment 128_{S} can have a first sheath first lumen 101_{L1} and a first sheath second lumen 101_{L2}. The outer surface of the second sheath 113 and/or the fourth sheath 141 can be attached to the inner surface of the first sheath 101, for example, to the inner surface of a non-segmented first sheath 101, or to the inner surfaces of the sheath segments 128_{S}. The second and fourth sheaths 113, 141 can be attached to the first sheath 101, for example, with adhesive such as glue. As another example, the second sheath 113 and/or the fourth sheath 141 can float in the lumens in the first sheath 101, for example, in the first sheath first and second lumens 101, 101_{L2}, or in lumens in the sheath segments 128_{S}. As another example, the second sheath 113 can be translatable in the first sheath 101 and/or the fourth sheath 141 can be translatable in the first sheath 101, for example, in the sheath segments 128_{S}. The third sheath 117 and the guidewire 119 can be moveable (e.g., advanceable and retractable) in the second sheath 113.

FIG. 30A illustrates that the device 100 can have a handle 130. The proximal end of the first sheath 101 can be attached to the handle 130. The first sheath 101 can extend from the handle 130 to the distal tip of the device 100. The handle 130 can be transparent or opaque. For example FIG. 30A illustrates that the handle 130 can be transparent.

FIG. 30A illustrates that the first sheath 101 and the second sheath 113 can extend from the handle 130 to the distal tip of the device 100. The first and second sheaths 101, 113 can extend from a proximal end of the device 100 to a distal end of the device 100 through lumens in the first sheath 101, for example, through lumens in the sheath segments 128_{S}. The first and second sheaths 101, 113 can be connected to the handle 130. The first and second sheaths 101, 113 can extend into the handle 130. The first and second sheaths 101, 113 can be attached to the first sheath 101, for example, via adhesive such as glue. An outer surface of the first and second sheaths 101, 113 can be attached to the first sheath 101, for example, at each sheath segment 128_{S}. As another example, the first sheath 101 and/or the second sheath 113 can be longitudinally translatable (e.g., in directions 121a and 121b) in the first sheath 101.

FIG. 30A illustrates that a proximal end of the stabilizers 107 can be attached to one of the sheath segments 128_{S} and that the distal end of the stabilizers 107 can be attached to another one of the sheath segments 128_{S}.

FIG. 30A illustrates that the handle 130 can have a control 132 (also referred to as the second sheath control 132) to control the configuration of the second sheath 113. The control 132 can be used to move (e.g., raise and lower) the distal terminal end of the second sheath 113 into and out of the space 105 to align the second sheath 113 with the target. The control can be a moveable control such as a knob, a wheel, or a slider. For example, FIG. 30A illustrates that the control 132 can be a rotatable wheel on a side of the handle 130. Rotating the control 132 in a first direction (e.g., clockwise direction) can move the distal tip of the second sheath 113 toward the engager 106 (e.g., and toward the target), and rotating the control 132 in a second direction (e.g., counterclockwise direction) can move the distal tip of the second sheath 113 away from the engager 106 (e.g., and away from the target), or vice versa. For example, rotating the control 132 in the first direction (e.g., clockwise direction) can move the distal tip of the second sheath 113 to the position shown in FIG. 30A. The control 132 can have a ratchet so that as the second sheath 113 is moved into the space 105, the second sheath 113 can be progressively locked in position via the ratchet. The second sheath 113 can be unlocked with a lever or a trigger. For example, the second sheath 113 can be unlocked by pushing the control 132 inward which can disengage the ratchet. The control 132 can interface with the second sheath 113, for example, via the actuator 115. For example, a first end of the actuator 115 can be connected to the control 132 and a second end of the actuator 115 can be connected to the distal end of the second sheath 113. The control 132 can be connected to a spool that the first end of the actuator 115 can be wound and unwound around. For example, raising the distal end of the second sheath 113 into the space 105 (e.g., via clockwise rotation of the control 132) can include applying tension to the actuator 115 and winding the first end of the actuator 115 around the spool, and lowering the distal end of the second sheath 113 back into the device 100 (e.g., via counterclockwise rotation of the control 132) can include releasing tension from the actuator 115 and unwinding the first end of the actuator 115 from the spool.

FIG. 30A illustrates that the device 100 can have a device length 100_{L} of about 24 inches to about 48 inches, including every 1 inch increment within this range (e.g., 24 inches, 36 inches, 42 inches, 48 inches). FIG. 30A illustrates the device length 100_{L} when the device 100 is in a straight configuration. The length of the handle 130 can be about 5 inches to about 10 inches, including every 1 inch increment within this range.

FIG. 30A illustrates that a tool (e.g., the endoscope 121) can be advanced and withdrawn through the device 100, for example, through the first sheath 101, through the fourth sheath 141, and through the handle 130. As another example, a tool (e.g., the third sheath 117 and the guidewire 119) can be advanced and withdrawn through the device 100, for example, through the second sheath 113, through the first sheath 101, and through the handle 130.

FIG. 30A illustrates the engager 106 in an expanded configuration with the distal tip of the second sheath 113 in a deflected configuration in the space 105.

FIG. 30A illustrates that the device 100 can have a distal end 127. The distal end 127 can be atraumatic. FIG. 30A illustrates that the distal end 127 can be tapered, which can make the distal end 127 atraumatic. FIG. 30A illustrates that the distal-most sheath segment 128_{S} can be the distal end 127 of the device 127 and that the distal-most sheath segment 128_{S} can be tapered.

FIG. 30B illustrates that the sheath segments 128_{S} can have a sheath segment length 128_{SL} of about 10 mm to about 50 mm, including every 1 mm increment within this range (e.g., 10 mm, 30 mm, 50 mm). FIG. 30B illustrates that the sheath segments 128_{S} can have a sheath segment height 128_{SH} of about 10 mm to about 30 mm, including every 1 mm increment within this range (e.g., 10 mm, 20 mm, 30 mm). For example, FIG. 30B illustrates that the sheath segments 128S can have a sheath segment length 128_{SL} of 30 mm and a sheath segment height 128_{SH} of about 20 mm.

The dimensions shown in FIG. 30B can be varied. For example, as shown in FIG. 30A, the distal most sheath segments 128S (e.g., the distal most 1 to 4 sheath segments 128S) can have varied dimensions relative to the more proximal sheath segments 128S to accommodate the features at the distal tip of the device 100 (e.g., the engager 106, the first opening 102a, the second opening 102b, the third opening 102c. For example, FIG. 30A illustrates that the three distal most sheath segments 128_{S} can have different dimensions than the rest of the sheath segments 128_{S}. As another example, all of the sheath segments 128_{S}, including the distal most sheath segments 128_{S}, can have the same dimensions (e.g., a sheath segment length 128_{SL} of 30 mm and a sheath segment height 128_{SH} of about 20 mm).

FIG. 30B illustrates that when the first sheath 101 is in a straight configuration, the space 129 between the sheath segments 128_{S} can have a length 129_{L}, for example, of about 1 mm to about 20 mm, including every 1 mm increment within this range (e.g., 1 mm, 5 mm, 10 mm, 20 mm). For example, FIG. 30B illustrates that the length 129_{L} between two adjacent sheath segments 128_{S} can be about 5 mm. FIGS. 30A and 30B further illustrates that the length 129_{L} of the spaces 129 between the sheath segments 128_{S} can be uniform. For example, the length 129_{L} can be the same between all the sheath segments 128_{S}. The spaces 129 can be contractible and expandable. This can allow the first sheath 101 to bend, and can allow the first sheath 101 to bend the second and fourth sheaths 113, 141. For example, when the first sheath 101 is bent in a first direction, a first end of the spaces 129 can contract (e.g., such that the length 129_{L} is reduced or becomes zero) and a second end of the spaces 129 can expand (e.g., such that the length 129_{L} is increased). The length 129_{L} on the contraction side can decrease by the full length of the length 129_{L} when the first sheath 101 is bent into a curved configuration. For example, where the length 129_{L} is about 5.0 mm, the contraction side can decrease, for example, by 0.1 mm to about 5.0 mm, including every 0.1 mm increment in this range when the first sheath 101 is bent into a curved configuration. The length 129_{L} on the expansion side can increase, for example, by 0.1 mm to about 7.0 mm, including every 0.1 mm increment in this range when the first sheath 101 is bent in a curved configuration. FIG. 30K, described in greater detail below, illustrates an exemplary configuration of contracted and expanded spaces 129, whereby the length 129_{L} of the spaces 129 can be greater on a first side of the first sheath 101 than on a second side of the first sheath 101 when the first sheath 101 is in a curved configuration (e.g., the curved configuration shown in FIG. 30K). In the curved configuration shown in FIG. 30K, the first side of the first sheath 101 can be on the convex side of the curve and the second side of the first sheath 101 can be on the concave side of the curve.

FIG. 30C illustrates that the control 132 can extend through both sides of the handle 130 to accommodate left-handed users, right-handed users, and/or so that one or multiple fingers can be used to turn the control 132. FIG. 30C illustrates an exemplary expanded configuration of the engager 106. FIG. 30C illustrates that the stabilizers 107 can extend past the width of the first sheath 101 when the engager 106 is in an expanded configuration. FIG. 30C illustrates that the expander 109 can extend past the outer lateral edges of the stabilizers 107 when the engager 106 is in an expanded configuration.

FIG. 30D illustrates that the sheath segments 128_{S} can have a sheath segment width 128_{SW} of about 10 mm to about 30 mm, including every 1 mm increment within this range (e.g., 10 mm, 20 mm, 30 mm). For example, FIG. 30D illustrates that the sheath segments 128_{S} can have a sheath segment width 128_{SW} of about 20 mm. FIGS. 30A-30D illustrate that the sheath segment height 128_{SH} the sheath segment width 128_{SW} can be the same.

The dimensions shown in FIGS. 30A-30D can be varied, for example, in the distal most 1 to 4 sheath segments 128_{S} to accommodate, for example, the features of the engager 106, the first opening 102a, the second opening 102b, the third opening 102c, or any combination thereof. FIGS. 30A-30D illustrate, for example, that the dimensions of some of the more distal sheath segments 128_{S} can be larger or smaller than the dimensions of some of the more proximal sheath segments 128_{S}. As another example, one or multiple dimensions of the sheath segments 128 can become progressively smaller from the proximal end of the first sheath 101 to the distal end of the first sheath 101, for example, to make the distal end of the first sheath 101 more flexible (e.g., capable of a smaller radius of curvature) than the proximal end of the first sheath 101. For example, the sheath segment length 128_{SL} can be progressively made shorter by about 0.1 mm to about 2.0 mm every sheath segment 128_{S} or after every consecutive group of 2 to 5 sheath segments 128_{S}. As the sheath segment length 128_{SL} is made shorter from proximal end of the first sheath 101 to the distal end of the first sheath 101, the sheath segment height 128_{SH} and the sheath segment width 128_{SW} can remain the same. As the sheath segment length 128_{SL} is made shorter from proximal end of the first sheath 101 to the distal end of the first sheath 101, the length 129_{L} between adjacent sheath segments 128_{S} can stay constant or can become progressively larger, for example, by 0.1 mm to about 2.0 mm every sheath segment 128_{S} or after every consecutive group of 2 to 5 sheath segments 128s.

FIG. 30E illustrates that the stabilizers 107 can have a stabilizer first end 107a and a stabilizer second end 107b. The first end 107a can be the proximal end of the stabilizer 107 and the second end 107b can be the distal end of the stabilizer 107. FIG. 30E illustrates that one end of the stabilizers 107 can be moveable relative to the other end of the stabilizers 107 when the stabilizers 107 are expanded, for example, via the expander 109. For example, FIG. 30E illustrates that when the stabilizers 107 are expanded, the stabilizer first ends 107a can be moved toward the stabilizer second ends 107b in direction 134a as the medial portions of the stabilizers 107 are moved outward, for example, by the expander 109 as the expander 109 is expanded. FIG. 30E illustrates that when the stabilizers 107 are contracted (e.g., from the expanded configuration shown in FIG. 30E), the stabilizer first ends 107a can be moved away from the stabilizer second ends 107b in direction 134b as the medial portions of the stabilizers 107 move inward, for example, as the expander 109 is contracted. The stabilizer first and second ends 107a, 107b can be attached to the first sheath 101, for example, via connectors 107_{C} (e.g., pins, rivets, rods, glue). For example, the stabilizer first and second ends 107a, 107b can be attached to sheath segments 128_{S}. The stabilizer second end 107b can be attached to the distal tip 127.

The stabilizer second ends 107b can be in a fixed position relative to the stabilizer first ends 107a, or vice versa. The stabilizer first ends 107a can be moveable relative to the stabilizer second ends 107b, or vice versa. FIG. 30E illustrates that the stabilizer first ends 107a can move toward and away from the stabilizer second ends 107b as the stabilizers 107 are expanded and contracted, respectively. FIG. 30E illustrates, for example, that the stabilizer first ends 107a can be slidably connected to the connectors 107_{C} and that the stabilizer second ends 107b can be non-slidably connected to the connectors 107_{C}. FIG. 30E illustrates that the stabilizer first ends 107a can have a stabilizer slot 107_{S} that can slide on the connectors 107_{C} in directions 134a and 134b when the stabilizers are expanded and contracted, respectively. Thus, when the expander 109 is expanded, the stabilizer first ends 107a can slide on the connector 107_{C} toward the stabilizer second ends 107b, and when the expander 109 is contracted, the stabilizer first ends 107a can slide on the connector 107_{C} away from the stabilizer second ends 107b. This relative movement between the stabilizer first and second ends 107a, 107b can advantageously allow the expander 109 to expand and contract the stabilizers 107. When the engager 106 is expanded and contracted, the length of the distal end of the device 100 (e.g., as measured between the proximal and distal connectors 107_{C}) can stay the same. When the engager 106 is expanded and contracted, the device length 100_{L} can stay the same. The stabilizer first ends 107a can be closer to a distal terminal end of the device 100 (e.g., to the distal terminal end of the first sheath 101) when the stabilizers 107 are in an expanded configuration than when the stabilizers 107 are in the unexpanded configuration. As another example, the stabilizer first ends 107a can be fixed and the stabilizer second ends 107b can be moveable (e.g., the opposite of what is described above and shown in FIG. 30A). As yet another example, both the stabilizer first and second ends 107a, 107b can be moveable on the connectors 107_{C}.

FIG. 30F illustrates that the device 100 can have a torque carrier 136 that can transmit torque applied to a proximal end of the device 100 to a distal end of the device 100. The torque carrier 136 can allow rotational motion applied to the proximal end of the device 100 to be transmitted to the distal end of the device 100 so that the distal tip of the device 100 (e.g., the portion having the engager 106) can be navigated to the target by turning the distal tip while the distal tip is in the body and the handle 130 is outside of the body. The distal end of the torque carrier 136 can rotate the same or fewer degrees as the proximal end of the torque carrier 136 when torque is applied to the proximal end of the torque carrier 136. The torque carrier 136 can transmit torque when the torque carrier 136 is in a straight configuration. The torque carrier 136 can transmit torque when the torque carrier 136 is in a curved configuration. Torque can be applied to the torque carrier 136 by rotating the torque carrier 136 about the device longitudinal axis A₁, for example, in directions 137a and 137b. Torque can be applied to the torque carrier 136 by rotating the torque carrier 136 perpendicular to the device longitudinal axis A₁, for example, in directions 137c and 137d. The torque carrier 136 can be flexible to bend but rigid to transmit torque. The torque carrier 136 can be attached to the first sheath 101 so that torque can be transmitted from the torque carrier 136 to the first sheath 101. Torque can be transmitted from the torque carrier 136 to the first sheath 101 wherever the torque carrier 136 is attached to the first sheath 101, also referred to as attachment points. The sheath segments 128_{S} can allow the first sheath to be flexible 101 and the torque carrier 136 can transmit torque to the distal end of the first sheath 101. The torque carrier 136 can extend from a proximal end of the device 100 to the distal end of the device 100. For example, the torque carrier 136 can have the length 101_{L}, for example, less the length of the handle 130.

FIG. 30F illustrates that the torque carrier 136 can be a jacket 136a that can be attached (e.g., glued) to the first sheath 101, such as to each of the sheath segments 128_{S}. The jacket 136a can be a tube. The tube can be a solid tube. The jacket 136a can be an outer tube. For example, the jacket 136a can bonded to the face of each of the sheath segments 128_{S}. The connection of the torque carrier 136 to the first sheath 101 can allow for torque applied to the torque carrier 136 to be transmitted to the first sheath 101, for example, from the proximal end of the torque carrier and first sheath 136, 101 to the distal end of the torque carrier and first sheath 136, 101. Thus, turning or moving the torque carrier 136 can turn or move the first sheath 101 (e.g., the segments 128_{S}). The proximal terminal end of the first sheath 101 and the torque carrier 136 (e.g., the jacket 136a) can be attached to the handle 130. FIG. 30F illustrates, for example, that the first sheath 101 and the torque carrier 136 (e.g., the jacket 136a) can extend into the handle 130, which is shown transparent. The handle 130 can thus transmit torque to the torque carrier 136. In this way, the torque carrier 136 can transmit torque from the handle 130 to the distal tip of the device to steer the device 100, for example, by turning the handle 130 clockwise or counterclockwise about or perpendicular to the device longitudinal axis A₁. Torque can be applied to the device 100 in any direction. For example, torque applied to the torque carrier 136 (e.g., via the handle 130) in direction 137a (e.g., clockwise direction) about the device longitudinal axis A₁ can be transmitted through the torque carrier 136 (e.g., through the jacket 136a) and cause the distal tip of the device 100 to rotate in direction 137a. As another example, torque applied to the torque carrier 136 (e.g., via the handle 130) in direction 137b (e.g., counterclockwise direction) about the device longitudinal axis A₁ can be transmitted through the torque carrier 136 (e.g., through the jacket 136a) and cause the distal tip of the device 100 to rotate in direction 137b. Pushing the device 100 through curved lumens can naturally cause the device 100 to bend. The device 100 can naturally bend as the device 100 is advanced through curved lumens. The device 100 can naturally bend to conform to the shape of the lumen the device 100 is in as the device 100 is pushed into the body. For example, as the device 100 is advanced in the body, the body (e.g., lumen wall, stomach wall) can push back against the device 100, which can cause the device 100 to take on a curved configuration. For example, as the distal tip of the device 100 encounters tissue of a lumen wall, the distal end of the device 100 can become curved as the distal tip of the device 100 deflects off the lumen wall. For example, the device 100 can become curved as the distal tip of the device 100 encounters resistance and continues to be advanced through bends in the body. This ability of the device 100 to curve around bends in the body (e.g., from the esophagus to the Ampulla of Vater 123) as the device 100 is advanced in the body to the target can advantageously allow the device 100 to be advanced to the target without damaging tissue or to inhibit the damaging of tissue as the device 100 is advanced. By rotating the device in directions 137a and 137b when the device 100 is in the body, the user can point the distal tip of the device 100 in the direction the user would like to further advance the device 100. As needed, the device 100 can be rotated in directions 137a and 137b to direct the distal tip of the device 100 to the next lumen or to the target location. As yet another example, torque applied to the torque carrier 136 (e.g., via the handle 130) in a direction perpendicular to the device longitudinal axis A₁, for example, in direction 137c, can be transmitted through the torque carrier 136 (e.g., through the jacket 136a) and cause the distal tip of the device 100 to move in the opposite direction, for example, in direction 137d. As still yet another example, torque applied to the torque carrier 136 (e.g., via the handle 130) in a direction perpendicular to the device longitudinal axis A₁, for example, in direction 137d, can be transmitted through the torque carrier 136 (e.g., through the jacket 136a) and cause the distal tip of the device 100 to move in the opposite direction, for example, in direction 137c. FIG. 30F illustrates that the device longitudinal axis A₁ can be a center longitudinal axis of the first sheath 101 (e.g., of the sheath segments 128_{S}) and that it can be offset from the center longitudinal axes of the first and second sheaths 101, 113 (e.g., between them as shown in FIG. 30F). As still yet additional examples, toque can be applied to the torque carrier (e.g., via the handle 130) in directions perpendicular to the device longitudinal axis A₁ for example, in directions into and out of the plane of FIG. 30F (e.g., in directions perpendicular to the device longitudinal axis A₁, direction 137c, and direction 137d). As these exemplary torques show, torque can be applied to the torque carrier 136 (e.g., via the handle 130) in any direction to cause the desired movement of the distal tip of the device 100. In these exemplary ways, torque can be transmitted from the proximal most sheath segment 128_{S} to the distal most sheath segment 128s.

The torque carrier 136 (e.g., jacket 136a) can be opaque or transparent. In FIG. 30F, the torque carrier 136 (e.g., jacket 136a) is shown transparent. The jacket 136a can have the same cross-sectional shape as the first sheath 101. The jacket 136a can have a thickness of about 0.10 mm to about 2.00 mm, including every 0.10 mm increment within this range (e.g., 0.10 mm, 1.00 mm, 1.50 mm, 2.00 mm). The torque carrier 136 can allow the device 100 to be rotated in the body.

FIG. 30G illustrates a close-up of the torque carrier (e.g., the jacket 136a) attached to the first sheath 101.

FIG. 30H illustrates an exemplary distal tip of the device 100 when the engager 106 is in an unexpanded configuration and when the second sheath 113 is in a deflected configuration. FIG. 30H illustrates that the second sheath 113 can be deflected into a curved configuration when the engager 106 is in the unexpanded configuration. As another example, FIG. 30H illustrates that the engager 106 can be contracted before the second sheath 113 is lowered.

FIG. 30I illustrates the handle 130 transparent so that the control 132 can be seen through the handle 130, extending through both lateral sides of the handle 130.

FIG. 30J illustrates that the first sheath 101, the second sheath 113, and the first sheath 101 can extend into the handle 130. For example, FIG. 30J illustrates that the handle 130 can have a connector 133 that the first sheath 101, the second sheath 113, the first sheath 101, and/or the torque carrier 136 can extend into and/or be connected to. For illustrative purposes, the first sheath 101, the handle 130, and the torque carrier 136 (e.g., the jacket 136a in FIG. 30J) are shown transparent.

FIG. 30K illustrates the device 100 in an exemplary curved configuration that can be caused by applying torque to the device 100 (e.g., by rotating the handle 130 about the device longitudinal axis A₁ and/or about axes perpendicular to the device longitudinal axis A₁). Applying torque to the torque carrier 136 (e.g., the jacket 136a) can allow the user to steer the device 100 to the target (e.g., the Ampulla of Vater 123), for example, by pointing the distal tip of the device 100 toward the lumen the user would like to enter and then advancing the device 100, for example, by pushing on the handle 130 in a direction parallel to the device longitudinal axis A₁. The device 100 can take on the various curved configurations of the lumens the device 100 is advanced through in the body.

As shown in FIG. 30K, the torque carrier 136 can be the jacket 136a. The jacket 136a can be an outer jacket attached to the outer surfaces of the sheath segments 128_{S}. The jacket 136a can be an external. In FIG. 30K, the jacket 136a is shown opaque, and the sheath segments 128_{S} and the spaces 129 are labeled according to where these features are under the jacket 136a. FIG. 30K further illustrates that the spaces 129 have tapered shapes, with the spaces 129 schematically shown between two dashed lines as having, for example, triangular shapes, cut pie shapes, truncated triangular shapes (e.g., trapezoids), and/or truncated cut pie shapes. FIG. 30K illustrates that when the first sheath 101 is in a curved configuration, the space 129 can be compressed on the inner radial side (e.g., the side with the narrower portion of the space 129) and can be expanded on the outer radial side (e.g., the side with the wider portion of the space 129). FIG. 30K thus illustrates that the spaces 129 can expand and contract as the device is bent into curved configurations. The spaces 129 can return to the dimensions shown in FIGS. 30A-30D when the first sheath 101 is returned to a straight configuration. FIG. 30K illustrates that when the device 100 is in a curved configuration (e.g., the curved configuration shown in FIG. 30K), the device 100 can have a radius of curvature 138. The smallest radius of curvature 138 that the device 100 can have can be from about 5.0 mm to about 15.0 mm, including every 1.0 mm increment within this range (e.g., 5.00 mm, 10.0 mm, 15.0 mm). For example, FIG. 30K illustrates that the smallest radius of curvature 138 of the first sheath 101 can be about 10.0 mm. When the radius of curvature 138 is the smallest radius of curvature, adjacent sheath segments 128_{S} may or may not contact each other on the inner radial side. In cases where adjacent sheath segments 128_{S} contact each other on the inner radial side, such contact can be a safety feature that allows the sheath segments 128_{S} to inhibit or prevent over bending of the device 100, for example, beyond the smallest radius of curvature permitted by the device 100.

FIG. 31A illustrates that the torque carrier 136 can extend through the first sheath 101, for example, through a lumen in each of the sheath segments 128_{S}. FIG. 31A illustrates that the torque carrier 136 can be an extension 136b, for example, a cable, a rod, or a wire that can be attached (e.g., glued) to the first sheath 101, such as to an inner surface of each of the sheath segments 128_{S}. FIG. 31A illustrates that the torque carrier 136 may not have a lumen. For example, the extension 136b may not have a lumen. FIG. 31A illustrates that a jacket 139 can be attached to the outer surfaces of the sheath segments 128_{S}. The jacket 139 may or may not carry torque with the torque carrier 136. The jacket 139 can be different from the jacket 136a discussed with reference to FIGS. 30F-30J. For example, FIG. 31A illustrates that the jacket 139 may not carry torque. For example, in FIG. 31A, the jacket 139 may not be bonded to the face of each of the sheath segments 128_{S} so that the extension 136b (e.g., cable, rod, or wire) in a lumen of the first sheath 101 can be the primary or sole torque carrier 136. The jacket 139 can be on the outside of the device 100 so that there is a smooth transmission of torque between each sheath segment 128_{S}. The jacket 139 and the sheath segments 128_{S} are shown transparent in FIG. 31A so that the torque carrier 136 (e.g., the extension 136b) can be seen in the figure. As shown in FIG. 31A, the proximal terminal end of the first sheath 101 and the torque carrier 136 (e.g., the extension 136b) can be attached to the handle 130. FIG. 31A illustrates, for example, that the first sheath 101 and the torque carrier 136 (e.g., the extension 136b) can extend into the handle 130, which is shown transparent. Applying torque to the torque carrier 136 (e.g., via the handle 130) can deflect the distal tip of the device 100 in the desired direction.

FIG. 31B illustrates that lumens 140 can extend through the first sheath 101, for example, through the sheath segments 128_{S}. The first sheath 101 can have multiple lumens 140, for example, lumens 140a, 140b, 140c, and 140d. FIG. 31B illustrates that the fourth sheath 141 can extend through the lumen 140a. The torque carrier 136 (e.g., extension 136b) can extend through the lumen 140b. The lumen 140c can be an inflation and deflation lumen for the expanders 109. The lumen 140d can be an auxiliary lumen that can be used to insert tools into the device 100 and advance them to the distal tip of the device 100.

FIG. 31C illustrates that the first sheath 101 can include lumens 140a-140i. The second sheath 113, the third sheath 117, and the guide wire 119 can extend through the lumen 140e. The lumen 140h can be an inflation and deflation lumen for the expanders 109. The lumens 140f, 140g, and 140i can be auxiliary lumens that can be used to insert tools into the device 100 and advance them to the distal tip of the device 100. As another example, the actuators 115 can extend through the lumen 140e. As another example, the actuators 115 can extend through the lumen 140f. As another example, the device 100 can have two torque carriers 136 (e.g., two wires, rods, cables), one in the lumen 140b and another in the lumen 140i. As yet another example, the torque carrier 136 shown in FIG. 31A can be in the lumen 140f instead of the lumen 140b. As yet still another example, the lumen 140a can be the first sheath first lumen 101_{L1} and the lumen 140e can be the first sheath second lumen 101_{L2}.

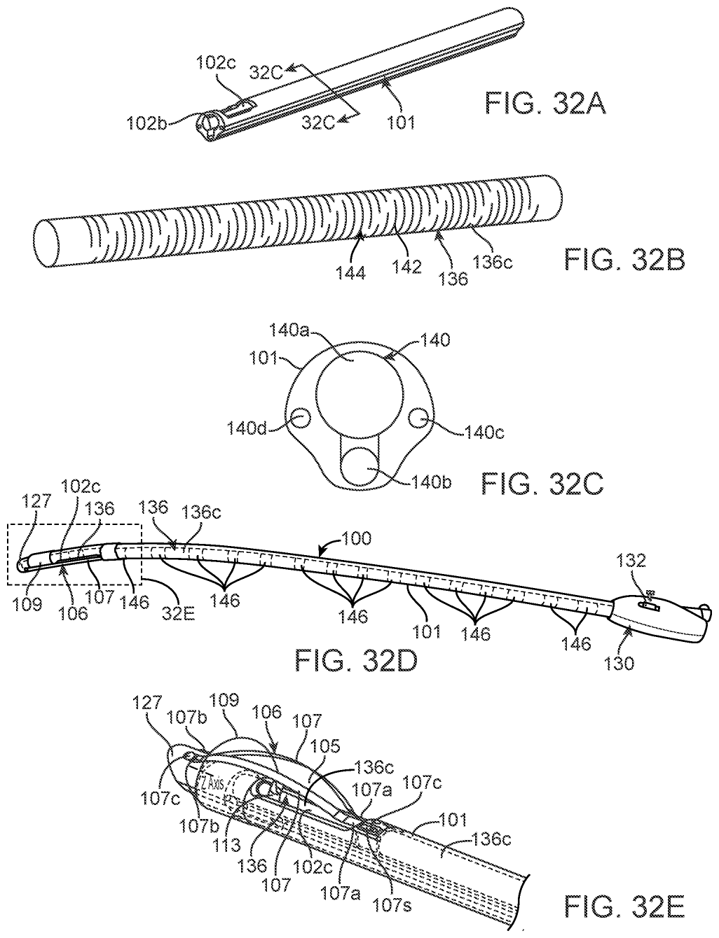

FIG. 32A illustrates that the first sheath 101 can be a continuous sheath, for example, a non-segmented sheath (e.g., in contrast to the segmented first sheath 101 illustrated in FIGS. 30A-30K). A distal tip (e.g., the distal tip 127 having a taper and/or a tip that is atraumatic) can be attached to the distal end of the first sheath 101 shown in FIG. 32A. The first sheath 101 can be, for example, extruded to the desired length (e.g., to the length 100_{L} less the length of the distal tip and the length of the handle 130). The first sheath 101 can have the second and third openings 102b, 102c. A torque carrier 136 (e.g., the jacket 136a) can be attached to the outer surface of the first sheath 101. As another example, the jacket 139 can be attached to the outer surface of the first sheath 101. As explained further below, a torque carrier 136 different from the jacket 136a (e.g., a tube of material) or the extension 136b (e.g., wire, cable, or rod) can be attached to the first sheath 101.

FIG. 32B illustrates that the torque carrier 136 can be, for example, a tube 136c. The tube 136c can be an inner tube 136c, for example, placeable in a lumen of the first sheath 101. The tube 136c can be cylindrical. The tube 136c can have openings 142. The openings 142 can define an interrupted spiral 144 in the wall of the tube 136c. The tube 136c can be cut to create the openings 142. The solid sections of the tube 136c between the openings 142 can transmit force, for example, torque from a proximal end of the tube 136c to a distal end of the tube 136c. For example, the torque carrier 136 (e.g., the tube 136c) can be laser cut to have an interrupted spiral 144. The tube 136c can be flexible to bend but rigid to transmit torque. The tube 136c can be metal, such as stainless steel. The wall of the tube 136c can be about 0.2 mm to about 2.5 mm thick, including every 0.1 mm increment within this range (e.g., 0.2 mm, 0.5 mm, 1.0 mm, 2.5 mm). The interrupted spiral 144 in the wall of the tube 136c can give the tube 136c flexibility to allow it to bend while also allowing the tube 136c to transmit torque from the proximal end of the tube 136c to the distal end of the tube 136c. The fourth sheath 141 can extend through the torque carrier 136 (e.g., through the tube 136c in FIG. 32B). As another example, the torque carrier 136 (e.g., the tube 136c in FIG. 32B) can be in the fourth sheath 141. As another example, the torque carrier 136 (e.g., the tube 136c) can be the fourth sheath 141. For example, the tube 136c can provide a stable lumen for the endoscope 121 to be moved in directions 121a and 121b.

FIG. 32C illustrates that the first sheath 101 can have lumens 140, for example, lumens 140a-140d. The lumen 140a can be for the torque carrier 136 (e.g., the tube 136c in FIG. 32B) and the endoscope 121. The outer surface of the tube 136c can be attached (e.g., glued) to the inner surface of the first sheath 101 that defines the lumen 140a. The lumen 140a can be for the first sheath 101 and the endoscope 121. The lumen 140a can be the first sheath first lumen 101_{L1}. The lumen 140b can be for the second sheath 113. The second sheath 113 can extend through the lumen 140b. The lumen 140b can be the first sheath second lumen 101_{L2}. The lumens 140c and 140d can be inflation and deflation lumens that can be connected to the expanders 109. The lumens 140c and 140d can inflate and deflate in parallel. As another example, the lumen 140c can be an inflation lumen and the lumen 140d can be a deflation lumen, or vice versa.

FIG. 32D illustrates a variation of the device 100 having the torque carrier 136 when the torque carrier 136 is the tube 136c (e.g., the tube 136c shown in FIG. 32D). As shown in FIG. 32D, the first sheath 101 can be attached to the handle 130. FIG. 32D illustrates that the torque carrier 136 can be attached to the first sheath 101 at attachment points 146, for example, via glue. Torque can be transmitted from the torque carrier 136 (e.g., the tube 136c) at the attachment points 146. The first sheath 101 in FIG. 32D is shown transparent.

FIG. 32E illustrates an exemplary distal tip of the device 100 when the engager 106 is in an expanded configuration and when the second sheath 113 is in a deflected configuration. FIG. 32E illustrates that an opening can be cut in the torque carrier (e.g., the tube 136c) so that the third opening 102c can extend through both the first sheath 101 and the torque carrier 136. As FIG. 32E illustrates, when the second sheath 113 is in a deflected configuration, the second sheath 113 can extend through the sheath 101 and the torque carrier 136 (e.g., the tube 136c), for example, through the third opening 102c. In FIG. 32E, the expander 109 (e.g., a balloon) is shown transparent for illustrative purposes so that the full length of both of the stabilizers 107 illustrated in FIG. 32E across the space 105 can be seen. For example, in FIG. 32E, the stabilizers 107 are shown in contact with an outer surface of the expander 109 (e.g., in contact with an outer surface of the balloon) when the expander 109 is in an expanded configuration. Because the expander 109 is shown transparent in FIG. 32E, the bottom surface (e.g., the portion of the stabilizer 107 is contact with the expander 109) can be seen through the expander 109. FIG. 32E illustrates that a portion of the expander 109 (e.g., a medial portion of the expander 109) can extend through the space between the two stabilizers 107 when the expander 109 is in an expanded configuration. As another example, the expander 109 may not extend through the space between the two stabilizers 107 such that the outer surface of the expander 109 is flush with the outer surface of the stabilizers 107 or is below the outer surface of the stabilizers 107 (e.g., closer to the device longitudinal axis A₁) when the expander 109 is in an expanded configuration.

FIGS. 30A-32E illustrate that the torque carrier 136 can be, for example, a jacket 136a, an extension 136b (e.g., rod, cable, wire), a tube 136c, or any combination thereof. As another example, the torque carrier 136 can be an inner jacket. As another example, the torque carrier 136 can be a coil or a braid attached to or integrated with the first sheath 101 in FIG. 32A to transmit torque.

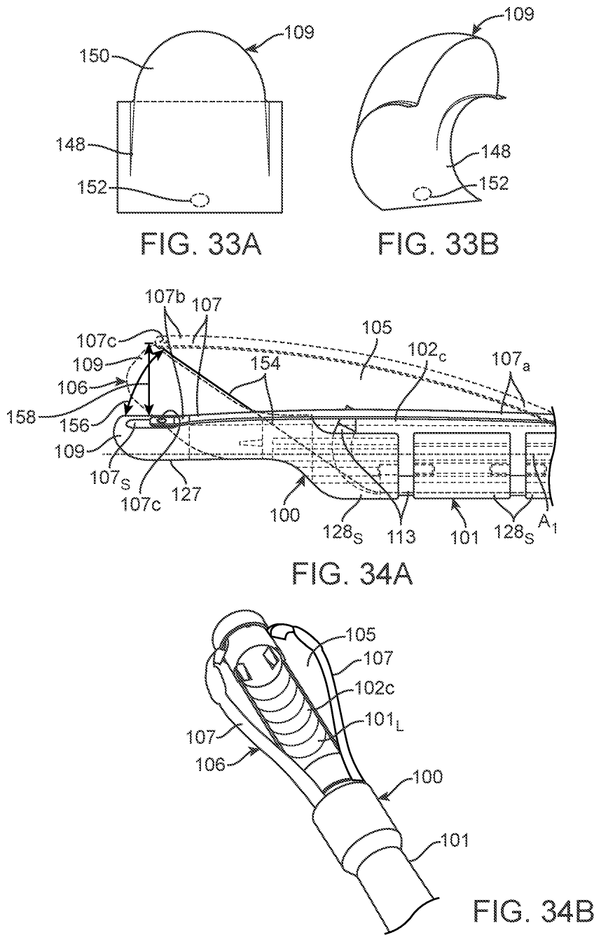

FIG. 33A illustrates that the expander 109 can be a double-layer balloon, for example, with a first layer 148 and a second layer 150. A space between the first and second layers 148, 150 can be inflatable and deflatable. The first layer 148 can be non-expandable such that the first layer 148 does not expand and contract with the expander 109 is inflated and deflated. The second layer 150 can be expandable such that the second layer 150 can expand away from the first layer 150 as the space between the first and second layers 148, 150 is inflated. The second layer 150 can be contractible such that the second layer 150 can contract toward the first layer 150 as the space between the first and second layers 148, 150 is deflated. The first layer 148 can wrap around the device 100. For example, the first layer 148 can have a cylindrical shape with a port 152 that allows you to inflate the space between the first and second layers 148, 150. The first layer 148 can be attached to the device 100, for example, to the first sheath 101, to the torque carrier 136 (e.g., to the jacket 136a), to the jacket 139, or any combination thereof. The port 152 can connect the inflation and deflation lumens to the space between the first and second layers 148, 150. The expander 109 in FIG. 33A is shown in an exemplary expanded configuration. The expanded configuration in FIG. 33A can be, for example, the expander 109 in a fully inflated configuration.

FIG. 33B illustrates that the expander 109 can be a single-layer balloon, having only the first layer 148. A first portion of the first layer 148 can be connected to the device 100, for example, to the first sheath 101, to the torque carrier 136 (e.g., to the jacket 136a), to the jacket 139, or any combination thereof. A second portion of the first layer 148 can expand and contract as a space between the expander and the device 100 is inflated and deflated, for example, through the port 152. Half of the expander 109 (e.g., the balloon) is shown in FIG. 33B for illustrative purposes only.

FIG. 34A illustrates a variation of an engager 106. FIG. 34A illustrates, for example, that the engager 106 can include the distal tip of the device 100. For example, FIG. 34A illustrates that the expander 109 can be the distal tip of the device 100. The distal tip of the device 100, also referred to as the expander 109 in reference to FIG. 34A, can move the stabilizer 107 from a contracted configuration to an expanded configuration (e.g., from a stabilizer first position to a stabilizer second position), and vice versa. For example, FIG. 34A illustrates that the distal tip of the device 100 (e.g., the expander 109) can be can be deflectable. FIG. 34A illustrates that the expander 109 can be the distal tip of one of the sheaths of the device 100, for example, the distal tip of the first sheath 101. Deflecting the distal tip of the first sheath 101 outward (e.g., away from the device longitudinal axis A₁) can cause the stabilizers 107 to expand which can create the space 105 and give the separation between the device tip and the mucosa to facilitate cannulation. For example, FIG. 34A illustrates a variation of an expanded configuration and contracted configuration of the engager 106. The expanded configuration is represented by the dashed lines in FIG. 34A, and is shown superimposed over the contracted configuration to show the relative positions of the engager 106 in the expanded configuration relative to the contracted configuration. FIG. 34A illustrates that the expander 109 can be a distal tip of the first sheath 101. The expander 109 can be a deflectable portion of the first sheath 101.

FIG. 34A illustrates that the distal tip of the device 100 (e.g., of the first sheath 101) can have a contracted configuration (also referred to as a non-deflected configuration) and an expanded configuration. When the distal tip of the device 100 (e.g., of the first sheath 101) is moved from the contracted configuration to the expanded configuration, the distal tip of the device 100 can move through a deflection angle 156 of about 1 degree to about 120 degrees, or more narrowly from about 1 degree to about 90 degrees, or still more narrowly from about 1 degree to about 75 degrees, including every 1 degree increment within these ranges (e.g., 1 degree, 75 degrees, 90 degrees, 120 degrees). The deflection angle 156 can be measured as shown in FIG. 34A. The deflection angle 156 can be the angle between the device longitudinal axis A₁ and a longitudinal axis that extends through the distal tip of the device 100 when in the distal tip is in the expanded configuration (e.g., the angle between a longitudinal axis through the dashed portion of the distal tip in FIG. 34A and a longitudinal axis through the solid portion of the distal tip in FIG. 34A). For example, 34A illustrates the deflection angle 156 as measured between a portion of the outer surface of the device 100 (e.g., of the first sheath 101) when the device 100 is in the expanded configuration relative to when the device 100 is in the contracted configuration. For example, FIG. 34A illustrates that the distal tip of the device 100 (e.g., of the first sheath 101) at the deflection angle 156 of about 45 degrees. When the distal tip of the device 100 is in the expanded configuration, the space 105 can have the expanded dimension D_{E}. When the distal tip of the device 100 is in the contracted configuration, the space 105 can have the contracted dimension Dc. A lumen (e.g., the first sheath lumen 101_{L}) can extend through the deflectable distal tip of the device 100 such that when the distal tip is in a deflected configuration, a first portion of the lumen (e.g., a first portion of the first sheath lumen 101_{L}) can be angled relative to a second portion of the lumen (e.g., a second portion of the first sheath lumen 101_{L}). The first and second portions of the lumen can be at the deflection angle 156 relative to each other.

As another example, when the distal tip of the device 100 (e.g., of the first sheath 101) is moved from the contracted configuration to the expanded configuration, the distal tip of the device 100 can move away from the device longitudinal axis A₁ and/or away from the second sheath 113 by a deflection dimension 158 of about 1 mm to about 60 mm, or more narrowly from about 1 mm to about 35 mm, or still more narrowly from about 1 mm to about 20 mm, including every 1 mm increment within these ranges (e.g., 1 mm, 20 mm, 35 mm, 60 mm). The deflection dimension 158 can be measured as shown in FIG. 34A. As another example, the deflection dimension 158 can be measured between the device longitudinal axis A₁ and the portion of the distal tip of the device that is furthest from the device longitudinal axis A₁. As another example, the deflection dimension 158 can be measured between the device longitudinal axis A₁ and the portion of the distal tip in FIG. 34A where the stabilizer second end 107b attaches to the distal tip. The deflection dimension 158 can be measured along an axis perpendicular to the device longitudinal axis A₁. For example, the deflection dimension 158 can be the distance that the dashed portion of the distal tip in FIG. 34A is displaced away from the solid portion of the distal tip in FIG. 34A perpendicularly away from the device longitudinal axis A₁. For example, FIG. 34A illustrates that the distal tip of the device 100 (e.g., of the first sheath 101) at the deflection dimension 158 of about 20 mm. When the distal tip of the device 100 is in the expanded configuration, the space 105 can have the expanded dimension D_{E}. When the distal tip of the device 100 is in the contracted configuration, the space 105 can have the contracted dimension Dc.

FIG. 34A illustrates that the distal tip of the device 100 (e.g., the distal tip of the first sheath 101) can be deflected via one or multiple actuators 154 (e.g., one actuator 154, two actuators 154). The actuators 154 can be tension carriers such as pull wires that can extend back to the handle 130. Applying tension to the actuators 154 (e.g., pulling on a pull wire), for example, via a control on the handle 130, can deflect the distal tip of the device 100 to an expanded configuration (e.g., the expanded configuration shown in FIG. 34A), and releasing tension from the actuators 154 (e.g., releasing tension from a pull wire) can deflect the tip back to a contracted configuration (e.g., the contracted configuration shown in FIG. 34A). The distal tip of the device 100 can thus be deflected in the same or a similar way as the second sheath 113, for example, using different actuators (e.g., using actuators 154 as opposed to actuators 115) and using a different control on the handle 130. FIG. 34A illustrates that the distal end of the actuators 154 (e.g., pull wires) can be attached to the distal end of the device 100. For example, the distal end of the actuators 154 (e.g., pull wires) can be attached to the stabilizer second ends 107b and/or to the distal tip of the device 100, such as to the distal tip of the first sheath 101. As another example, the distal end of the actuators 154 (e.g., pull wires) can be attached to the stabilizer first ends 107a. FIG. 34A illustrates, for example, that the distal end of the actuators 154 (e.g., pull wires) can be attached to the distal end of the first sheath 101 and/or to the stabilizer second ends 107b. The actuators 154 can extend to the handle 130, for example, through a lumen in the first sheath 101. For example, FIG. 34A illustrates that the actuator 154 can extend through the same lumen as the second sheath 113 or through a lumen adjacent to the lumen that the second sheath 113 is in.

FIG. 34A illustrates that when the expander 109 (e.g., the distal tip of the first sheath 101) is in the deflected configuration that the expander can be in an expanded configuration. FIG. 34A illustrates that when the expander 109 is in the deflected configuration, the stabilizer second ends 102b can be farther from the device longitudinal axis A₁ than the stabilizer first ends 102a.

FIG. 34A illustrates that the expander 109 can be moved away from and toward the device longitudinal axis A₁. As another example, the expander 109 can be moved away from and toward the first sheath lumen (e.g., lumen 101_{L}). FIG. 34A illustrates, for example, that the distal end of the device 100 (e.g., of the first sheath 101) can be deflectable upward (e.g., away from the device longitudinal axis A₁) to provide the same functionality as a balloon.

FIG. 34A illustrates that as the distal tip of the device 100 is deflected, the stabilizer second ends 107b can slide in the stabilizer slot 107_{S}. As another example, the device 100 may not have stabilizer slots 107_{S}. In such cases, the stabilizer first and second ends 107a, 107b can be attached to the device 100 (e.g., to the first sheath 101) and may not slide when the distal end of the device 100 is deflected as shown in FIG. 34A. Thus, while FIG. 34A illustrates that the stabilizer seconds ends 107b may be slideable, the stabilizer second ends 107b may not be slideable.

FIG. 34A illustrates that the device 100 may not have a balloon.

FIG. 34A illustrates that the bulk of the distal end of the device 100 can be decreased, for example, relative to FIG. 1, to increase the visibility in the lumen (e.g., in the intestinal lumen 125). The shape of the distal end of the device 100 can, for example, advantageously increase the surface area in the lumen that is visible.

FIG. 34A illustrates that the distal tip of the device 100 can be atraumatic, and can have, for example, a tapered distal end. For example, the distal tip of the device 100 can have the snub-nose configuration shown in FIG. 34A.

FIG. 34B illustrates that the device 100 may not have a balloon. FIG. 34B illustrates that the second sheath 113 can be translated into and out of the first sheath lumen 101_{L}. For example, FIG. 34B illustrates the device 100 before the second sheath 113 is translated into the opening 102c in the first sheath lumen 101_{L}.

FIGS. 1-34B illustrate that the engager 106 (e.g., the stabilizers 107 and/or the expander 109) can extend partially around the circumference of the device 100, for example, partially around the perimeter of the first shaft 101 when the engager 106 is in an expanded configuration and when the engager 106 is in the unexpanded configuration. As another example, the engager 106 (e.g., the stabilizers 107 and/or the expander 109) can extend fully around the circumference of the device 100, for example, fully around the perimeter of the first shaft 101 one or multiple times when the engager 106 is in an expanded configuration and/or when the engager 106 is in the unexpanded configuration.

FIGS. 1-34B illustrate, for example, that the access device 100 can have a first sheath 101. The first sheath 101 can have a first sheath lumen (e.g., the first sheath lumen 101). The access device 100 can have a second sheath 113 having a second sheath lumen 113_{L}. The second sheath 113 can be deflectable into and out of the first sheath lumen 101. The second sheath 113 can have a deflected configuration and a non-deflected configuration. When the second sheath 113 is in the deflected configuration, a second sheath first portion can be in the first sheath lumen 101_{L1} and can extend across the first sheath lumen 101. When the second sheath 113 is in the non-deflected configuration, the second sheath first portion can be out of the first sheath lumen 101. The access device 100 can have an engager 106. The engager 106 can be expandable and contractible. The engager 106 can have an expanded configuration and a contracted configuration. When the engager 106 is in the expanded configuration, a space 105 can be between the engager 106 and the first sheath 101. When the engager 106 is in the expanded configuration, a second sheath second portion can be deflectable into the space 105. When the engager 106 is in the expanded configuration and when the second sheath 113 is in the deflected configuration, the second sheath second portion can be in the space 105. When the engager 106 is in the expanded configuration and when the second sheath 113 is in the non-deflected configuration, the second sheath second portion can be out of the space 105. The second sheath second portion can be a distal terminal end of the second sheath 113. When the second sheath 113 is in the non-deflected configuration, the second sheath first portion can be straight. When the second sheath 113 is in the deflected configuration, the second sheath first portion can be curved. When the second sheath 113 is in the deflected configuration, the second sheath first portion can be more curved than when the second sheath 113 is in the non-deflected configuration. When the second sheath 113 is in the deflected configuration, the second sheath first and second portions can define a hook shape. When the second sheath is in the non-deflected configuration, the second sheath first portion can be parallel to the first sheath lumen 101_{L1}. The first sheath 101 can have a first sheath second lumen (e.g., the first sheath second lumen 101_{L2}). The second sheath 113 can be in the first sheath second lumen 101_{L2}. The engager 106 can have a stabilizer 107. The stabilizer 107 can have an expanded configuration and a contracted configuration. The stabilizer 107 can be moveable from the contracted configuration to the expanded configuration. When the stabilizer 107 is in the expanded configuration, the stabilizer 107 can be farther from the first sheath 101 than when the stabilizer 107 is in the contracted configuration. The engager 106 can have an expander 109 and a stabilizer 107. The expander 109 can be expandable and contractible. The expander 109 can have an expanded configuration and a contracted configuration. When the expander 109 is in the expanded configuration, the engager 106 can be in the expanded configuration. When the expander 109 is in the contracted configuration, the engager 106 can be in the contracted configuration. The stabilizer 107 can have an expanded configuration and a contracted configuration. The stabilizer 107 can be moveable from the contracted configuration to the expanded configuration via the expander 109. When the stabilizer 107 is in the expanded configuration, the stabilizer 107 can be farther from the first sheath 101 than when the stabilizer 107 is in the contracted configuration. The expander 109 can include a balloon. The access device 100 can have an endoscope 121, a third sheath 117, and a guidewire 119. The endoscope 121 can be moveable in the first sheath lumen 101_{L1}. The third sheath 117 and the guidewire 119 can be moveable in the second sheath lumen 113_{L} when the second sheath 113 is in the deflected configuration. The access device 100 can have a third sheath (e.g., the fourth sheath 141) having a third sheath first lumen (e.g., lumen 140a) and a third sheath second lumen (e.g., lumen 140b, lumen 140e). For example, with reference to FIGS. 30A-34B, the first sheath 101 can be the first sheath, the second sheath 113 can be the second sheath, and the fourth sheath 141 can be the third sheath such that the first sheath 101 can extend through the fourth sheath 141 and such that the second sheath 113 can extend through the fourth sheath 141. As another example, with reference to FIGS. 30A-34B, the tube 136c can be the first sheath, the second sheath 113 can be the second sheath, and the first sheath 101 can be the third sheath. The first sheath 101 can be in the third sheath first lumen. The second sheath 113 can be in the third sheath second lumen. The first sheath 101 can be a torque carrier 136. The access device 100 can have a torque carrier 136 attached to the first sheath 101.

FIGS. 1-34B illustrate, for example, that the access device 100 can have a first sheath 101. The first sheath 101 can have a first sheath lumen (e.g., the first sheath lumen 101) and a first sheath distal tip. The first sheath distal tip can be moveable away from and toward the first sheath lumen 101_{L1}. The first sheath 101 can have a first sheath deflected configuration and a first sheath non-deflected configuration. When the first sheath 101 is in the first sheath deflected configuration, the first sheath distal tip can be farther from the first sheath lumen 101_{L1} than when the first sheath 101 is in the first sheath non-deflected configuration. The access device 100 can have a second sheath 113 having a second sheath lumen 113_{L}. The second sheath 113 can be deflectable into and out of the first sheath lumen 101. The second sheath 113 can have a second sheath deflected configuration and a second sheath non-deflected configuration. When the second sheath 113 is in the second sheath deflected configuration, a second sheath first portion can be inside the first sheath lumen 101_{L1} and can extend across the first sheath lumen 101. When the second sheath 113 is in the second sheath non-deflected configuration, the second sheath first portion can be outside the first sheath lumen 101_{L1}. The access device 100 can have a stabilizer 107. The stabilizer 107 can be expandable and contractible. The stabilizer 107 can have an expanded configuration and a contracted configuration. The stabilizer 107 can be moveable from the contracted configuration to the expanded configuration via the first sheath distal tip. When the first sheath 101 is in the first sheath non-deflected configuration, the stabilizer 107 can be in the contracted configuration. When the first sheath 101 is in the first sheath deflected configuration, the stabilizer 107 can be in the expanded configuration. When the stabilizer 107 is in the expanded configuration, the stabilizer 107 can be farther from a first sheath lumen 101_{L1} longitudinal axis than when the stabilizer 107 is in the contracted configuration.

The stabilizer 107 can have a stabilizer first end and a stabilizer second end. When the stabilizer 107 is in the expanded configuration, the stabilizer first end can be closer to the first sheath lumen 101_{L1} longitudinal axis than the stabilizer second end.

The access device 100 can have an endoscope 121, a third sheath 117, and a guidewire 119. The endoscope 121 can be moveable in the first sheath lumen 101_{L1}. The third sheath 117 and the guidewire 119 can be moveable in second sheath lumen 113_{L} when the second sheath 113 is in the deflected configuration.

When the stabilizer 107 is in the expanded configuration, a space 105 can be between the stabilizer 107 and the first sheath 101. When the stabilizer 107 is in the expanded configuration, a second sheath second portion can be deflectable into the space 105. When the stabilizer 107 is in the expanded configuration and when the second sheath 113 is in the deflected configuration, the second sheath second portion can be in the space 105. When the stabilizer 107 is in the expanded configuration and when the second sheath 113 is in the non-deflected configuration, the second sheath second portion can be out of the space 105.

FIGS. 1-34B illustrate, for example, a method of accessing a target in and/or from a body lumen. The method can include advancing a first sheath 101, a second sheath 113, and an engager 106 to the target. The first sheath 101 can have a first sheath lumen 101_{L1} and the second sheath 113 can have a second sheath lumen 113_{L}. The method can include creating a space 105 between the target and the engager 106 by expanding the engager 106. The method can include deflecting a distal tip of the second sheath 113 transversely across the first sheath lumen 101_{L1} and into the space 105. The method can include advancing a third sheath 117 through the second sheath lumen 113_{L} and into the target. The third sheath 117 can have a third sheath lumen 117_{L}. The method can include advancing a tool through the third sheath lumen 117_{L} into the target. The tool can be a guidewire 119.

While the devices 100 illustrated in FIGS. 1-34B can be used in the field of ERCP and gastrointestinal anatomy, the devices 100 can be used in any lumenal structure (e.g., lung, ureter), as the main difference in anatomical areas is the diameter size of the endoscope. The endoscope 121 can selected based on the anatomical area that the device 100 is going to be used to access.

To minimize the contamination of the lumens of the device 100 by body fluids or fluids from the endoscope (e.g., endoscope lens flush and/or irrigation fluid), the lumens of the device 100 can be coated with an antimicrobial coating. The antimicrobial coating can be, for example, Microban™ or silver ions.

Any of the features disclosed, contemplated, and/or illustrated herein can be combined in any combination with each other. For example, the features in FIGS. 1-34B can be combined with each other in any combination.

Access devices are disclosed. For example, an ERCP assist device is disclosed. The ERCP assist device can have an outer sheath, an endoscope lumen through which an endoscope can pass, auxiliary lumens, a moveable working channel, a radially expanding (e.g., two radially expanding) stabilizer, an expander (e.g., a balloon) which can expand the stabilizer, or any combination thereof. When the expander (e.g., balloon) expands, the stabilizer can expand by buckling. The moveable working channel can be actuated by a wire or by a balloon. The moveable working channel can be replaced with a balloon which is shaped to guide instruments to the Ampulla of Vater. The endoscope lumen may not be present. A camera can be attached to the outer sheath. The outer sheath can have multiple lumens.

The claims are not limited to the exemplary variations shown in the drawings, but instead may claim any feature disclosed or contemplated in the disclosure as a whole. Any elements described herein as singular can be pluralized (i.e., anything described as “one” can be more than one). Any species element of a genus element can have the characteristics or elements of any other species element of that genus. Some elements may be absent from individual figures for reasons of illustrative clarity. The above-described configurations, elements or complete assemblies and methods and their elements for carrying out the disclosure, and variations of aspects of the disclosure can be combined and modified with each other in any combination, and each combination is hereby explicitly disclosed. All devices, apparatuses, systems, and methods described herein can be used for medical (e.g., diagnostic, therapeutic or rehabilitative) or non-medical purposes. The words “may” and “can” are interchangeable (e.g., “may” can be replaced with “can” and “can” can be replaced with “may”). Any range disclosed can include any subrange of the range disclosed, for example, a range of 1-10 units can include 2-10 units, 8-10 units, or any other subrange. Any phrase involving an “A and/or B” construction can mean (1) A alone, (2) B alone, (3) A and B together, or any combination of (1), (2), and (3), for example, (1) and (2), (1) and (3), (2) and (3), and (1), (2), and (3). The term about can include any tolerance that would be understood by one or ordinary skill in the art, for example, plus or minus 5% of the stated value.

We claim:

1. An access device comprising:
a first sheath having a first sheath lumen;
a second sheath having a second sheath lumen, wherein the second sheath is deflectable into and out of the first sheath lumen, wherein the second sheath has a deflected configuration and a non-deflected configuration, wherein when the second sheath is in the deflected configuration, a second sheath first portion is in the first sheath lumen and extends across the first sheath lumen, and wherein when the second sheath is in the non-deflected configuration, the second sheath first portion is out of the first sheath lumen; and
an engager, wherein the engager is expandable and contractible, wherein the engager has an expanded configuration and a contracted configuration, wherein when the engager is in the expanded configuration, a space is between the engager and the first sheath, wherein when the engager is in the expanded configuration, a second sheath second portion is deflectable into the space, wherein when the engager is in the expanded configuration and when the second sheath is in the deflected configuration, the second sheath second portion is in the space, and wherein when the engager is in the expanded configuration and when the second sheath is in the non-deflected configuration, the second sheath second portion is out of the space.

2. The access device of claim 1, wherein the second sheath second portion comprises a distal terminal end of the second sheath.

3. The access device of claim 1, wherein when the second sheath is in the non-deflected configuration, the second sheath first portion is straight, and wherein when the second sheath is in the deflected configuration, the second sheath first portion is curved.

4. The access device of claim 1, wherein when the second sheath is in the deflected configuration, the second sheath first portion is more curved than when the second sheath is in the non-deflected configuration.

5. The access device of claim 1, wherein when the second sheath is in the deflected configuration, the second sheath first and second portions define a hook shape.

6. The access device of claim 1, wherein when the second sheath is in the non-deflected configuration, the second sheath first portion is parallel to the first sheath lumen.

7. The access device of claim 1, wherein the first sheath further comprises a first sheath second lumen, and wherein the second sheath is in the first sheath second lumen.

8. The access device of claim 1, wherein the engager comprises a stabilizer, wherein the stabilizer has an expanded configuration and a contracted configuration, wherein the stabilizer is moveable from the contracted configuration to the expanded configuration, and wherein when the stabilizer is in the expanded configuration, the stabilizer is farther from the first sheath than when the stabilizer is in the contracted configuration.

9. The access device of claim 1, wherein the engager comprises an expander and a stabilizer, wherein the expander is expandable and contractible, wherein the expander has an expanded configuration and a contracted configuration, wherein when the expander is in the expanded configuration, the engager is in the expanded configuration, wherein when the expander is in the contracted configuration, the engager is in the contracted configuration, wherein the stabilizer has an expanded configuration and a contracted configuration, wherein the stabilizer is moveable from the contracted configuration to the expanded configuration via the expander, wherein when the stabilizer is in the expanded configuration, the stabilizer is farther from the first sheath than when the stabilizer is in the contracted configuration.

10. The access device of claim 9, wherein the expander comprises a balloon.

11. The access device of claim 1, further comprising an endoscope, a third sheath, and a guidewire, wherein the endoscope is moveable in the first sheath lumen, and wherein the third sheath and the guidewire are moveable in the second sheath lumen when the second sheath is in the deflected configuration.

12. The access device of claim 1, further comprising a third sheath having a third sheath first lumen and a third sheath second lumen, wherein the first sheath is in the third sheath first lumen, and wherein the second sheath is in the third sheath second lumen.

13. The access device of claim 12, wherein the first sheath is a torque carrier.

14. The access device of claim 1, further comprising a torque carrier attached to the first sheath.

15. An access device comprising:
a first sheath having a first sheath lumen and a first sheath distal tip, wherein the first sheath distal tip is moveable away from and toward the first sheath lumen, wherein the first sheath has a first sheath deflected configuration and a first sheath non-deflected configuration, wherein when the first sheath is in the first sheath deflected configuration, the first sheath distal tip is farther from the first sheath lumen than when the first sheath is in the first sheath non-deflected configuration;
a second sheath having a second sheath lumen, wherein the second sheath is deflectable into and out of the first sheath lumen, wherein the second sheath has a second sheath deflected configuration and a second sheath non-deflected configuration, wherein when the second sheath is in the second sheath deflected configuration, a second sheath first portion is inside the first sheath lumen and extends across the first sheath lumen, and wherein when the second sheath is in the second sheath non-deflected configuration, the second sheath first portion is outside the first sheath lumen; and
a stabilizer, wherein the stabilizer is expandable and contractible, wherein the stabilizer has an expanded configuration and a contracted configuration, wherein the stabilizer is moveable from the contracted configuration to the expanded configuration via the first sheath distal tip, wherein when the first sheath is in the first sheath non-deflected configuration, the stabilizer is in the contracted configuration, wherein when the first sheath is in the first sheath deflected configuration, the stabilizer is in the expanded configuration, and wherein when the stabilizer is in the expanded configuration, the stabilizer is farther from a first sheath lumen longitudinal axis than when the stabilizer is in the contracted configuration.

16. The access device of claim 15, wherein the stabilizer comprises a stabilizer first end and a stabilizer second end, wherein when the stabilizer is in the expanded configuration, the stabilizer first end is closer to the first sheath lumen longitudinal axis than the stabilizer second end.

17. The access device of claim 15, further comprising an endoscope, a third sheath, and a guidewire, wherein the endoscope is moveable in the first sheath lumen, and wherein the third sheath and the guidewire are moveable in second sheath lumen when the second sheath is in the deflected configuration.

18. The access device of claim 15, wherein when the stabilizer is in the expanded configuration, a space is between the stabilizer and the first sheath, wherein when the stabilizer is in the expanded configuration, a second sheath second portion is deflectable into the space, wherein when the stabilizer is in the expanded configuration and when the second sheath is in the deflected configuration, the second sheath second portion is in the space, and wherein when the stabilizer is in the expanded configuration and when the second sheath is in the non-deflected configuration, the second sheath second portion is out of the space.

19. A method of accessing a target in a body lumen comprising:
advancing a first sheath, a second sheath, and an engager to the target, wherein the first sheath has a first sheath lumen and the second sheath has a second sheath lumen;
creating a space between the target and the engager by expanding the engager;
deflecting a distal tip of the second sheath transversely across the first sheath lumen and into the space;
advancing a third sheath through the second sheath lumen and into the target, wherein the third sheath has a third sheath lumen; and
advancing a tool through the third sheath lumen into the target.

20. The method of claim 19, wherein the tool is a guidewire.
